(19) United States
(12) Patent Application Publication
(10) Pub. No.: US 2022/0275384 A1
(43) Pub. Date: Sep. 1, 2022

(54) ABIOTIC STRESS TOLERANT PLANTS AND METHODS

(71) Applicants: PIONEER OVERSEAS CORPORATION, JOHNSTON, IA (US); SINOBIOWAY BIO-AGRICULTURE GROUP CO. LTD., BEIJING (CN)

(72) Inventors: GUIHUA LU, SAN DIEGO, CA (US); GUANGWU CHEN, BEIJING (CN); ZANTANG LI, BEIJING (CN); JUNHUA LIU, BEIJING (CN); GUANFAN MAO, BEIJING (CN); YANLONG SHI, BEIJING (CN); CHANGGUI WANG, BEIJING (CN); GUOKUI WANG, BEIJING (CN); YU ZHANG, BEIJING (CN)

(73) Assignees: PIONEER OVERSEAS CORPORATION, JOHNSTON, IA (US); SINOBIOWAY BIO-AGRICULTURE GROUP CO. LTD., BEIJING (CN)

(21) Appl. No.: 17/634,132

(22) PCT Filed: Sep. 2, 2019
(86) PCT No.: PCT/CN2019/103934

§ 371 Date: Feb. 9, 2022

(51) Int. Cl. C12N 15/82; C07K 14/415; C12N 9/22

(52) U.S. Cl. CPC C12N 15/8213; C07K 14/415; C12N 15/8218; C12N 15/8273; C12N 9/22

(57) ABSTRACT

Provided are suppression DNA constructs and CRISPR/Cas9 DNA constructs are useful for conferring improved drought tolerance, yield. Compositions (such as plants or seeds) comprising these constructs; and methods utilize these constructs.

### FIELD

The field of the disclosure relates to plant breeding and genetics and, particularly, relates to improving tolerance to abiotic stress in plants.

### BACKGROUND

Stresses to plants may be caused by both biotic and abiotic agents. For example, biotic causes of stress include infection with pathogen, insect feeding, and parasitism by another plant such as mistletoe. Abiotic stresses include, for example, excessive or insufficient available water, temperature extremes, and synthetic chemicals such as herbicides.

Abiotic stress is the primary cause of crop loss worldwide, causing average yield losses more than 50% for major crops (Boyer, J. S. (1982) Science 218:443-448; Bray, E. A. et al. (2000) In Biochemistry and Molecular Biology of Plants, edited by Buchannan, B. B. et al., Amer. Soc. Plant Biol., pp. 1158-1249).

Accordingly, there is a need to develop compositions and methods that increase tolerance to abiotic stress in plants. This invention provides such compositions and methods.

### SUMMARY

The following embodiments are among those encompassed by the disclosure:

In one embodiment, the present disclosure provides a suppression DNA construct comprising at least one heterologous regulatory element operably linked to suppression elements, wherein the suppression elements decrease the expression of an endogenous target polynucleotide encoding a polypeptide comprising an amino acid sequence of at least 90% sequence identity to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152. In certain embodiments, the suppression elements comprise at least 100 contiguous base pairs of a polynucleotide encoding a polypeptide comprising an amino acid sequence of at least 90% sequence identity to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152. In certain embodiments, the suppression elements comprise the polynucleotide sequence of SEQ ID NO: 1, 2, 4, 5, 7, 8, 10, 11, 13, 14, 16, 17, 19, 20, 22, 23, 25, 26, 28, 29, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151.

The present disclosure also provides a CRISPR/Cas construct comprising at least one heterologous regulatory sequence operably linked to gRNA, wherein the gRNA is targeted to a genomic region containing an DN-DRT20, EIN3-1, CYP-1, NAC67-3, DN-DTP21, SIP1, DC1D1, TNS1, SAUR27, or HIP1 gene and/or its regulatory elements to reduce the expression or activity of an endogenous DN-DRT20, EIN3-1, CYP-1, NAC67-3, DN-DTP21, SIP1, DC1D1, TNS1, SAUR27, or HIP1 polypeptide. In certain embodiments, the endogenous gene encodes a polypeptide with amino acid sequence of at least 90% identity to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152. In certain embodiments, the DN-DRT20, EIN3-1, CYP-1, NAC67-3, DN-DTP21, SIP1, DC1D1, TNS1, SAUR27, or HIP1 gene comprises a polynucleotide with nucleotide sequence of SEQ ID NO: 1, 2, 4, 5, 7, 8, 10, 11, 13, 14, 16, 17, 19, 20, 22, 23, 25, 26, 28, 29, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151 or an allelic variant thereof comprising 1 to about 10 nucleotide changes.

The present disclosure further provides a modified plant or seed having decreased expression or activity of an endogenous DN-DRT20, EIN3-1, CYP-1, NAC67-3, DN-DTP21, SIP1, DC1D1, TNS1, SAUR27, or HIP1 polypeptide. In certain embodiments, the modified plant or seed comprises a suppression DNA construct comprising at least one heterologous regulatory element operably linked to suppression elements, wherein the suppression elements decrease the expression of the endogenous DN-DRT20, EIN3-1, CYP-1, NAC67-3, DN-DTP21, SIP1, DC1D1, TNS1, SAUR27, or HIP1 polypeptide. In certain embodiments, the polypeptide comprises an amino acid sequence of at least 90% sequence identity to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152. In certain embodiments, the suppression elements comprise at least 100 contiguous base pairs of a polynucleotide encoding an amino acid sequence of at least 90% sequence identity to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152. In certain embodiments, the suppression elements comprise the polynucleotide of SEQ ID NO: 1, 2, 4, 5, 7, 8, 10, 11, 13, 14, 16, 17, 19, 20, 22, 23, 25, 26, 28, 29, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151.

In certain embodiments, the modified plant or seed comprises a targeted genetic modification at a genomic locus comprising a polynucleotide encoding a DN-DRT20, EIN3-1, CYP-1, NAC67-3, DN-DTP21, SIP1, DC1D1, TNS1, SAUR27, or HIP1 polypeptide, wherein the genetic modification decreases the expression and/or activity of the polypeptide. In certain embodiments, the polynucleotide encodes a polypeptide comprising an amino acid sequence of at least 90% sequence identity to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152.

In certain embodiments, the modified plant or seed exhibits at least one phenotype selected from the group consisting of: increased drought tolerance, increased grain yield, or increased abiotic stress tolerance. In certain embodiments, the modified plant or seed having decreased expression and/or activity of a DN-DRT20, EIN3-1, CYP-1, NAC67-3, DN-DTP21, SIP1, DC1D1, TNS1, SAUR27, or HIP1 polypeptide has increased drought tolerance, increased grain yield, and/or increased abiotic stress tolerance.

In certain embodiments, the plant of the compositions and methods described herein is selected from the group consisting of rice, maize, soybean, sunflower, sorghum, canola, wheat, alfalfa, cotton, barley, millet, sugar cane and switchgrass.

Also provided are methods for increasing drought tolerance in a plant, the method comprising decreasing the expression and/or activity of at least one polynucleotide encoding a DN-DRT20, EIN3-1, CYP-1, NAC67-3, DN-DTP21, SIP1, DC1D1, TNS1, SAUR27, or HIP1 polypeptide in the plant. In certain embodiments, the polypeptide comprises an amino acid sequence of at least 80% sequence identity to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152.

In certain embodiments, the method for increasing drought tolerance comprises: (a) introducing into a regenerable plant cell a suppression DNA construct, wherein the suppression DNA construct comprises at least one heterologous regulatory element operably linked to suppression elements; (b) regenerating a modified plant from the regenerable plant cell, wherein the plant comprises the suppression DNA construct. In certain embodiments, the suppression elements decrease the expression of an endogenous target polynucleotide with amino acid sequence of at least 90% sequence identity to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152. In certain embodiments, the suppression elements comprise at least 100 contiguous base pairs of a polynucleotide with amino acid sequence of at least 90% sequence identity to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152. In certain embodiments, the suppression elements comprise a polynucleotide of SEQ ID NO: 1, 2, 4, 5, 7, 8, 10, 11, 13, 14, 16, 17, 19, 20, 22, 23, 25, 26, 28, 29, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151.

In certain embodiments, the method for increasing drought tolerance comprises: (a) introducing into a regenerable plant cell a targeted genetic modification at a genomic locus comprising a polynucleotide encoding a DN-DRT20, EIN3-1, CYP-1, NAC67-3, DN-DTP21, SIP1, DC1D1, TNS1, SAUR27, or HIP1 polypeptide; and (b) generating the plant, wherein the plant comprises in its genome the introduced genetic modification and has decreased expression and/or activity of the polypeptide. In certain embodiments, the polypeptide comprises an amino acid sequence of at least 80% sequence identity, when compared to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152. In certain embodiments, the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), an engineered site-specific meganucleases, or an Argonaute. In certain embodiments, the targeted genetic modification is present in (a) the coding region; (b) a non-coding region; (c) a regulatory sequence; (d) an untranslated region; or (e) any combination of (a)-(d) of the genomic locus that encodes a polypeptide comprising an amino acid sequence that is at 80% sequence identity, when compared to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152.

In certain embodiments, the targeted genetic modification is introduced by a CRISPR/Cas construct comprising at least one heterologous regulatory sequence operably linked to gRNA, wherein the gRNA is targeted to the endogenous DN-DRT20, EIN3-1, CYP-1, NAC67-3, DN-DTP21, SIP1, DC1D1, TNS1, SAUR27, or HIP1 gene and/or its regulatory elements.

### BRIEF DESCRIPTION OF THE SEQUENCE LISTING

The disclosure can be more fully understood from the following detailed description and Sequence Listing which form a part of this application. The sequence descriptions and sequence listing attached hereto comply with the rules governing nucleotide and amino acid sequence disclosures in patent applications as set forth in 37 C.F.R. §§ 1.821 and 1.825. The sequence descriptions comprise the three letter codes for amino acids as defined in 37 C.F.R. §§ 1.821 and 1.825, which are incorporated herein by reference.

TABLE 1
Sequence Listing Description

| Source/ | Clone | SEQ ID NO: | SEQ ID NO: |
|---|---|---|---|
| Plant species | Designation | (Nucleotide) | (Amino Acid) |
| Oryza sativa | OsDN-DRT20 | 1, 2 | 3 |
| Oryza sativa | OsEIN3-1 | 4, 5 | 6 |
| Oryza sativa | OsCYP-1 | 7, 8 | 9 |
| Oryza sativa | OsNAC67-3 | 10, 11 | 12 |
| Oryza sativa | OsDN-DTP21 | 13, 14 | 15 |
| Oryza sativa | OsSIP1 | 16, 17 | 18 |
| Oryza sativa | OsDC1D1 | 19, 20 | 21 |
| Oryza sativa | OsTNS1 | 22, 23 | 24 |
| Oryza sativa | OsSAUR27 | 25, 26 | 27 |
| Oryza sativa | OsHIP1 | 28, 29 | 30 |
| Artificial | Gene clone primers | 31-50 | n/a |
| Artificial | RT-PCR primers | 51-60 | n/a |
| Oryza sativa | OsDN-DRT20 Paralog | 61 | 62 |
| Zea mays | OsDN-DRT20 Homolog | 63 | 64 |
| Sorghum bicolor | OsDN-DRT20 Homolog | 65 | 66 |
| Glycine max | OsDN-DRT20 Homolog | 67 | 68 |
| Oryza sativa | OsEIN3-1 Paralog | 69 | 70 |
| Zea mays | OsEIN3-1 Homolog | 71 | 72 |
| Sorghum bicolor | OsEIN3-1 Homolog | 73 | 74 |
| Arabidopsis thaliana | OsEIN3-1 Homolog | 75 | 76 |
| Glycine max | OsEIN3-1 Homolog | 77 | 78 |
| Oryza sativa | OsCYP-1 Paralog | 79 | 80 |
| Zea mays | OsCYP-1 Homolog | 81 | 82 |
| Sorghum bicolor | OsCYP-1 Homolog | 83 | 84 |
| Arabidopsis thaliana | OsCYP-1 Homolog | 85 | 86 |
| Glycine max | OsCYP-1 Homolog | 87 | 88 |
| Oryza sativa | OsNAC67-3 Paralog | 89 | 90 |
| Zea mays | OsNAC67-3 Homolog | 91 | 92 |
| Sorghum bicolor | OsNAC67-3 Homolog | 93 | 94 |
| Arabidopsis thaliana | OsNAC67-3 Homolog | 95 | 96 |
| Glycine max | OsNAC67-3 Homolog | 97 | 98 |
| Oryza sativa | OsDN-DTP21 Paralog | 99 | 100 |
| Zea mays | OsDN-DTP21 Homolog | 101 | 102 |
| Sorghum bicolor | OsDN-DTP21 Homolog | 103 | 104 |
| Arabidopsis thaliana | OsDN-DTP21 Homolog | 105 | 106 |
| Glycine max | OsDN-DTP21 Homolog | 107 | 108 |
| Oryza sativa | OsSIP1 Paralog | 109 | 110 |
| Zea mays | OsSIP1 Homolog | 111 | 112 |
| Sorghum bicolor | OsSIP1 Homolog | 113 | 114 |
| Arabidopsis thaliana | OsSIP1 Homolog | 115 | 116 |
| Glycine max | OsSIP1 Homolog | 117 | 118 |
| Oryza sativa | OsDC1D1 Paralog | 119 | 120 |
| Sorghum bicolor | OsDC1D1 Homolog | 121 | 122 |
| Oryza sativa | OsTNS1 Paralog | 123 | 124 |
| Zea mays | OsTNS1 Homolog | 125 | 126 |
| Sorghum bicolor | OsTNS1 Homolog | 127 | 128 |
| Arabidopsis thaliana | OsTNS1 Homolog | 129 | 130 |
| Glycine max | OsTNS1 Homolog | 131 | 132 |
| Oryza sativa | OsSAUR27 Paralog | 133 | 134 |
| Zea mays | OsSAUR27 Homolog | 135 | 136 |
| Sorghum bicolor | OsSAUR27 Homolog | 137 | 138 |
| Arabidopsis thaliana | OsSAUR27 Homolog | 139 | 140 |
| Glycine max | OsSAUR27 Homolog | 141 | 142 |
| Oryza sativa | OsHIP1 Paralog | 143 | 144 |
| Zea mays | OsHIP1 Homolog | 145 | 146 |
| Sorghum bicolor | OsHIP1 Homolog | 147 | 148 |
| Arabidopsis thaliana | OsHIP1 Homolog | 149 | 150 |
| Glycine max | OsHIP1 Homolog | 151 | 152 |

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants; reference to “a cell” includes one or more cells and equivalents thereof known to those skilled in the art, and so forth.

### Definitions

As used herein, “increased drought tolerance” of a plant refers to any measurable improvement in a physiological or physical characteristic, such as yield, as measured relative to a reference or control plant when grown under drought conditions. Typically, when a plant comprising a suppression DNA construct or DNA modification in its genome exhibits increased drought tolerance relative to a reference or control plant, the reference or control plant does not comprise in its genome the suppression DNA construct or DNA modification.

“Agronomic characteristic” is a measurable parameter including but not limited to: greenness, grain yield, growth rate, total biomass or rate of accumulation, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, tiller number, panicle size, early seedling vigor and seedling emergence under low temperature stress.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

A “control,” “control plant,” or “control plant cell” or the like provides a reference point for measuring changes in phenotype of a subject plant or plant cell in which genetic alteration, such as transformation, has been affected as to a gene of interest. For example, a control plant may be a plant having the same genetic background as the subject plant except for the genetic alteration that resulted in the subject plant or cell.

“Plant” includes reference to whole plants, plant organs, plant tissues, seeds and plant cells and progeny of the same. Plant cells include, without limitation, cells from seeds, suspension cultures, embryos, meristematic regions, callus tissues, leaves, roots, shoots, gametophytes, sporophytes, pollen, and microspores.

“Progeny” comprises any subsequent generation of a plant.

“Modified plant” includes reference to a plant which comprises within its genome a heterologous polynucleotide or modified gene or promoter. For example, the heterologous polynucleotide is stably integrated within the genome such that the polynucleotide is passed on to successive generations. The heterologous polynucleotide may be integrated into the genome alone or as part of a recombinant DNA construct.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, and “nucleic acid fragment” are used interchangeably and refer to a polymer of RNA or DNA that is single- or double-stranded, optionally containing synthetic, non-natural or altered nucleotide bases. Nucleotides (usually found in their 5-monophosphate form) are referred to by their single-letter designation as follows: “A” for adenylate or deoxyadenylate, “C” for cytidylate or deoxycytidylate, and “G” for guanylate or deoxyguanylate for RNA or DNA, respectively; “U” for uridylate; “T” for deoxythymidylate; “R” for purines (A or G); “Y” for pyrimidines (C or T); “K” for G or T; “H” for A or C or T; “I” for inosine; and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, and sulfation, gamma-carboxylation of glutamic acid residues, hydroxylation and ADP-ribosylation.

“Recombinant DNA construct” refers to a combination of nucleic acid fragments that are not normally found together in nature. Accordingly, a recombinant DNA construct may comprise regulatory elements and coding sequences that are derived from different sources, or regulatory elements and coding sequences derived from the same source, but arranged in a manner different than that normally found in nature.

“Regulatory elements” refer to nucleotide sequences located upstream (5′ non-coding sequences), within, or downstream (3′ non-coding sequences) of a coding sequence, and influencing the transcription, RNA processing or stability, or translation of the associated coding sequence. Regulatory elements may include, but are not limited to, promoters, translation leader sequences, introns, and poly-adenylation recognition sequences. The terms “regulatory sequence” and “regulatory element” and “regulatory region” are used interchangeably herein.

“Promoter” refers to a nucleic acid fragment capable of controlling transcription of another nucleic acid fragment. “Promoter functional in a plant” is a promoter capable of controlling transcription of genes in plant cells whether or not its origin is from a plant cell. “Tissue-specific promoter” and “tissue-preferred promoter” refers to a promoter that is expressed predominantly but not necessarily exclusively in one tissue or organ, but that may also be expressed in one specific cell or cell type. “Developmentally regulated promoter” is a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

As used herein “increased”, “increase”, or the like refers to any detectable increase in an experimental group (e.g., plant with a DNA modification described herein) as compared to a control group (e.g., wild-type plant that does not comprise the DNA modification). Accordingly, increased expression of a protein comprises any detectable increase in the total level of the protein in a sample and can be determined using routine methods in the art such as, for example, Western blotting and ELISA.

As used herein, “yield” refers to the amount of agricultural production harvested per unit of land, and may include reference to bushels per acre or kilograms per mu of a crop at harvest, as adjusted for grain moisture (e.g., typically 15% for maize, 13.5% for rice). Grain moisture is measured in the grain at harvest. The adjusted test weight of grain is determined to be the weight in pounds per bushel or grams per plant, adjusted for grain moisture level at harvest.

A “suppression DNA construct” is a recombinant DNA construct which when transformed or stably integrated into the genome of the plant, results in “silencing” of a target gene in the plant. The target gene may be endogenous or transgenic to the plant.

“Silencing”, as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, includes lowering, reducing, declining, decreasing, inhibiting, eliminating or preventing.

Suppression DNA constructs are well-known in the art, and may be readily constructed once the target gene of interest is selected, and include, without limitation, co-suppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as siRNA (short interfering RNA) constructs and miRNA (microRNA) constructs.

“Antisense inhibition” refers to the production of antisense RNA transcripts capable of suppressing the expression of the target gene or gene product. “Co-suppression” refers to the production of sense RNA transcripts capable of suppressing the expression of the target gene or gene product. “Sense” RNA refers to RNA transcript that includes the mRNA and can be translated into protein within a cell or in vitro. Another variation describes the use of plant viral sequences to direct the suppression of proximal mRNA encoding sequences (PCT Publication No. WO 98/36083 published on Aug. 20, 1998).

RNA interference (RNAi) refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

As used herein, “sequence identity” or “identity” in the context of two polynucleotides or polypeptide sequences make reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” is calculated by determining the number of positions at which the identical nucleic acid base or amino acid residue occurs in both sequences to yield the number of matched positions, dividing the number of matched positions by the total number of positions in the window of comparison, and multiplying the result by 100.

Unless stated otherwise, multiple alignments of the sequences provided herein are performed using the Clustal V method of alignment (Higgins and Sharp. (1989) CAB/OS. 5:151-153) with the default parameters (GAP PENALTY=10, GAP LENGTH PENALTY=10). Default parameters for pairwise alignments and calculation of percent identity of amino acid sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

### Compositions:

The present disclosure provides constructs to decrease the expression and/or activity of a DN-DRT20, EIN3-1, CYP-1, NAC67-3, DN-DTP21, SIP1, DC1D1, TNS1, SAUR27, or HIP1 polypeptide.

In one aspect of the disclosure, the polypeptide comprises an amino acid sequence that is at least 80% identical (e.g. 81%, 82%, 83%, 84%, 85%, 86%, 87%, 88%, 89%, 90%, 91%, 92%, 93%, 94%, 95%, 96%, 97%, 98%, 99%, or 100%) to an amino acid sequence of any one of SEQ ID NO: 3 (OsDN-DRT20), SEQ ID NO: 6 (OsEIN3-1), SEQ ID NO: 9 (OsCYP-1), SEQ ID NO: 12 (OsNAC67-3), SEQ ID NO: 15 (OsDN-DTP21), SEQ ID NO: 18 (OsSIP1), SEQ ID NO: 21 (OsDC1D1), SEQ ID NO: 24 (OsTNS1), SEQ ID NO: 27 (OsSAUR27), and SEQ ID NO: 30 (OsHIP1).

“OsDN-DRT20” refers to a rice polypeptide that confers drought sensitive phenotype when overexpressed. The OsDN-DRT20 polypeptide (SEQ ID NO: 3) is encoded by the coding sequence (CDS) (SEQ ID NO: 2) or nucleotide sequence (SEQ ID NO: 1) at rice gene locus LOC_Os02g51760.1, which is annotated as “expressed protein” in TIGR. “DN-DRT20 polypeptide” refers herein to the OsDN-DRT20 polypeptide and its paralogs (e.g., SEQ ID NO: 62 encoded by SEQ ID NO: 61) or homologs from other organisms, such as maize (SEQ ID NO: 64 encoded by SEQ ID NO: 63), sorghum (SEQ ID NO: 66 encoded by SEQ ID NO: 65), or soybean (SEQ ID NO: 68 encoded by SEQ ID NO: 67).

“OsEIN3-1” refers to a rice polypeptide that confers drought sensitive phenotype when overexpressed. The OsEIN3-1 polypeptide (SEQ ID NO: 6) is encoded by the coding sequence (CDS) (SEQ ID NO: 5) or nucleotide sequence (SEQ ID NO: 4) at rice gene locus LOC_Os03g20790, which is annotated as “ethylene-insensitive 3, putative, expressed” in TIGR. “EIN3-1 polypeptide” refers herein to the OsEIN3-1 polypeptide and its paralogs (e.g., SEQ ID NO: 70 encoded by SEQ ID NO: 69) or homologs from other organisms, such as maize (SEQ ID NO: 72 encoded by SEQ ID NO: 71), sorghum (SEQ ID NO: 74 encoded by SEQ ID NO: 73), Arabidopsis (SEQ ID NO: 76 encoded by SEQ ID NO: 75), or soybean (SEQ ID NO: 78 encoded by SEQ ID NO: 77).

“OsCYP-1” refers to a rice polypeptide that confers drought sensitive phenotype when overexpressed. The OsCYP-1 polypeptide (SEQ ID NO: 9) is encoded by the coding sequence (CDS) (SEQ ID NO: 8) or nucleotide sequence (SEQ ID NO: 7) at rice gene locus LOC_Os02g47470.1, which is annotated as “cytochrome P450, putative, expressed” in TIGR. “CYP-1 polypeptide” refers herein to the OsCYP-1 polypeptide and its paralogs (e.g., SEQ ID NO: 80 encoded by SEQ ID NO: 79) or homologs from other organisms, maize (SEQ ID NO: 82 encoded by SEQ ID NO: 81), sorghum (SEQ ID NO: 84 encoded by SEQ ID NO: 83), Arabidopsis (SEQ ID NO: 86 encoded by SEQ ID NO: 85), or soybean (SEQ ID NO: 88 encoded by SEQ ID NO: 87).

“OsNAC67-3” refers to a rice polypeptide that confers drought sensitive phenotype when overexpressed. The OsNAC67-3 polypeptide (SEQ ID NO: 12) is encoded by the coding sequence (CDS) (SEQ ID NO: 11) or nucleotide sequence (SEQ ID NO: 10) at rice gene locus LOC_Os01g66120.1, which is annotated as “No apical meristem protein, putative, expressed” in TIGR. “NAC67-3polypeptide” refers herein to the OsNAC67-3 polypeptide and its paralogs (e.g., SEQ ID NO: 90 encoded by SEQ ID NO: 89) or homologs from other organisms, such as maize (SEQ ID NO: 92 encoded by SEQ ID NO: 91), sorghum (SEQ ID NO: 94 encoded by SEQ ID NO: 93), Arabidopsis (SEQ ID NO: 96 encoded by SEQ ID NO: 95), or soybean (SEQ ID NO: 98 encoded by SEQ ID NO: 97).

“OsDN-DTP21” refers to a rice polypeptide that confers drought sensitive phenotype when overexpressed. The OsDN-DTP21 polypeptide (SEQ ID NO: 15) is encoded by the coding sequence (CDS) (SEQ ID NO: 14) or nucleotide sequence (SEQ ID NO: 13) at rice gene locus LOC_Os09g39370.1, which is annotated as “expressed protein” in TIGR. “DN-DTP21 polypeptide” refers herein to the OsDN-DTP21 polypeptide and its paralogs (e.g., SEQ ID NO: 100 encoded by SEQ ID NO: 99) or homologs from other organisms, such as maize (SEQ ID NO: 102 encoded by SEQ ID NO: 101), sorghum (SEQ ID NO: 104 encoded by SEQ ID NO: 103), Arabidopsis (SEQ ID NO: 106 encoded by SEQ ID NO: 105), or soybean (SEQ ID NO: 108 encoded by SEQ ID NO: 107).

“OsSIP1” refers to a rice polypeptide that confers drought sensitive phenotype when overexpressed. The OsSIP1 polypeptide (SEQ ID NO: 18) is encoded by the coding sequence (CDS) (SEQ ID NO: 17) or nucleotide sequence (SEQ ID NO: 16) at rice gene locus LOC_Os07g04150.1, which is annotated as “stress-induced protein, putative, expressed” in TIGR. “SIP1 polypeptide” refers herein to the OsSIP1 polypeptide and its paralogs (e.g., SEQ ID NO: 110 encoded by SEQ ID NO: 109) or homologs from other organisms, such as maize (SEQ ID NO: 112 encoded by SEQ ID NO: 111), sorghum (SEQ ID NO: 114 encoded by SEQ ID NO: 113), Arabidopsis (SEQ ID NO: 116 encoded by SEQ ID NO: 115), or soybean (SEQ ID NO: 118 encoded by SEQ ID NO: 117).

“OsDC1 D1” refers to a rice polypeptide that confers drought sensitive phenotype when overexpressed. The OsDC1 D1 polypeptide (SEQ ID NO: 21) is encoded by the coding sequence (CDS) (SEQ ID NO: 20) or nucleotide sequence (SEQ ID NO: 19) at rice gene locus LOC_Os08g15710.1, which is annotated as “DC1 domain, putative, expressed” in TIGR. “DC1 D1 polypeptide” refers herein to the OsDC1 D1 polypeptide and its paralogs (e.g., SEQ ID NO: 120 encoded by SEQ ID NO: 119) or homologs from other organisms, such as sorghum (SEQ ID NO: 122 encoded by SEQ ID NO: 121).

“OsTNS1” refers to a rice polypeptide that confers drought sensitive phenotype when overexpressed. The OsTNS1 polypeptide (SEQ ID NO: 24) is encoded by the coding sequence (CDS) (SEQ ID NO: 23) or nucleotide sequence (SEQ ID NO: 22) at rice gene locus LOC_Os01g49890.1, which is annotated as “threonine synthase, chloroplast precursor, putative, expressed” in TIGR. “TNS1 polypeptide” refers herein to the OsTNS1 polypeptide and its paralogs (e.g., SEQ ID NO: 124 encoded by SEQ ID NO: 123) or homologs from other organisms, such as maize (SEQ ID NO: 126 encoded by SEQ ID NO: 125), sorghum (SEQ ID NO: 128 encoded by SEQ ID NO: 127), Arabidopsis (SEQ ID NO: 130 encoded by SEQ ID NO: 129), or soybean (SEQ ID NO: 132 encoded by SEQ ID NO: 131).

“OsSAUR27” refers to a rice polypeptide that confers drought sensitive phenotype when overexpressed. The OsSAUR27 polypeptide (SEQ ID NO: 27) is encoded by the coding sequence (CDS) (SEQ ID NO: 26) or nucleotide sequence (SEQ ID NO: 25) at rice gene locus LOC_Os06g48850.1, which is annotated as “OsSAUR27—Auxin-responsive SAUR gene family member, expressed” in TIGR. “SAUR27 polypeptide” refers herein to the OsSAUR27 polypeptide and its paralogs (e.g., SEQ ID NO: 134 encoded by SEQ ID NO: 133) or homologs from other organisms, such as maize (SEQ ID NO: 136 encoded by SEQ ID NO: 135), sorghum (SEQ ID NO: 138 encoded by SEQ ID NO: 137), Arabidopsis (SEQ ID NO: 140 encoded by SEQ ID NO: 139), or soybean (SEQ ID NO: 142 encoded by SEQ ID NO: 141).

“OsHIP1” refers to a rice polypeptide that confers drought sensitive phenotype when overexpressed. The OsHIP1 polypeptide (SEQ ID NO: 30) is encoded by the coding sequence (CDS) (SEQ ID NO: 29) or nucleotide sequence (SEQ ID NO: 28) at rice gene locus LOC_Os01g39290.1, which is annotated as “harpin-induced protein 1 domain containing protein, expressed” in TIGR. “HIP1 polypeptide” refers herein to the OsHIP1 polypeptide and its paralogs (e.g., SEQ ID NO: 144 encoded by SEQ ID NO: 143) or homologs from other organisms, such as maize (SEQ ID NO: 146 encoded by SEQ ID NO: 145), sorghum (SEQ ID NO: 148 encoded by SEQ ID NO: 147), Arabidopsis (SEQ ID NO: 150 encoded by SEQ ID NO: 149), or soybean (SEQ ID NO: 152 encoded by SEQ ID NO: 151).

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

### A. Suppression DNA Constructs and CRISPR/Cas Constructs

Provided are suppression DNA constructs that decrease the expression and/or activity of a DN-DRT20, EIN3-1, CYP-1, NAC67-3, DN-DTP21, SIP1, DC1D1, TNS1, SAUR27, or HIP1 polypeptide. In certain embodiments, the suppression DNA construct is a co-suppression construct, antisense construct, viral-suppression construct, hairpin suppression construct, stem-loop suppression construct, double-stranded RNA-producing construct, and more generally, RNAi (RNA interference) construct and small RNA constructs such as siRNA (short interfering RNA) constructs and miRNA (microRNA) constructs.

In certain embodiments, the suppression DNA construct comprises at least one heterologous regulatory element operably linked to suppression elements, wherein the suppression elements suppress the expression of an endogenous target polynucleotide with amino acid sequence of at least 90% sequence identity to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152. In certain embodiments, the suppression elements comprise at least 100 contiguous base pairs of a polynucleotide with amino acid sequence of at least 90% sequence identity to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152. In certain embodiments, the suppression elements comprise the polynucleotide of SEQ ID NO: 1, 2, 4, 5, 7, 8, 10, 11, 13, 14, 16, 17, 19, 20, 22, 23, 25, 26, 28, 29, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151.

The present disclosure also provides a CRISPR/Cas construct comprising at least one heterologous regulatory sequence operably linked to gRNA, wherein the gRNA is targeted to a genomic region containing an endogenous DN-DRT20, EIN3-1, CYP-1, NAC67-3, DN-DTP21, SIP1, DC1D1, TNS1, SAUR27, or HIP1 gene and/or its regulatory elements to reduce the expression or activity of an endogenous DN-DRT20, EIN3-1, CYP-1, NAC67-3, DN-DTP21, SIP1, DC1D1, TNS1, SAUR27, or HIP1 polypeptide. In certain embodiments, the endogenous gene encodes a polypeptide with amino acid sequence of at least 90% identity to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152. Further, the DN-DRT20, EIN3-1, CYP-1, NAC67-3, DN-DTP21, SIP1, DC1D1, TNS1, SAUR27, or HIP1 gene comprises a polynucleotide with nucleotide sequence of SEQ ID NO: 1, 2, 4, 5, 7, 8, 10, 11, 13, 14, 16, 17, 19, 20, 22, 23, 25, 26, 28, 29, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151 or an allelic variant thereof comprising 1 to about 10 nucleotide changes. In certain embodiments, the endogenous regulatory elements comprise a polynucleotide with nucleotide sequence of SEQ ID NO: 1, 2, 4, 5, 7, 8, 10, 11, 13, 14, 16, 17, 19, 20, 22, 23, 25, 26, 28, 29, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151.

In certain embodiments the at least one regulatory element is a heterologous regulatory element. In certain embodiments, the at least one regulatory element of the recombinant DNA construct comprises a promoter. In certain embodiments, the promoter is a heterologous promoter.

A number of promoters can be used in recombinant DNA constructs of the present disclosure. The promoters can be selected based on the desired outcome, and may include constitutive, tissue-specific, inducible, or other promoters for expression in the host organism.

A “constitutive” promoter is a promoter, which is active under most environmental conditions. Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

A tissue-specific or developmentally-regulated promoter is a DNA sequence which regulates the expression of a DNA sequence selectively in the cells/tissues of a plant, such as in those cells/tissues critical to tassel development, seed set, or both, and which usually limits the expression of such a DNA sequence to the developmental period of interest (e.g. tassel development or seed maturation) in the plant. Any identifiable promoter which causes the desired temporal and spatial expression may be used in the methods of the present disclosure.

Many leaf-preferred promoters are known in the art (Yamamoto et al. (1997) Plant J. 12(2):255-265; Kwon et al. (1994) Plant Physiol. 105:357-367; Yamamoto et al. (1994) Plant Cell Physiol. 35(5):773-778; Gotor et al. (1993) Plant J. 3:509-518; Orozco et al. (1993) Plant Mol. Biol. 23(6):1129-1138; and Matsuoka et al. (1993) Proc. Natl. Acad. Sci. USA 90(20):9586-9590).

Promoters which are seed or embryo-specific and may be useful in the disclosure include soybean Kunitz trypsin inhibitor (Kti3, Jofuku and Goldberg. (1989) Plant Cell 1:1079-1093), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564). Promoters of seed-specific genes operably linked to heterologous coding regions in chimeric gene constructions maintain their temporal and spatial expression pattern in transgenic plants. Such examples include Arabidopsis 2S seed storage protein gene promoter to express enkephalin peptides in Arabidopsis and Brassica napus seeds (Vanderkerckhove et al. (1989) Bio/Technology 7: L929-932), bean lectin and bean beta-phaseolin promoters to express luciferase (Riggs et al. (1989) Plant Sci. 63:47-57), and wheat glutenin promoters to express chloramphenicol acetyl transferase (Colot et al. (1987) EMBO J 6:3559-3564).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Also contemplated are synthetic promoters which include a combination of one or more heterologous regulatory elements.

The promoter of the suppression DNA constructs of the invention can be any type or class of promoter known in the art, such that any one of a number of promoters can be used to express the various polynucleotide sequences disclosed herein, including the native promoter of the polynucleotide sequence of interest. The promoters for use in the suppression DNA constructs of the invention can be selected based on the desired outcome.

The suppression DNA constructs of the present disclosure may also include other regulatory elements, including but not limited to, translation leader sequences, introns, and polyadenylation recognition sequences. In certain embodiments, a suppression DNA construct further comprises an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg. (1988) Mol. CellBiol. 8:4395-4405; Callis et al. (1987) Genes Dev. 1:1183-1200).

### B. Plants and Plant Cells

Provided are plants, plant cells, plant parts, seed and grain comprising in its genome any of the suppression DNA constructs described herein, so that the plants, plant cells, plant parts, seed, and/or grain have decreased expression of the encoded polypeptide.

Also provided are plants, plant cells, plant parts, seeds, and grain comprising an introduced genetic modification at a genomic locus that encodes a polypeptide described herein. In certain embodiments, the polypeptide comprises an amino acid sequence that is at least 80% identical to an amino acid sequence selected from the group consisting of SEQ ID NOs: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152. In certain embodiments, the genetic modification decreases the activity of the encoded polypeptide. In certain embodiments, the genetic modification decreases the level of the encoded polypeptide. In certain embodiments, the genetic modification decreases both the level and activity of the encoded polypeptide.

The plant may be a monocotyledonous or dicotyledonous plant, for example, a rice or maize or soybean plant, such as a maize hybrid plant or a maize inbred plant. The plant may also be sunflower, sorghum, canola, wheat, alfalfa, cotton, barley, millet, sugar cane or switchgrass.

In certain embodiments the plant exhibits increased drought tolerance when compared to a control plant. In certain embodiments, the plant exhibits an alteration of at least one agronomic characteristic when compared to the control plant.

One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates.

C. Stacking with Other Traits of Interest

In some embodiments, the inventive polynucleotides disclosed herein are engineered into a molecular stack. Thus, the various host cells, plants, plant cells, plant parts, seeds, and/or grain disclosed herein can further comprise one or more traits of interest. In certain embodiments, the host cell, plant, plant part, plant cell, seed, and/or grain is stacked with any combination of polynucleotide sequences of interest in order to create plants with a desired combination of traits. As used herein, the term “stacked” refers to having multiple traits present in the same plant or organism of interest. For example, “stacked traits” may comprise a molecular stack where the sequences are physically adjacent to each other. A trait, as used herein, refers to the phenotype derived from a particular sequence or groups of sequences. In one embodiment, the molecular stack comprises at least one polynucleotide that confers tolerance to glyphosate. Polynucleotides that confer glyphosate tolerance are known in the art.

In certain embodiments, the molecular stack comprises at least one polynucleotide that confers tolerance to glyphosate and at least one additional polynucleotide that confers tolerance to a second herbicide.

In certain embodiments, the plant, plant cell, seed, and/or grain having an inventive polynucleotide sequence may be stacked with, for example, one or more sequences that confer tolerance to: an ALS inhibitor; an HPPD inhibitor; 2,4-D; other phenoxy auxin herbicides; aryloxyphenoxypropionate herbicides; dicamba; glufosinate herbicides; herbicides which target the protox enzyme (also referred to as “protox inhibitors”).

The plant, plant cell, plant part, seed, and/or grain comprising decreased expression and/or activity of the polypeptides described herein can also be combined with at least one other trait to produce plants that further comprise a variety of desired trait combinations. For instance, the plant, plant cell, plant part, seed, and/or grain may be stacked with polynucleotides encoding polypeptides having pesticidal and/or insecticidal activity, or a plant, plant cell, plant part, seed, and/or grain having an inventive polynucleotide sequence may be combined with a plant disease resistance gene.

These stacked combinations can be created by any method including, but not limited to, breeding plants by any conventional methodology, or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

### Methods:

A. Method for Increasing Drought Tolerance and/or Increasing Grain Yield in a Plant

Provided is a method for increasing drought tolerance and/or increasing grain yield in a plant, comprising decreasing the expression and/or activity of at least one polynucleotide encoding a DN-DRT20, EIN3-1, CYP-1, NAC67-3, DN-DTP21, SIP1, DC1D1, TNS1, SAUR27, or HIP1 polypeptide. In certain embodiments, polynucleotide encodes a polypeptide comprising an amino acid sequence of at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152.

In certain embodiments, the method comprises: (a) expressing in a regenerable plant cell a suppression DNA construct, described herein; and (b) generating the plant, wherein the plant comprises in its genome the suppression DNA construct. In certain embodiments the regulatory element is a heterologous promoter.

In certain embodiments, the method comprises: (a) introducing in a regenerable plant cell a targeted genetic modification at a genomic locus that encodes the polypeptide; and (b) generating the plant, wherein the level and/or activity of the encoded polypeptide is decreased in the plant. In certain embodiments the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), engineered site-specific meganucleases, or Argonaute. In certain embodiments, the targeted genetic modification is present in (a) the coding region; (b) a non-coding region; (c) a regulatory sequence; (d) an untranslated region; or (e) any combination of (a)-(d) of the genomic locus that encodes a polypeptide comprising an amino acid sequence that is at least 80% identical to an amino acid sequence selected from the group consisting of SEQ ID NOs: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152.

The plant for use in the inventive methods can be any plant species described herein. In certain embodiments, the plant is maize, soybean, or rice.

Various methods can be used to introduce a sequence of interest into a plant, plant part, plant cell, seed, and/or grain. “Introducing” is intended to mean presenting to the plant, plant cell, seed, and/or grain the inventive polynucleotide or resulting polypeptide in such a manner that the sequence gains access to the interior of a cell of the plant. The methods of the disclosure do not depend on a particular method for introducing a sequence into a plant, plant cell, seed, and/or grain, only that the polynucleotide or polypeptide gains access to the interior of at least one cell of the plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In other embodiments, the inventive polynucleotides disclosed herein may be introduced into plants by contacting plants with a virus or viral nucleic acids. Generally, such methods involve incorporating a nucleotide construct of the disclosure within a DNA or RNA molecule. It is recognized that the inventive polynucleotide sequence may be initially synthesized as part of a viral polyprotein, which later may be processed by proteolysis in vivo or in vitro to produce the desired recombinant protein. Further, it is recognized that promoters disclosed herein also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide disclosed herein, for example, as part of an expression cassette, stably incorporated into their genome.

Transformed plant cells which are derived by plant transformation techniques, including those discussed above, can be cultured to regenerate a whole plant which possesses the transformed genotype (i.e., an inventive polynucleotide), and thus the desired phenotype, such as increased yield. For transformation and regeneration of maize see, Gordon-Kamm et al., The Plant Cell, 2:603-618 (1990).

Various methods can be used to introduce a genetic modification at a genomic locus that encodes a polypeptide disclosed herein into the plant, plant part, plant cell, seed, and/or grain. In certain embodiments the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In some embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpfl endonuclease systems, and the like. In some embodiments, the introduction of a DSB can be combined with the introduction of a polynucleotide modification template.

A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

In addition to modification by a double strand break technology, modification of one or more bases without such double strand break are achieved using base editing technology, see e.g., Gaudelli et al., (2017) Programmable base editing of A*T to G*C in genomic DNA without DNA cleavage. Nature 551(7681):464-471; Komor et al., (2016) Programmable editing of a target base in genomic DNA without double-stranded DNA cleavage, Nature 533(7603):420-4.

These fusions contain dCas9 or Cas9 nickase and a suitable deaminase, and they can convert e.g., cytosine to uracil without inducing double-strand break of the target DNA. Uracil is then converted to thymine through DNA replication or repair. Improved base editors that have targeting flexibility and specificity are used to edit endogenous locus to create target variations and improve grain yield. Similarly, adenine base editors enable adenine to inosine change, which is then converted to guanine through repair or replication. Thus, targeted base changes i.e., C⋅G to T⋅A conversion and A⋅T to G⋅C conversion at one more location made using appropriate site-specific base editors.

In an embodiment, base editing is a genome editing method that enables direct conversion of one base pair to another at a target genomic locus without requiring double-stranded DNA breaks (DSBs), homology-directed repair (HDR) processes, or external donor DNA templates. In an embodiment, base editors include (i) a catalytically impaired CRISPR-Cas9 mutant that are mutated such that one of their nuclease domains cannot make DSBs; (ii) a single-strand-specific cytidine/adenine deaminase that converts C to U or A to G within an appropriate nucleotide window in the single-stranded DNA bubble created by Cas9; (iii) a uracil glycosylase inhibitor (UGI) that impedes uracil excision and downstream processes that decrease base editing efficiency and product purity; and (iv) nickase activity to cleave the non-edited DNA strand, followed by cellular DNA repair processes to replace the G-containing DNA strand.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

TAL effector nucleases (TALEN) are a class of sequence-specific nucleases that can be used to make double-strand breaks at specific target sequences in the genome of a plant or other organism (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H—N—H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type Ils endonuclease such as Fokl. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18 nucleotide recognition sequence.

Genome editing using DSB-inducing agents, such as Cas9-gRNA complexes, has been described, for example in U.S. Patent Application US 2015-0082478 A1, published on Mar. 19, 2015, WO2015/026886 A1, published on Feb. 26, 2015, WO2016007347, published on Jan. 14, 2016, and WO201625131, published on Feb. 18, 2016, all of which are incorporated by reference herein.

### EXAMPLES

The following are examples of specific embodiments of some aspects of the invention. The examples are offered for illustrative purposes only and are not intended to limit the scope of the invention in any way.

### Example 1

### Cloning and Vector Construction of Drought Sensitive Genes

A binary construct that contains four multimerized enhancers elements derived from the Cauliflower Mosaic Virus 35S (CaMV 35S) promoter was used, and the rice activation tagging population was developed from four japonica (Oryza sativa ssp. Japonica) varieties (Zhonghua 11, Chaoyou 1, Taizhong 65 and Nipponbare), which were transformed by Agrobacteria-mediated transformation method as described by Lin and Zhang ((2005) Plant Cell Rep. 23:540-547). The transgenic lines generated were developed and the transgenic seeds were harvested to form the rice activation tagging population.

Drought sensitive tagging lines (ATLs) were confirmed in repeated field experiments and their T-DNA insertion loci were determined by ligation mediated nested PCR, or plasmid rescue or Southern-by-Sequencing method (Zastrow-Hayes G. M. et al. (2015), The Plant Genome, 8:1-15). The genes near by the left border and right border of the T-DNA were cloned and the functional genes were recapitulated by field screens. Only the recapitulated functional genes are shown herein. Based on LOC IDs of the genes shown in Table 2, primers were designed for cloning the rice drought sensitive genes; OsDN-DRT20 (use SEQ ID NOs: 31 and 32), OsEIN3-1 (use SEQ ID NOs: 33 and 34), OsCYP-1 (use SEQ ID NOs: 35 and 36), OsNAC67-3 (use SEQ ID NOs: 37 and 38), OsDN-DTP21 (use SEQ ID NOs: 39 and 40), OsSIP1 (use SEQ ID NOs: 41 and 42), OsDC1D1 (use SEQ ID NOs: 43 and 44), OsTNS1 (use SEQ ID NOs: 45 and 46), OsSAUR27 (use SEQ ID NOs: 47 and 48), and OsHIP1 (use SEQ ID NOs: 49 and 50).

TABLE 2
Rice gene names, Gene IDs (from TIGR) and Construct IDs

| Gene name | LOC ID | Construct ID |
|---|---|---|
| OsDN-DRT20 | LOC_Os02g51760.1 | DP0623 |
| OsEIN3-1 | LOC_Os03g20790 | DP1804 |
| OsCYP-1 | LOC_Os02g47470.1 | DP1365 |
| OsNAC67-3 | LOC_Os01g66120.1 | DP2253 |
| OsDN-DTP21 | LOC_Os09g39370.1 | DP1139 |
| OsSIP1 | LOC_Os07g04150.1 | DP1448 |
| OsDC1D1 | LOC_Os08g15710.1 | DP0865Y |
| OsTNS1 | LOC_Os01g49890.1 | DP0997Y |
| OsSAUR27 | LOC_Os06g48850.1 | DP0908 |
| OsHIP1 | LOC_Os01g39290.1 | DP0925 |

PCR amplified products were extracted after the agarose gel electrophoresis using a column kit and then ligated with TA cloning vectors. The sequences and orientation in these constructs were confirmed by sequencing. Each gene was cloned into a plant binary construct under CaMV 35S promoter except DP2253 and made the overexpression vectors as indicated in Table 2. DP2253 is an overexpression vector of OsNAC67-3 under the root-preferred promoter KT630.

### Example 2

### Transformation and Gene Expression Analysis of Transgenic Rice Lines

Zhonghua 11 (Oryza sativa L.) were transformed with either a vector prepared in Example 1 or an empty vector (DP0158) by Agrobacteria-mediated transformation as described by Lin and Zhang ((2005) Plant Cell Rep. 23:540-547). Transgenic seedlings (T₀) generated in the transformation laboratory were transplanted in field to get T₁ seeds. The T₁ and subsequent T₂ seeds were screened to confirm transformation and positively identified transgenic seeds were used in the following trait screens.

The gene expression levels in the leaves of the transgenic rice plants were determined by RT-PCR. Primers were designed for RT-PCR analyses for OsDN-DRT20 using SEQ ID Nos: 51 and 52, OsNAC67-3 using SEQ ID Nos: 53 and 54, OsSIP1 using SEQ ID Nos: 55 and 56, OsTNS1 using SEQ ID Nos: 57 and 58, and OsHIP1 using SEQ ID Nos: 59 and 60 in their over-expression transgenic rice. The level of expression in ZH11-TC (tissue cultured ZH11 rice) was set at 1.00, and the expression levels in the transgenic plants were compared to ZH11-TC. Gene expression was normalized based on the EF-1α mRNA levels, and the results from the gene expression analysis are provided in Table 3 below.

TABLE 3
Relative Expression Level Fold Increase in Transgenic Rice Plants

| | | Relative Expression |
|---|---|---|
| Gene name | Construct ID | Level Fold Increase |
| OsDN-DRT20 | DP0623 | from 36.23 to 28785.82 |
| OsNAC67-3 | DP2253 | from 0.64 to 6.61 |
| OsSIP1 | DP1448 | from 51.22 to 213.25 |
| OsTNS1 | DP0997Y | from 0.86 to 200.37 |
| OsHIP1 | DP0925 | from 14.57 to 28.63 |

### Example 3

### Characterization of the Transgenic Rice Plants

The transgenic rice plants from Example 2 and ZH11-TC and DP0158 rice plants were tested for: (a) drought tolerance, and (b) grain yield under well-watered conditions.

T₂ seeds from the plants of Example 2 were sterilized by 800 ppm carbendazol for 8 hours at 32° C. and washed 3-5 time, soaked in water for 16 hours at 32° C., and germinated for 18 hours at 35-37° C. in an incubator. Germinated seeds were used as follows for each test:

(a) drought tolerance—germinated seeds were planted in a seedbed field. At 3-leaf stage, the seedlings were transplanted into the testing field with 4 replicates and 10 plants per replicate for each transgenic line, and the 4 replicates were planted in the same block. ZH11-TC and DP0158 seedlings were nearby the transgenic lines in the same block, and were used as controls in the statistical analysis. The rice plants were managed by normal practice using pesticides and fertilizers. Watering was stopped at the panicle initiation stage, so as to give drought stress at flowering stage depending on the weather conditions (temperature and humidity). The soil water content was measured every 4 days at about 10 sites per block using TDR30 (Spectrum Technologies, Inc.). Plant phenotypes were observed and recorded during the experiments. The phenotypes include heading date, leaf rolling degree, drought sensitivity and drought tolerance. Special attention was paid to leaf rolling degree at noontime. At the end of the growing season, six representative plants of each transgenic line were harvested from the middle of the row per line, and grain yield per plant was measured. The grain yield data were statistically analyzed using mixed linear model.

(b) grain yield under well-watered conditions—germinated seeds were planted in a seedbed field. At 3-leaf stage, the seedlings were transplanted into the testing field with 4 replicates and 40 plants per replicate for each transgenic line, and the 4 replicates were planted in the same block. ZH11-TC, DP0158 and negative seedlings were nearby the transgenic lines in the same block, and were used as controls in the statistical analysis. The rice plants were managed by normal practice using pesticides and fertilizers. At the end of the growing season, representative plants of each transgenic line were harvested from the middle of the row per line, and grain yield per plant was measured. The grain yield data were statistically analyzed using mixed linear model.

At the end of the season, six representative plants of each transgenic line were harvested from the middle of the row per line and grain yield per plant was measured. The grain yield per plant data were statistically analyzed using mixed linear model by ASReml program. Positive transgenic lines are selected based on the analysis (P<0.1).

The results from these studies are provided in Table 4, which provides the combined data of the transgenic lines for each of the constructs.

TABLE 4
Agronomic Characteristics of the Transgenic Rice Plants

| | | Average yield per plant | |
|---|---|---|---|
| | | under field drought | |
| No | Construct ID | conditions (g/plant) | P value |
| 1 | ZH11-TC | 7.84 ± 1.60 | 0.011^{a} |
| | DP0158 | 6.72 ± 1.60 | 0.065^{b} |
| | DP0623 | 3.86 ± 1.24 | |
| 2 | ZH11-TC | 20.52 ± 2.58 | 0.000^{a} |
| | DP0158 | 18.20 ± 2.58 | 0.000^{b} |
| | DP1804 | 7.39 ± 2.09 | |
| 3 | ZH11-TC | 19.02 ± 3.61 | 0.072^{a} |
| | DP0158 | 18.70 ± 3.28 | 0.056^{b} |
| | DP1365 | 12.81 ± 2.95 | |
| 4 | ZH11-TC | 11,51 ± 1.59 | 0.000^{a} |
| | DP0158 | 9.67 ± 1.60 | 0.000^{b} |
| | DP2253 | 1,66 ± 1.17 | |
| 5 | ZH11-TC | 5.58 ± 0.83 | 0.000^{a} |
| | DP0158 | 5.04 ± 0.83 | 0.002^{b} |
| | DP1139 | 2.80 ± 0.68 | |
| 6 | ZH11-TC | 3.89 ± 1.90 | 0.006^{a} |
| | DP0158 | 2.52 ± 1.91 | 0.031^{b} |
| | DP1448 | 1.03 ± 1.63 | |
| 7 | ZH11-TC | 4.63 ± 0.94 | 0.013^{a} |
| | DP0158 | 3.59 ± 1.05 | 0.148^{b} |
| | DP0865Y | 1.83 ± 0.91 | |
| 8 | ZH11-TC | 4.54 ± 0.60 | 0.000^{a} |
| | DP0158Y | 3.19 ± 0.59 | 0.016^{b} |
| | DP0997Y | 1.49 ± 0.45 | |
| 9 | ZH11-TC | 2.36 ± 0.56 | 0.000^{a} |
| | DP0158 | 2.47 ± 0.77 | 0.004^{b} |
| | DP0908 | 0.88 ± 0.63 | |
| 10 | ZH11-TC | 23.47 ± 1.06 | 0.000^{a} |
| | DP0158 | 23.29 ± 1.05 | 0.000^{b} |
| | DP0925 | 6.97 ± 1.51 | |

^{a}P value of the construct compared to ZH11-TC.
^{b}P value of the construct compared to DP0158.

DP0623-transgenic rice plants were tested four times in a Hainan and a Ningxia field in two years, respectively. All the tests showed that the average yield per plant of DP0623-transgenic rice decreased under field drought conditions compared to the controls; and leaf rolling and leaf necrosis phenotypes were observed in the high-expressing lines, while the low-expressing lines showed good seed setting rate without the leaf rolling phenotype. These results demonstrated that the yield and drought sensitive phenotype of DP0623-transgenic plants are correlated to the OsDN-DRT20 gene expression level. As shown in Table 4, in the Ningxia field, 9 of 12 events showed the yield per plant significantly decrease (P<0.1) compared to both controls. The average yield per plant is 51% and 43% lower than that of ZH11-TC and DP0158 control, respectively. Both yield and phenotypical observations consistently showed that OsDN-DRT20 is a rice drought sensitive gene.

DP1804-transgenic rice plants were tested two times in a Hainan and a Ningxia field in two years. All experiments consistently showed that the average yield per plant of DP1804-transgenic rice decreased, and the leaf rolling and leaf necrosis phenotypes were observed in OsEIN3-1 high-expressing lines under field drought conditions. From the Ningxia field, all 13 tested lines showed significantly decreased yield per plant than that of ZH11-TC and DP0158 controls. The average yield per plant of these 13 events is 64% and 59% lower than that of ZH11-TC and DP0158 control, respectively (Table 4). Both yield and phenotypical observations consistently showed that OsEIN3-1 is a rice drought sensitive gene.

DP1365-transgenic rice plants were tested two times in a Hainan and a Ningxia field in two years. All the experiments of testing 7 events consistently showed that the average yield per plant of OsCYP-1 lines significantly decreased compared to the controls, and the leaf rolling and leaf necrosis phenotypes were observed in OsCYP-1 lines under field drought conditions. The average yield of these 7 events is 33% and 31% lower than that of ZH11-TC and DP0158 controls, respectively. Both yield and phenotypical observations consistently showed that OsCYP-1 is a rice drought sensitive gene.

DP2253-transgenic rice plants were tested two times in a Hainan and a Ningxia field in one year. All the experiments of testing 14 events consistently showed that overexpressing of OsNAC67-3 gene in DP2253-transgenic lines significantly decreased the yield per plant compared to the controls; and leaf rolling and leaf necrosis phenotype were observed under field drought conditions. From the Ningxia field experiment, the average yield per plant of these 14 lines is 86% and 83% lower than that of ZH11-TC and DP0158 controls, respectively (Table 4). Both yield and phenotypical observations consistently showed that OsNAC67-3 is a rice drought sensitive gene.

DP1139-transgenic rice plants were tested two times under field drought conditions in a Hainan and a Ningxia field in one year. All the experiments of testing 14 events consistently showed that over-expressing of OsDN-DTP21 gene in DP1139-transgenic lines significantly decreased the yield per plant compared to the controls, and leaf rolling and leaf necrosis phenotype were observed under field drought conditions. The results for the Ningxia field experiments showed that the average yield per plant of these 14 events is 50% and 44% lower than that of ZH11-TC and DP0158 controls, respectively (Table 4). These data consistently showed that OsDN-DTP21 is a rice drought sensitive gene.

DP1448-transgenic rice plants were tested two times in one year in a Hainan and a Ningxia field, respectively. Both experiments of testing 13 events consistently showed that over-expressing of OsSIP1 significantly decreased the average yield per plant, and leaf rolling and leaf necrosis phenotype were observed under field drought conditions. From the Ningxia field experiment, the average yield per plant of these 13 lines is 74% and 59% lower than that of ZH11-TC and DP0158 controls, respectively (Table 4). These data consistently showed that OsSIP1 is a rice drought sensitive gene.

DP0865Y-transgenic rice plants were tested four times in three years in a Hainan and a Ningxia fields. All experiments consistently showed that over-expressing of OsDC1D1 gene significantly decreased the yield per plant under field drought conditions, and leaf rolling and leaf necrosis phenotype were observed under field drought conditions. From the Hainan field, 4 of 7 lines showed significantly decreased the yield per plant than that of ZH11-TC and DP0158 controls, respectively. The average yield of these 7 lines is 60% and 49% lower than that of ZH11-TC and DP0158 controls, respectively, as shown in Table 4. These data consistently showed that OsDC1D1 is a rice drought sensitive gene.

DP0997Y-transgenic rice plants were tested two times in one year in a Hainan and a Ningxia field, respectively. Both experiments of testing 12 events consistently showed that over-expressing of OsTNS1 significantly decreased the average yield per plant, and leaf rolling and leaf necrosis phenotype were observed under field drought conditions. From the Hainan field experiment, the average yield per plant of these 12 lines is 75% and 53% lower than that of ZH11-TC and DP0158 controls, respectively (Table 4). These data consistently showed that OsTNS1 is a rice drought sensitive gene.

DP0908-transgenic rice plants were tested two times in one year in a Hainan and a Ningxia field, respectively. Both experiments of testing 12 events consistently showed that over-expressing of OsSAUR27 significantly decreased the average yield per plant, and leaf rolling and leaf necrosis phenotype were observed under field drought conditions. From the Hainan field experiment, the average yield per plant of these 12 lines is 63% and 64% lower than that of ZH11-TC and DP0158 controls, respectively (Table 4). These data consistently showed that OsSAUR27 is a rice drought sensitive gene.

DP0925-transgenic rice plants were tested two times in one year in a Hainan and a Ningxia field, respectively. Both experiments of testing 12 events consistently showed that over-expressing of OsHIP1 significantly decreased the average yield per plant, and leaf rolling and leaf necrosis phenotype were observed under field drought conditions. From the Ningxia field experiment, the average yield per plant of these 12 lines is 70% and 70% lower than that of ZH11-TC and DP0158 controls, respectively (Table 4). These data consistently showed that OsHIP1 is a rice drought sensitive gene.

Taken together, these results indicate that DN-DRT20, EIN3-1, CYP-1, NAC67-3, DN-DTP21, SIP1, DC1D1, TNS1, SAUR27, or HIP1 transgenic rice plants showed drought sensitive phenotypes at the vegetative stages and produced less grain yield per plant than that of controls after drought stress.

### Example 4

### Transformation and Evaluation of Maize with Decreased Expression of the Homolog of the Rice Drought Sensitive Genes

Maize plants can be modified (e.g., suppression DNA construct or targeted genetic modification), as described herein, to reduce the expression and/or activity of the homolog from maize. Expression of the suppression elements in the maize transformation vector can be under control of a constitutive promoter such as the maize ubiquitin promoter (Christensen et al. (1989) Plant Mol. Biol. 12:619-632 and Christensen et al. (1992) Plant Mol. Biol. 18:675-689) or under control of another promoter, such as a stress-responsive promoter or a tissue-preferred promoter. The suppression DNA construct can be introduced into maize cells by particle bombardment substantially as described in International Patent Publication WO 2009/006276. Alternatively, maize plants can be transformed with the suppression DNA construct by Agrobacterium-mediated transformation substantially as described by Zhao et al. in Meth. Mol. Biol. 318:315-323 (2006) and in Zhao et al., Mol. Breed. 8:323-333 (2001) and U.S. Pat. No. 5,981,840 issued Nov. 9, 1999. Alternatively, a targeted genetic modification can be introduced at a genomic locus encoding the homologous protein using methods known in the art.

Progeny of the regenerated plants, such as T₁ plants, can be subjected to a soil-based drought stress. Using image analysis, plant area, volume, growth rate and color can be measured at multiple times before and during drought stress. Significant delay in wilting or leaf area reduction, a reduced yellow-color accumulation, and/or an increased growth rate during drought stress, relative to a control, will be considered evidence that the gene functions in maize to enhance drought tolerance.

### Example 5

### Evaluation of Sorghum with Decreased Expression of the Homolog of the Rice Drought Sensitive Genes

Sorghum can be modified (e.g., suppression DNA construct or targeted genetic modification), as described herein, to reduce the expression and/or activity of the homolog from sorghum.

Progeny of the regenerated plants, such as T₁ plants, can be subjected to a soil-based drought stress. Using image analysis, plant area, volume, growth rate and color can be measured at multiple times before and during drought stress. Significant delay in wilting or leaf area reduction, a reduced yellow-color accumulation, and/or an increased growth rate during drought stress, relative to a control, will be considered evidence that the gene functions in maize to enhance drought tolerance.

### Example 6

### Evaluation of Soybean with Decreased Expression of the Homolog of the Rice Drought Sensitive Genes

Soybean plants can be modified (e.g., suppression DNA construct or targeted genetic modification), as described herein, to reduce the expression and/or activity of the homolog from Glycine max.

Progeny of the regenerated plants, such as T₁ plants, can be subjected to a soil-based drought stress. Using image analysis, plant area, volume, growth rate and color can be measured at multiple times before and during drought stress. Significant delay in wilting or leaf area reduction, a reduced yellow-color accumulation, and/or an increased growth rate during drought stress, relative to a control, will be considered evidence that the gene functions in maize to enhance drought tolerance.

### Example 7

### Laboratory Drought Screening of Rice Drought Sensitive Genes in Arabidopsis

To understand whether rice drought tolerance genes can improve dicot plants' drought tolerance, or other traits, the rice vectors described herein can be transformed into Arabidopsis (Columbia) using floral dip method by Agrobacterium mediated transformation procedure and transgenic plants were identified (Clough, S. T. and Bent, A. F. (1998) The Plant Journal 16, 735-743; Zhang, X. et al. (2006) Nature Protocols 1: 641-646).

Progeny of the regenerated plants, such as T₁ plants, can be subjected to a soil-based drought stress. Using image analysis, plant area, volume, growth rate and color can be measured at multiple times before and during drought stress. Significant delay in wilting or leaf area reduction, a reduced yellow-color accumulation, and/or an increased growth rate during drought stress, relative to a control, will be considered evidence that the gene functions in dicot plants to enhance drought tolerance.

1-4. (canceled)

5. A modified plant or seed comprising decreased expression or activity of an endogenous polypeptide comprising an amino acid sequence having at least 80% sequence identity to any one of SEQ ID NOs: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104,106, 108, 110, 112, 114, 116, 118, 120, 122, 124, 126, 128, 130, 132, 134, 136, 138, 140, 142, 144, 146, 148, 150, or 152 when compared to the expression or activity of the corresponding polypeptide in a control plant, wherein the plant exhibits increased drought tolerance and/or grain yield.

6. (canceled)

7. The modified plant or seed of claim 5, wherein the plant comprises a suppression DNA construct comprising at least one regulatory element operably linked to the suppression elements, wherein the suppression elements comprise at least 100 contiguous base pairs of a polynucleotide with nucleotide sequence of at least 90% identity to SEQ ID NO: 1, 2, 4, 5, 7, 8, 10, 11, 13, 14, 16, 17, 19, 20, 22, 23, 25, 26, 28, 29, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151.

8. The modified plant or seed of claim 7, wherein the suppression elements comprise a nucleotide sequence of SEQ ID NO: 1, 2, 4, 5, 7, 8, 10, 11, 13, 14, 16, 17, 19, 20, 22, 23, 25, 26, 28, 29, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151.

9. The modified plant or seed of claim 5, wherein the plant comprises a targeted genetic modification at a genomic locus comprising a polynucleotide sequence encoding a polypeptide with an amino acid sequence of at least 80% sequence identity to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152, thereby decreasing expression of the polypeptide.

10. The plant of claim 5, wherein said plant is selected from the group consisting of rice, maize, soybean, sunflower, sorghum, canola, wheat, alfalfa, cotton, barley, millet, sugar cane and switchgrass.

11-17. (canceled)

18. A method of increasing drought tolerance in a plant, comprising decreasing the expression and/or activity of a polypeptide comprising an amino acid sequence having at least 80% sequence identity to any one of SEQ ID NOs: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102,104, 106, 108, 110,112, 114, 116, 118, 120, 122, 124, 126, 128, 130, 132, 134, 136, 138, 140, 142, 144, 146, 148, 150, or 152 in the plant.

19. (canceled)

20. The method of claim 18, wherein the method comprises:
(a) introducing into a regenerable plant cell a suppression DNA construct to reduce the expression or activity of the polypeptide; and
(b) regenerating a modified plant from the regenerable plant cell, wherein the plant comprises the suppression DNA construct.

21. The method of claim 20, wherein the suppression DNA construct comprises at least one heterologous regulatory element operably linked to suppression elements, wherein the suppression elements comprise at least 100 contiguous base pairs of a polynucleotide with a nucleotide sequence of at least 85% sequence identity to SEQ ID NO: 1, 2, 4, 5, 7, 8, 10, 11, 13, 14, 16, 17, 19, 20, 22, 23, 25, 26, 28, 29, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151.

22. The method of the claim 21, wherein the suppression elements comprise a polynucleotide with a nucleotide sequence of SEQ ID NO: 1, 2, 4, 5, 7, 8, 10, 11, 13, 14, 16, 17, 19, 20, 22, 23, 25, 26, 28, 29, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151.

23. The method of claim 21, wherein the heterologous regulatory element is a promoter.

24. The method of claim 19, wherein the method comprises:
(a) introducing in a regenerable plant cell a targeted genetic modification at a genomic locus that encodes the polypeptide; and
(b) generating the plant, wherein the level and/or activity of the polypeptide is decreased in the plant.

25. The method of claim 24, wherein the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), engineered site-specific meganucleases, or Argonaute.

26. The method of claim 24, wherein the targeted genetic modification is present in (a) the coding region; (b) a non-coding region; (c) a regulatory sequence; (d) an untranslated region; or (e) any combination of (a)-(d) of the genomic locus that encodes the polypeptide.

27. The method of claim 18, wherein said plant is selected from the group consisting of rice, maize, soybean, sunflower, sorghum, canola, wheat, alfalfa, cotton, barley, millet, sugar cane and switchgrass.

28. The method of claim 24, wherein the targeted genetic modification comprises introducing one or more nucleotide, replacing one or more nucleotides, or deleting one or more nucleotides in the genomic region comprising the sequence with an amino acid sequence of at least 90% sequence identity to SEQ ID NO: 3, 6, 9, 12, 15, 18, 21, 24, 27, 30, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152.


SEQUENCE LISTING (152 sequences)

SEQ ID NO: 1 - DNA, 3037 bp, Oryza sativa
gtcttctcccttgccatctctgcgctccgatccttggttgagctgctgtcttgtttgtttggctggatcaataatgcatgcgtccgtggggaggagatgggggtttgatccttgcgacgaggtaagtgttcttcagctttctcgttgcatgatgcatatatactgctttgtttctccagcttgtttgatcagtagaatcgatgtagtatcttgtatttttgttgtgatcttgctcaaaattaatatgggtattggccctccaagaaagaacttatggttattgtttggttgttaacttgttatgcttgaagttcttttttttttttgagaacaattatgcttgaagttcatgtacggaaacggagtcgtatgtactctgtatagttcagtacagtagttggtatggactggagttgatgctgctgactttcattttgatctactacatctgatctgcgaactagatgagagtcatcaggaaaaattgaagaaataataactaaccgtttaacagtacagagaaaaaatatcatactactagtacagaaagattggtactgctataaatttcttactgttgaactgtaatgttctagtagcttttctggactactaaagatgaagcgatttttttctcaggtcacccacgttgttatgaatgactttgctctgatgaatgaccacaccgtgctgcttcaaggccatgacaagtcaaggatcagcccagctggttatttgacaaggtcaggacctacacagggcggtggcatcagaaacaatattggatattgtgacgggagatccatcaatgaatcctgcggtaaaagaagcacttatccaccaacttacaagaaagatgtcactgttccaaaaagtacgcaatcttgcttgtatctttcctttttgataatggagcttttatgatggatagttctagtctaagaccatgtctatgaaatgcattcattcaggtacaaaaccaagcatttttgatgctgatgaatatgtcagtgttagcaatgtttcagacgttccttcgtcagaaggcaatactatgcaggatgagcacaggaacaaagggaaagatttgttatactgtgattggtctgaactgctcaacttggatgacctcgaagcagatctgaggtaatctcttgctgcctccagcatctatctttatccatcagaagctgagtcctttatgtgaatcttgaacatttttctttcatttttttctgaagaagtttcgagtccacgtttgagataggaagtaatcactttgaagatccactgtggtcttcagtttgcttaccagatgcccagctagtaccaagcagctgtctcttggacaataccaatttgtcaactgtttcgaatgagagcacaacaaagtctatattatcatcagtttcagtttccgatactactagtgctgaaccattgttccttgatcaggtacttccaaacttttgatctttctaattgatttccccctaattttataggttagatgatgccgagggtttcttccccagggtagcttctcttgtcattccgagagtcaagatctctggcctcttcaatatgcaacaaatctccagatcatatgtttcctcaaaataatcttggttccatataggaacattttaacatttctcttcaatttatctctgcagaataatatggcaaatcctatcaacatacaacaaccacccagcaaaggaagaagttcggcaactttgaatcatgaagcacttgcctgttcttccggggaaatcgagcgattttcacaacattcagatgttgatgttttctacccatttgacaatgtaacaagctcggaacgcataagtggctgtgagggactagaggctatcttttgcacaaatcaggaaatgctagccccaacaacatcaagcatcatgtgtgatgatgaaattgtatcttcatcgactttctcagcaccggatctcgttgcaacctacgttccgcgttcgatgaagagatctcatgatccactgaatggaactccagacatgatcctcgacgaaatggctggaaatccactagagatgtatttccctccatcattgactgcatatgaacacccagaacatctgaataacgttactttgacacaaacacaccagtttcctgaaggatttgcaggtgacgatgttctgaaaagtgcagacttacagttcctctcgaagggaaagacttcagcagacttatgtgtgaacccttgctcaccactgattctagaagctgtgccagttaaggatcttggcttccataagcttcaggaaggcatgaatcaggtatactaatagtatcagtaacttagaacaccctcttgcaacccttctagctatgttgcattcttcaggaatttgtgaatgcaattagctattattcaggttgataatatttcagattgtccatgataagtattcagctcttgtatccttccaatgtactttgcagttggacgtggcatccaaagctcgcataagagatgccttgtatcgattggccaattgtgttgagcataggcatcgcattgctagtacaacagagaccgttaaccaacttggagttatggaatcatcagcttcaaagaggtacaacggtttaatgtgatcttcaattatgtttaggacacttgaagtgatcgacagctgagtttttgtgaagtgtaattttctgacatgttcaaatcaaacaggtggagagaaattcagatgatgaaccctatggatcgctcagtggcacagctgcttctccagaaaccgctccaccataaatctccacctgattcggcgctcggcattggtccctgaattgtactgcaccgtgaaaaacacgtaggagtggctgctgatgcgatgtggtttttttttactgcacatgtctgatcgattgagcattctaggtccggcaattttttttccctctcggttccggtacgcatgtatatacagtaatcactaaagagtacagacttactagatgaaatgtaatgtgatagcaacgc

SEQ ID NO: 2 - DNA, 1569 bp, Oryza sativa
atgcatgcgtccgtggggaggagatgggggtttgatccttgcgacgaggtcacccacgttgttatgaatgactttgctctgatgaatgaccacaccgtgctgcttcaaggccatgacaagtcaaggatcagcccagctggttatttgacaaggtcaggacctacacagggcggtggcatcagaaacaatattggatattgtgacgggagatccatcaatgaatcctgcggtaaaagaagcacttatccaccaacttacaagaaagatgtcactgttccaaaaagtacaaaaccaagcatttttgatgctgatgaatatgtcagtgttagcaatgtttcagacgttccttcgtcagaaggcaatactatgcaggatgagcacaggaacaaagggaaagatttgttatactgtgattggtctgaactgctcaacttggatgacctcgaagcagatctgagaagtttcgagtccacgtttgagataggaagtaatcactttgaagatccactgtggtcttcagtttgcttaccagatgcccagctagtaccaagcagctgtctcttggacaataccaatttgtcaactgtttcgaatgagagcacaacaaagtctatattatcatcagtttcagtttccgatactactagtgctgaaccattgttccttgatcagaataatatggcaaatcctatcaacatacaacaaccacccagcaaaggaagaagttcggcaactttgaatcatgaagcacttgcctgttcttccggggaaatcgagcgattttcacaacattcagatgttgatgttttctacccatttgacaatgtaacaagctcggaacgcataagtggctgtgagggactagaggctatcttttgcacaaatcaggaaatgctagccccaacaacatcaagcatcatgtgtgatgatgaaattgtatcttcatcgactttctcagcaccggatctcgttgcaacctacgttccgcgttcgatgaagagatctcatgatccactgaatggaactccagacatgatcctcgacgaaatggctggaaatccactagagatgtatttccctccatcattgactgcatatgaacacccagaacatctgaataacgttactttgacacaaacacaccagtttcctgaaggatttgcaggtgacgatgttctgaaaagtgcagacttacagttcctctcgaagggaaagacttcagcagacttatgtgtgaacccttgctcaccactgattctagaagctgtgccagttaaggatcttggcttccataagcttcaggaaggcatgaatcagttggacgtggcatccaaagctcgcataagagatgccttgtatcgattggccaattgtgttgagcataggcatcgcattgctagtacaacagagaccgttaaccaacttggagttatggaatcatcagcttcaaagaggtggagagaaattcagatgatgaaccctatggatcgctcagtggcacagctgcttctccagaaaccgctccaccataaatctccacctgattcggcgctcggcattggtccctga

SEQ ID NO: 3 - protein, 522 aa, Oryza sativa
MHASVGRRWGFDPCDEVTHVVMNDFALMNDHTVLLQGHDKSRISPAGYLTRSGPTQGGGIRNNIGYCDGRSINESCGKRSTYPPTYKKDVTVPKSTKPSIFDADEYVSVSNVSDVPSSEGNTMQDEHRNKGKDLLYCDWSELLNLDDLEADLRSFESTFEIGSNHFEDPLWSSVCLPDAQLVPSSCLLDNTNLSTVSNESTTKSILSSVSVSDTTSAEPLFLDQNNMANPINIQQPPSKGRSSATLNHEALACSSGEIERFSQHSDVDVFYPFDNVTSSERISGCEGLEAIFCTNQEMLAPTTSSIMCDDEIVSSSTFSAPDLVATYVPRSMKRSHDPLNGTPDMILDEMAGNPLEMYFPPSLTAYEHPEHLNNVTLTQTHQFPEGFAGDDVLKSADLQFLSKGKTSADLCVNPCSPLILEAVPVKDLGFHKLQEGMNQLDVASKARIRDALYRLANCVEHRHRIASTTETVNQLGVMESSASKRWREIQMMNPMDRSVAQLLLQKPLHHKSPPDSALGIGP

SEQ ID NO: 4 - DNA, 1923 bp, Oryza sativa
atgggaggtggtctggtgatggaccagggcatgatgttccccggcgtgcacaacttcgtggatctcctgcagcagaacggcggcgacaagaacctcggcttcggcgcgctcgtgccgcagacgtcgtcgggggagcagtgcgtgatgggggagggcgacctcgtggacccgccgccggagagcttcccggacgccggtgaggacgacagcgacgacgacgtggaggacatcgaggagctggagcgccgcatgtggcgcgaccgcatgaagctgaagcggctcaaggagctgcagctgagccggggcaaggaccccgcgggcggcgtcgtgggcgacccgtccaagccgcggcagtcgcaggagcaggcgcggcggaagaagatgtcgcgcgcgcaggacggcatcctcaagtacatgctcaagatgatggaggtgtgccgcgcgcaggggttcgtgtacgggatcatcccggagaagggcaagccggtgagcggcgcctccgacaacctccgcggctggtggaaggagaaggtccgcttcgaccgcaacggccccgccgccatcgccaagtaccaggccgacaacgccgtcccgggcttcgagagcgagctcgcctccggcaccgggagcccgcactcgctgcaggagctgcaggacaccaccctcgggtcgctgctctcggcgctcatgcagcactgcgaccctccgcagcggcggtacccgctcgagaagggcgtccctccgccgtggtggcccaccggcgacgaggagtggtggccggagctcggcatccccaaggaccagggcccgcctccgtacaagaagccccatgacctcaagaaggcctggaaggtcagcgtgctcaccgctgtcatcaagcacatgtcgccggacatcgagaagatccgccggctggtccggcagtccaagtgcctccaggacaagatgaccgccaaggagatctccacctggctggccgtcgtcaagcaggaagaggagctgtacctgaagctgaaccccggtgcccgccctccggcacctaccggcggcatcaccagcgccatatcgttcaacgccagctcaagtgagtacgacgtcgacgtcgtcgacgactgcaagggcgacgaggccggcaaccagaaggctgttgttgtcgccgacccgaccgcgttcaacctcggcgcggctatgctgaacgacaagttcctcatgccggcgtccatgaaggaggaggccaccgatgtcgagttcatccagaagaggagcgcgtctggcgcggagcctgagctgatgctgaacaaccgtgtctacacctgccacaatgtccagtgcccgcatagcgactatggatacgggttccttgaccggaacgcgcgcaacagccaccaatacacttgcaagtacaatgatccactccagcagagcacggagaacaagccatcgccaccggccatcttcccggcaacctacaacacgccgaaccaggctctgaacaatctggatttcggcctgcccatggatggccagaggtcaattacagagctgatgaacatgtacgacaacaacttcgtggccaacaagaaccttagcaacgacaatgccacgatcatggagaggcctaatgcagtcaacccaaggatacagattgaagaaggcttttttggacagggaagtggcatcggcggcagcaacggaggtgtgttcgaagatgtcaatggcatgatgcagcaaccgcagcagaccaccccggcacagcagcagttcttcatccgcgacgatactccattcggtaaccagatgggcgacatcaatggcgcatcggagttcaggttcggctctggtttcaacatgtcaggtgccgtcgaataccccggcgcaatgcagggccagcagaagaatgacggctcgaattggtactactga

SEQ ID NO: 5 - DNA, 1923 bp, Oryza sativa; identical to SEQ ID NO: 4

SEQ ID NO: 6 - protein, 640 aa, Oryza sativa
MGGGLVMDQGMMFPGVHNFVDLLQQNGGDKNLGFGALVPQTSSGEQCVMGEGDLVDPPPESFPDAGEDDSDDDVEDIEELERRMWRDRMKLKRLKELQLSRGKDPAGGVVGDPSKPRQSQEQARRKKMSRAQDGILKYMLKMMEVCRAQGFVYGIIPEKGKPVSGASDNLRGWWKEKVRFDRNGPAAIAKYQADNAVPGFESELASGTGSPHSLQELQDTTLGSLLSALMQHCDPPQRRYPLEKGVPPPWWPTGDEEWWPELGIPKDQGPPPYKKPHDLKKAWKVSVLTAVIKHMSPDIEKIRRLVRQSKCLQDKMTAKEISTWLAVVKQEEELYLKLNPGARPPAPTGGITSAISFNASSSEYDVDVVDDCKGDEAGNQKAVVVADPTAFNLGAAMLNDKFLMPASMKEEATDVEFIQKRSASGAEPELMLNNRVYTCHNVQCPHSDYGYGFLDRNARNSHQYTCKYNDPLQQSTENKPSPPAIFPATYNTPNQALNNLDFGLPMDGQRSITELMNMYDNNFVANKNLSNDNATIMERPNAVNPRIQIEEGFFGQGSGIGGSNGGVFEDVNGMMQQPQQTTPAQQQFFIRDDTPFGNQMGDINGASEFRFGSGFNMSGAVEYPGAMQGQQKNDGSNWYY

SEQ ID NO: 7 - DNA, 1457 bp, Oryza sativa
gacaaagataagtgaagtgagcaggcgccaatgggtgcttttcttctgttcgtgtgcgtgctcgcgcctttcttgcttgtctgcgccgtccgcggccgccgccggcaggcgggctcgtcggaagcggcggcgtgcggcctgccgctgccgccggggtcgatggggtggccgtacgtcggggagacgttccagctgtactcgtccaagaaccccaacgtgttcttcaacaagaagcggaacaagtacggtcccatcttcaagacgcacatcctgggatgcccctgcgtgatggtgtccagcccggaggcggcgcggttcgtgctggtgacgcaggcgcacctcttcaagcccaccttcccggcgagcaaggagcggatgctgggtccccaggccatcttcttccagcagggcgactaccacgcccacctccgccgcatcgtctcccgcgccttctcccccgagtccatccgcgcctccgtcccggccatcgaggccatcgcgctccgctccctccactcctgggacggccagttcgtcaacaccttccaagagatgaagacttacgcgctgaatgtggcattgctgtccatcttcggggaggaggagatgcgctacatcgaggagctgaagcagtgctacctgacgctggagaaggggtacaactcgatgccggtgaacctgccgggcaccctgttccacaaggccatgaaggcccggaagaggctgggcgccattgtggcccacatcatctctgcccggcgcgagcggcagcgggggaacgacctgctagggtcgttcgtggacggccgcgaggccctcaccgacgcccagatcgccgacaacgtcatcggcgtcatcttcgccgcccgcgacaccaccgccagcgtcctcacctggatggtcaagttcctcggcgaccaccccgccgtcctcaaggccgtcaccgaagagcagctgcagattgccaaggagaaagaggcgtcgggcgagccgctgtcatgggcggacacgcggcggatgaagatgacgagccgggtcatccaggagacgatgagggtggcgtccatcctctccttcaccttcagggaggccgtggaggacgtggaataccaagggtacctgatccccaagggctggaaagtgctacctctgttccgcaacatccaccacaaccccgaccacttcccctgcccggaaaagttcgacccgtcccggttcgaggtggcgcccaagcccaacacgttcatgccgttcgggaacgggacccactcgtgcccgggcaacgagctcgccaagctggagatgctcgtgctcttccaccacctcgcaaccaagtacaggtggtccacgtccaagtccgagagcggcgtccagttcggccccttcgcgctgccgctcaacggcctccccatgagcttcacccgcaagaacaccgagcaggagtgaaaaccgaacag

SEQ ID NO: 8 - DNA, 1416 bp, Oryza sativa
atgggtgcttttcttctgttcgtgtgcgtgctcgcgcctttcttgcttgtctgcgccgtccgcggccgccgccggcaggcgggctcgtcggaagcggcggcgtgcggcctgccgctgccgccggggtcgatggggtggccgtacgtcggggagacgttccagctgtactcgtccaagaaccccaacgtgttcttcaacaagaagcggaacaagtacggtcccatcttcaagacgcacatcctgggatgcccctgcgtgatggtgtccagcccggaggcggcgcggttcgtgctggtgacgcaggcgcacctcttcaagcccaccttcccggcgagcaaggagcggatgctgggtccccaggccatcttcttccagcagggcgactaccacgcccacctccgccgcatcgtctcccgcgccttctcccccgagtccatccgcgcctccgtcccggccatcgaggccatcgcgctccgctccctccactcctgggacggccagttcgtcaacaccttccaagagatgaagacttacgcgctgaatgtggcattgctgtccatcttcggggaggaggagatgcgctacatcgaggagctgaagcagtgctacctgacgctggagaaggggtacaactcgatgccggtgaacctgccgggcaccctgttccacaaggccatgaaggcccggaagaggctgggcgccattgtggcccacatcatctctgcccggcgcgagcggcagcgggggaacgacctgctagggtcgttcgtggacggccgcgaggccctcaccgacgcccagatcgccgacaacgtcatcggcgtcatcttcgccgcccgcgacaccaccgccagcgtcctcacctggatggtcaagttcctcggcgaccaccccgccgtcctcaaggccgtcaccgaagagcagctgcagattgccaaggagaaagaggcgtcgggcgagccgctgtcatgggcggacacgcggcggatgaagatgacgagccgggtcatccaggagacgatgagggtggcgtccatcctctccttcaccttcagggaggccgtggaggacgtggaataccaagggtacctgatccccaagggctggaaagtgctacctctgttccgcaacatccaccacaaccccgaccacttcccctgcccggaaaagttcgacccgtcccggttcgaggtggcgcccaagcccaacacgttcatgccgttcgggaacgggacccactcgtgcccgggcaacgagctcgccaagctggagatgctcgtgctcttccaccacctcgcaaccaagtacaggtggtccacgtccaagtccgagagcggcgtccagttcggccccttcgcgctgccgctcaacggcctccccatgagcttcacccgcaagaacaccgagcaggagtga

SEQ ID NO: 9 - protein, 471 aa, Oryza sativa
MGAFLLFVCVLAPFLLVCAVRGRRRQAGSSEAAACGLPLPPGSMGWPYVGETFQLYSSKNPNVFFNKKRNKYGPIFKTHILGCPCVMVSSPEAARFVLVTQAHLFKPTFPASKERMLGPQAIFFQQGDYHAHLRRIVSRAFSPESIRASVPAIEAIALRSLHSWDGQFVNTFQEMKTYALNVALLSIFGEEEMRYIEELKQCYLTLEKGYNSMPVNLPGTLFHKAMKARKRLGAIVAHIISARRERQRGNDLLGSFVDGREALTDAQIADNVIGVIFAARDTTASVLTWMVKFLGDHPAVLKAVTEEQLQIAKEKEASGEPLSWADTRRMKMTSRVIQETMRVASILSFTFREAVEDVEYQGYLIPKGWKVLPLFRNIHHNPDHFPCPEKFDPSRFEVAPKPNTFMPFGNGTHSCPGNELAKLEMLVLFHHLATKYRWSTSKSESGVQFGPFALPLNGLPMSFTRKNTEQE

SEQ ID NO: 10 - DNA, 991 bp, Oryza sativa
gccacagagagagcagtagtagtagcgagctcgccggagaacggacgatcaccggagaagggggagagagatgagcggcggtcaggacctgcagctgccgccggggttccggttccacccgacggacgaggagctggtgatgcactacctctgccgccgctgcgccggcctccccatcgccgtccccatcatcgccgagatcgacctctacaagttcgatccatggcagcttccccggatggcgctgtacggagagaaggagtggtacttcttctccccgcgagaccgcaagtacccgaacgggtcgcggccgaaccgcgccgccgggtcggggtactggaaggcgaccggcgccgacaagccggtgggctcgccgaagccggtggcgatcaagaaggccctcgtcttctacgccggcaaggcgcccaagggcgagaagaccaactggatcatgcacgagtaccgcctcgccgacgtcgaccgctccgcccgcaagaagaacagcctcaggttggatgattgggtgctgtgccggatttacaacaagaagggcgggctggagaagccgccggccgcggcggtggcggcggcggggatggtgagcagcggcggcggcgtccagaggaagccgatggtgggggtgaacgcggcggtgagctccccgccggagcagaagccggtggtggcggggccggcgttcccggacctggcggcgtactacgaccggccgtcggactcgatgccgcggctgcacgccgactcgagctgctcggagcaggtgctgtcgccggagttcgcgtgcgaggtgcagagccagcccaagatcagcgagtgggagcgcaccttcgccaccgtcgggcccatcaaccccgccgcctccatcctcgaccccgccggctccggcggcctcggcggcctcggcggcggcggcagcgaccccctcctccaggacatcctcatgtactggggcaagccattctagacgaccaaa

SEQ ID NO: 11 - DNA, 912 bp, Oryza sativa
atgagcggcggtcaggacctgcagctgccgccggggttccggttccacccgacggacgaggagctggtgatgcactacctctgccgccgctgcgccggcctccccatcgccgtccccatcatcgccgagatcgacctctacaagttcgatccatggcagcttccccggatggcgctgtacggagagaaggagtggtacttcttctccccgcgagaccgcaagtacccgaacgggtcgcggccgaaccgcgccgccgggtcggggtactggaaggcgaccggcgccgacaagccggtgggctcgccgaagccggtggcgatcaagaaggccctcgtcttctacgccggcaaggcgcccaagggcgagaagaccaactggatcatgcacgagtaccgcctcgccgacgtcgaccgctccgcccgcaagaagaacagcctcaggttggatgattgggtgctgtgccggatttacaacaagaagggcgggctggagaagccgccggccgcggcggtggcggcggcggggatggtgagcagcggcggcggcgtccagaggaagccgatggtgggggtgaacgcggcggtgagctccccgccggagcagaagccggtggtggcggggccggcgttcccggacctggcggcgtactacgaccggccgtcggactcgatgccgcggctgcacgccgactcgagctgctcggagcaggtgctgtcgccggagttcgcgtgcgaggtgcagagccagcccaagatcagcgagtgggagcgcaccttcgccaccgtcgggcccatcaaccccgccgcctccatcctcgaccccgccggctccggcggcctcggcggcctcggcggcggcggcagcgaccccctcctccaggacatcctcatgtactggggcaagccattctag

SEQ ID NO: 12 - protein, 303 aa, Oryza sativa
MSGGQDLQLPPGFRFHPTDEELVMHYLCRRCAGLPIAVPIIAEIDLYKFDPWQLPRMALYGEKEWYFFSPRDRKYPNGSRPNRAAGSGYWKATGADKPVGSPKPVAIKKALVFYAGKAPKGEKTNWIMHEYRLADVDRSARKKNSLRLDDWVLCRIYNKKGGLEKPPAAAVAAAGMVSSGGGVQRKPMVGVNAAVSSPPEQKPVVAGPAFPDLAAYYDRPSDSMPRLHADSSCSEQVLSPEFACEVQSQPKISEWERTFATVGPINPAASILDPAGSGGLGGLGGGGSDPLLQDILMYWGKPF

SEQ ID NO: 13 - DNA, 2051 bp, Oryza sativa
cattgtgaccatccatccatcgatctctccgaattgagctcgctgtcgccgtcgagctggcagcaatgcgagacttctcctgcttcggcgacggcgccgtcagcctcgcggcctcagctgccgcggccggggcgggcgccgcgctcgaccgctcgctgcaggcggccacggcgaccgtctacaaggccgccttgtcttcgcgcaaggagatcctcgtcagggtcatgtggaccaggaccgtcgccggtgccgcgcccggcggtgctactggcctcgccgtcgccgtcgacgaggcctcccggtcgtccccctcgcccgctgctggttcagcttcagcagcgacgcctcgccggtcggccgtcgcgctggcgagctcgccgcagttcctgcacaagaagcgcgggacccggtcgttcgtcaccgaggccggcacggtggtggccatctactgggacaccacggacgccaagtaccccgccgccgggtcgtcgtccccggagccgacgcgcgactactacctcgccgtcgtcgccgacggggagctcgcggtcctcctgggcgggggcgaggcggcgcgggagctcgcacgccgcttcgccgctgcgccgcggcgcgcgttgctaagccggcgggagcagctccgcgcggcgccggcttccccggcggcgatggcggcggcggcggtggcgcacagcacgcggtgcaggttccgggctgacggggcggagcacgaggtggcggtggtgtgccgcggggaggagtgggggactcgggacggggaggtggcggtgagcatcgacgggaagaaggtggtggaggcgcggcgggtgaagtggaacttccgggggaacaggacggcggtgctcggggacggcgcggtggtggaggtgatgtgggacgtgcacgactggtggttcgccggcggcggcggcggcggggcgcagttcatggtgaaggcgcgcgacggagacggagacggagacggcgggagggtgtggatggacgaggtgatggccagcaagggccatcctcccggaggattcttcctgcacgtccaatgctaccgccggtgatgatcggcggcgctggttagagagatgtcaatggtgtgtgatggtcgcattcgcatcgcagtaatgatgattctttaattaatctagtatcaaactttgcgactttgttaagggacttgagatgaacttatttcctttgtatcttagcaggccggatgtatgggctggaatagaggcccatgcatcaattaaggccgcggtagagagaagcccgccacttcccctttcggacgccgtccgactcgattccgattccgattgcgattgaagctctgttacttcgacgaagtagtcggcgagggatcggtgaacctgttacctatgccgaggatgaggcggatgaggaggaggtcatcgtgtgcggcgatgatctggggctccatggcgaagtaattggttttagggttgagggtgggagagtggacgcacacgtaggagacgagcggcgggcaagcggcggtgcacaaggtgacctggatttcatggccctcccttgttttggaggtcggtggtggcgttgcggacgtcggcgtcagagaagtaggcgagcttgtcgaggagtacccatcttggcatccgccgccgccgctttctcttgccgagggagcggccgtggacaggaggatacagggaggtgtacatgatgcccatttgcgccatgatctgatcgcctcctggctttcaagtagctagcagcctactggatttatctgctatctatctgctacacatacaataaataggcggcctccattgctgatgatatcccgccgtctctcacgcgcatcacccatcccggccctatgatcaccttcactaaccacgaggtctccatcctgcactacatccctgaacctcaggacattaacgaacccctcatcagcaacaaacaacgctacatcgtcaccgcgctttctgtcaacaggtttaggccaccaggggaatacgagctccacctctacaactcccacacctagc

SEQ ID NO: 14 - DNA, 1176 bp, Oryza sativa
atgcgagacttctcctgcttcggcgacggcgccgtcagcctcgcggcctcagctgccgcggccggggcgggcgccgcgctcgaccgctcgctgcaggcggccacggcgaccgtctacaaggccgccttgtcttcgcgcaaggagatcctcgtcagggtcatgtggaccaggaccgtcgccggtgccgcgcccggcggtgctactggcctcgccgtcgccgtcgacgaggcctcccggtcgtccccctcgcccgctgctggttcagcttcagcagcgacgcctcgccggtcggccgtcgcgctggcgagctcgccgcagttcctgcacaagaagcgcgggacccggtcgttcgtcaccgaggccggcacggtggtggccatctactgggacaccacggacgccaagtaccccgccgccgggtcgtcgtccccggagccgacgcgcgactactacctcgccgtcgtcgccgacggggagctcgcggtcctcctgggcgggggcgaggcggcgcgggagctcgcacgccgcttcgccgctgcgccgcggcgcgcgttgctaagccggcgggagcagctccgcgcggcgccggcttccccggcggcgatggcggcggcggcggtggcgcacagcacgcggtgcaggttccgggctgacggggcggagcacgaggtggcggtggtgtgccgcggggaggagtgggggactcgggacggggaggtggcggtgagcatcgacgggaagaaggtggtggaggcgcggcgggtgaagtggaacttccgggggaacaggacggcggtgctcggggacggcgcggtggtggaggtgatgtgggacgtgcacgactggtggttcgccggcggcggcggcggcggggcgcagttcatggtgaaggcgcgcgacggagacggagacggagacggcgggagggtgtggatggacgaggcggcctccattgctgatgatatcccgccgtctctcacgcgcatcacccatcccggccctatgatcaccttcactaaccacgaggtctccatcctgcactacatccctgaacctcaggacattaacgaacccctcatcagcaacaaacaacgctacatcgtcaccgcgctttctgtcaacaggtttaggccaccaggggaatacgagctccacctctacaactcccacacctag

SEQ ID NO: 15 - protein, 391 aa, Oryza sativa
MRDFSCFGDGAVSLAASAAAAGAGAALDRSLQAATATVYKAALSSRKEILVRVMWTRTVAGAAPGGATGLAVAVDEASRSSPSPAAGSASAATPRRSAVALASSPQFLHKKRGTRSFVTEAGTVVAIYWDTTDAKYPAAGSSSPEPTRDYYLAVVADGELAVLLGGGEAARELARRFAAAPRRALLSRREQLRAAPASPAAMAAAAVAHSTRCRFRADGAEHEVAVVCRGEEWGTRDGEVAVSIDGKKVVEARRVKWNFRGNRTAVLGDGAVVEVMWDVHDWWFAGGGGGGAQFMVKARDGDGDGDGGRVWMDEAASIADDIPPSLTRITHPGPMITFTNHEVSILHYIPEPQDINEPLISNKQRYIVTALSVNRFRPPGEYELHLYNSHT

SEQ ID NO: 16 - DNA, 1470 bp, Oryza sativa
gagtggagcgatctcgatggacccgtgcccgttcgtgcgggtgctggtcggcaacctctcgctgaagatgccggtggcgccgcgccccgccggagccggggccggggtgcacccatccacctcgccgtgctactgcaagatccgcctcaacaagctgccgtaccagaccgccgacgcgccgctgctgctgccgccctcgccggaggcatcggcggcgccggcgccagcgccggcgacgggcgcgctcgccgccgcgttccacctctccaaggccgacctcgaccgcctcaccgcgaagccgtcgctgttcgggtcgcgcacggcgaggctgaagatcgtggtgtacgctggccggaggggcaccacgtgcggcgtcggcggcggctccgggaggctgctcgggaaggtggtcatcccgctcgacctcaagggcgcctcggcgaagccggtggtgtaccacagcagctggatctgcatcgggaagcgcgggcgcaagccctcgtcggtgtcggcggcgaacgcgcagctcaacatcacggtgcgcgccgagcccgacccgaggttcgtgttcgagttcgacggcgagccggagtgcagcccgcaggtgctccaggtgcaggggagcatgaagcagcccatgttcacctgcaagttctcctgccgcagcaacagcgacctccgctcccggtcaatgccggccgatatggggagcggcgggcgcaactggctgacggcgttcggctccgacagggagcgggcggggaaggagaggaaggggtggtcggtgacggtgcacgacctgtcaggctccccggtggcgctggcatcaatggtgacgccgttcgtggcgtcgccggggacggacagggtgagcaaatccaacccgggggcgtggctggtgctccgcccgggcgacggcacgtggaagccatggggtcgcctggaatgctggcgcgagcgcggcgcgggcgccgccgccggcgacagcctcgggtaccggttcgagctcgtcctccccgacccaaccggcatgggcgtgggcgtgtccgtggcggagtccaccatcccggcgtcgaagggcggccggttcgcgatcgacctgacggcaacgcaacagttcgggcggagcgggtcgccggcgtgcagcccgtgcgggagcggcgactacgggatgtggccgttcggcagctgccgcgggttcgtgatgtcggcggcggtgcagggggaggggaaatgcagccggccggcggtggaggtgggcgtgcagaacgtcgggtgcgcggaggacgcggcggcgttcgtggcgctcgccgccgccgtcgacctgagcatggacgcgtgccggctcttctcccaccgcctccgccgcgagctctcggcgtcgcgctccgacctgctccggtgaggcacacgaggcggcggtgaatcgatcgatcgatcggaatcgggaacaacattgtacagctagcg

SEQ ID NO: 17 - DNA, 1389 bp, Oryza sativa
atggacccgtgcccgttcgtgcgggtgctggtcggcaacctctcgctgaagatgccggtggcgccgcgccccgccggagccggggccggggtgcacccatccacctcgccgtgctactgcaagatccgcctcaacaagctgccgtaccagaccgccgacgcgccgctgctgctgccgccctcgccggaggcatcggcggcgccggcgccagcgccggcgacgggcgcgctcgccgccgcgttccacctctccaaggccgacctcgaccgcctcaccgcgaagccgtcgctgttcgggtcgcgcacggcgaggctgaagatcgtggtgtacgctggccggaggggcaccacgtgcggcgtcggcggcggctccgggaggctgctcgggaaggtggtcatcccgctcgacctcaagggcgcctcggcgaagccggtggtgtaccacagcagctggatctgcatcgggaagcgcgggcgcaagccctcgtcggtgtcggcggcgaacgcgcagctcaacatcacggtgcgcgccgagcccgacccgaggttcgtgttcgagttcgacggcgagccggagtgcagcccgcaggtgctccaggtgcaggggagcatgaagcagcccatgttcacctgcaagttctcctgccgcagcaacagcgacctccgctcccggtcaatgccggccgatatggggagcggcgggcgcaactggctgacggcgttcggctccgacagggagcgggcggggaaggagaggaaggggtggtcggtgacggtgcacgacctgtcaggctccccggtggcgctggcatcaatggtgacgccgttcgtggcgtcgccggggacggacagggtgagcaaatccaacccgggggcgtggctggtgctccgcccgggcgacggcacgtggaagccatggggtcgcctggaatgctggcgcgagcgcggcgcgggcgccgccgccggcgacagcctcgggtaccggttcgagctcgtcctccccgacccaaccggcatgggcgtgggcgtgtccgtggcggagtccaccatcccggcgtcgaagggcggccggttcgcgatcgacctgacggcaacgcaacagttcgggcggagcgggtcgccggcgtgcagcccgtgcgggagcggcgactacgggatgtggccgttcggcagctgccgcgggttcgtgatgtcggcggcggtgcagggggaggggaaatgcagccggccggcggtggaggtgggcgtgcagaacgtcgggtgcgcggaggacgcggcggcgttcgtggcgctcgccgccgccgtcgacctgagcatggacgcgtgccggctcttctcccaccgcctccgccgcgagctctcggcgtcgcgctccgacctgctccggtga

SEQ ID NO: 18 - protein, 462 aa, Oryza sativa
MDPCPFVRVLVGNLSLKMPVAPRPAGAGAGVHPSTSPCYCKIRLNKLPYQTADAPLLLPPSPEASAAPAPAPATGALAAAFHLSKADLDRLTAKPSLFGSRTARLKIVVYAGRRGTTCGVGGGSGRLLGKVVIPLDLKGASAKPVVYHSSWICIGKRGRKPSSVSAANAQLNITVRAEPDPRFVFEFDGEPECSPQVLQVQGSMKQPMFTCKFSCRSNSDLRSRSMPADMGSGGRNWLTAFGSDRERAGKERKGWSVTVHDLSGSPVALASMVTPFVASPGTDRVSKSNPGAWLVLRPGDGTWKPWGRLECWRERGAGAAAGDSLGYRFELVLPDPTGMGVGVSVAESTIPASKGGRFAIDLTATQQFGRSGSPACSPCGSGDYGMWPFGSCRGFVMSAAVQGEGKCSRPAVEVGVQNVGCAEDAAAFVALAAAVDLSMDACRLFSHRLRRELSASRSDLLR

SEQ ID NO: 19 - DNA, 880 bp, Oryza sativa
gggtgaaaatatctggagaacaagctttaactaattcagctatggtgagctcgagctttcctgcggagatcatccaccctgcccgcctgggttgcatgctgaggctgcacgtggtggagcatcccaccggcgacgccgccgcggtcgccttccagtgcgacggctgcatgctacccggagaaggcacgaggtacacctccgtcgtcgacaaccacccgacacacctcgccctccacacgagctgcgccctcgcgacgcccacgctgcagcacgcgctggtgaagggcacgatggagctccgccacgaggcccccgccggcggcgccggcgtttgctccgcctgcttcgagacggtgcggggattccactactacgggtcgaggaagaccggcaagggcgagcacccgaagctgcacccgtgctgcgcgaggctgccggtgtccatcgccgtgcggggcgggctcaccttcgagcttcgcgcggaggtgtcgcaccggtgcaccggctgcagggcgatggagtggtactaccgcccttggtgctaccgctccactaatagccccgaccaccgcgtgtacctgcacgtcaagtgcatcagggagatcatggaatctccgggcggcggcggaggcggaggcgccggtgatgaagacgacagggtggtggcccgtctactggagcgcgctgaccagagcagtaagctggagaggcgcgtatgtaagatccttgtgatcttggtgcgtgtcgtcgtcaggatgctcatcggagacccgaccgcgttgttgacagaaggagtgagcgctatcgtgtctccatggtgatgctgtatatatatagcccgtgaacgcctagctagcttaaggccgtatatatgtgctcg

SEQ ID NO: 20 - DNA, 780 bp, Oryza sativa
atggtgagctcgagctttcctgcggagatcatccaccctgcccgcctgggttgcatgctgaggctgcacgtggtggagcatcccaccggcgacgccgccgcggtcgccttccagtgcgacggctgcatgctacccggagaaggcacgaggtacacctccgtcgtcgacaaccacccgacacacctcgccctccacacgagctgcgccctcgcgacgcccacgctgcagcacgcgctggtgaagggcacgatggagctccgccacgaggcccccgccggcggcgccggcgtttgctccgcctgcttcgagacggtgcggggattccactactacgggtcgaggaagaccggcaagggcgagcacccgaagctgcacccgtgctgcgcgaggctgccggtgtccatcgccgtgcggggcgggctcaccttcgagcttcgcgcggaggtgtcgcaccggtgcaccggctgcagggcgatggagtggtactaccgcccttggtgctaccgctccactaatagccccgaccaccgcgtgtacctgcacgtcaagtgcatcagggagatcatggaatctccgggcggcggcggaggcggaggcgccggtgatgaagacgacagggtggtggcccgtctactggagcgcgctgaccagagcagtaagctggagaggcgcgtatgtaagatccttgtgatcttggtgcgtgtcgtcgtcaggatgctcatcggagacccgaccgcgttgttgacagaaggagtgagcgctatcgtgtctccatggtga

SEQ ID NO: 21 - protein, 259 aa, Oryza sativa
MVSSSFPAEIIHPARLGCMLRLHVVEHPTGDAAAVAFQCDGCMLPGEGTRYTSVVDNHPTHLALHTSCALATPTLQHALVKGTMELRHEAPAGGAGVCSACFETVRGFHYYGSRKTGKGEHPKLHPCCARLPVSIAVRGGLTFELRAEVSHRCTGCRAMEWYYRPWCYRSTNSPDHRVYLHVKCIREIMESPGGGGGGGAGDEDDRVVARLLERADQSSKLERRVCKILVILVRVVVRMLIGDPTALLTEGVSAIVSPW

SEQ ID NO: 22 - DNA, 1649 bp, Oryza sativa
ccatcctcctccctaattactccccccatcccctcctcctccgccgccaagcacctcgcctcctccgccatggcgaccgccaccgcgtcgtccctctctctcctcttcgcccacccacactcgtccaaccccaggcccttcgccggcgggcctcacctccgccgcccgctgcgcgccgcgccccaccgcgcgcgatgcgcctccgacgccgccacgacggccacgaggcaccgccgccccgcggaggagaacatccgggaggaggccgcgcggctccgcggccccgggaacgacttctcggcgtggtacgtgccgttccccccgacgcccgaggacgaccccgacgagcgctactcgctggacgaggtggtctaccgctccagctccggggggctcctcgacgtgtgccacgacatggaggcgctcgcgcgcttcccgggctcctactggcgcgacctcttcgactcccgcgtggggcgcaccgcgtggccctacggctccggggtgtggtccaagaaggagttcgtgctcccggagatcgactccgaccacatcgtctccctcttcgagggcaactccaacctcttctgggcggagcgcctcggccgcgagcacctcggcgggatgaccgacctctgggttaagcactgcggcatctcgcacacgggctccttcaaggacctcggcatgacggtgctcgtcagccaggtgaaccgcctccgccgcgcgccgctctcacgccccatcaacggcgtcggctgcgcgtccacgggcgacacctccgccgcgctttccgcgtactgcgccgccgcaggtatccccgccatcgtgttcctccccgccgaccgcatctctctgcagcagctcatccagccaatcgccaacggcgccaccgtgctctcgctggacacggactttgacgggtgcatgcggctcatcagggaggtgaccgccgagctaccgatttaccttgccaactcgctgaactcccttcggcttgagggccagaagacggcagccatcgagatattgcagcagttcgattggcaggtgccggattgggtcattgttccaggaggcaatcttgggaatatctatgccttctacaaggggtttgagatgtgccgtgttcttgggcttgttgatcgtgtgccgcgtcttgtatgcgcacaagctgcaaacgcaaatccgttgtatcggttctacaagtcagggtggactgatttccagccacgtgtagccgaaactacatttgcatctgccatacagattggtgatccagtatctgtcgaccgtgcagtggtcgccctgaaggcaactgacggtattgttgaggaagctacggaggaagaactcatggatgcaatgtcacttgctgaccgcaccggaatgtttgcctgcccacacaccggggttgcacttgctgctttgttcaagcttcgagaccagcgcataatcgggcctaatgaccgcacagtggttgttagtacagcgcatgggcttaagttcacacaatcgaagatagactaccatgacaggaacatcaaggacatgctgtgccagtacgctaatccaccaatcaatgtgaaggctgactttgcttctgtgatggatgttctccagaacaagctgaatggtaagatctgagc

SEQ ID NO: 23 - DNA, 1578 bp, Oryza sativa
atggcgaccgccaccgcgtcgtccctctctctcctcttcgcccacccacactcgtccaaccccaggcccttcgccggcgggcctcacctccgccgcccgctgcgcgccgcgccccaccgcgcgcgatgcgcctccgacgccgccacgacggccacgaggcaccgccgccccgcggaggagaacatccgggaggaggccgcgcggctccgcggccccgggaacgacttctcggcgtggtacgtgccgttccccccgacgcccgaggacgaccccgacgagcgctactcgctggacgaggtggtctaccgctccagctccggggggctcctcgacgtgtgccacgacatggaggcgctcgcgcgcttcccgggctcctactggcgcgacctcttcgactcccgcgtggggcgcaccgcgtggccctacggctccggggtgtggtccaagaaggagttcgtgctcccggagatcgactccgaccacatcgtctccctcttcgagggcaactccaacctcttctgggcggagcgcctcggccgcgagcacctcggcgggatgaccgacctctgggttaagcactgcggcatctcgcacacgggctccttcaaggacctcggcatgacggtgctcgtcagccaggtgaaccgcctccgccgcgcgccgctctcacgccccatcaacggcgtcggctgcgcgtccacgggcgacacctccgccgcgctttccgcgtactgcgccgccgcaggtatccccgccatcgtgttcctccccgccgaccgcatctctctgcagcagctcatccagccaatcgccaacggcgccaccgtgctctcgctggacacggactttgacgggtgcatgcggctcatcagggaggtgaccgccgagctaccgatttaccttgccaactcgctgaactcccttcggcttgagggccagaagacggcagccatcgagatattgcagcagttcgattggcaggtgccggattgggtcattgttccaggaggcaatcttgggaatatctatgccttctacaaggggtttgagatgtgccgtgttcttgggcttgttgatcgtgtgccgcgtcttgtatgcgcacaagctgcaaacgcaaatccgttgtatcggttctacaagtcagggtggactgatttccagccacgtgtagccgaaactacatttgcatctgccatacagattggtgatccagtatctgtcgaccgtgcagtggtcgccctgaaggcaactgacggtattgttgaggaagctacggaggaagaactcatggatgcaatgtcacttgctgaccgcaccggaatgtttgcctgcccacacaccggggttgcacttgctgctttgttcaagcttcgagaccagcgcataatcgggcctaatgaccgcacagtggttgttagtacagcgcatgggcttaagttcacacaatcgaagatagactaccatgacaggaacatcaaggacatgctgtgccagtacgctaatccaccaatcaatgtgaaggctgactttgcttctgtgatggatgttctccagaacaagctgaatggtaagatctga

SEQ ID NO: 24 - protein, 525 aa, Oryza sativa
MATATASSLSLLFAHPHSSNPRPFAGGPHLRRPLRAAPHRARCASDAATTATRHRRPAEENIREEAARLRGPGNDFSAWYVPFPPTPEDDPDERYSLDEVVYRSSSGGLLDVCHDMEALARFPGSYWRDLFDSRVGRTAWPYGSGVWSKKEFVLPEIDSDHIVSLFEGNSNLFWAERLGREHLGGMTDLWVKHCGISHTGSFKDLGMTVLVSQVNRLRRAPLSRPINGVGCASTGDTSAALSAYCAAAGIPAIVFLPADRISLQQLIQPIANGATVLSLDTDFDGCMRLIREVTAELPIYLANSLNSLRLEGQKTAAIEILQQFDWQVPDWVIVPGGNLGNIYAFYKGFEMCRVLGLVDRVPRLVCAQAANANPLYRFYKSGWTDFQPRVAETTFASAIQIGDPVSVDRAVVALKATDGIVEEATEEELMDAMSLADRTGMFACPHTGVALAALFKLRDQRIIGPNDRTVVVSTAHGLKFTQSKIDYHDRNIKDMLCQYANPPINVKADFASVMDVLQNKLNGKI

SEQ ID NO: 25 - DNA, 495 bp, Oryza sativa
gaacacctcactccaatcagcagcaatggaggatcatcagggtggcggtgtaggcagggcgagcaacaagatcagggacatcgtgaggctgcagcagctgctcaagaggtggaagaagctggcgaccatggcgccgggggggaggagcggcgtgcccaaggggtcgttcgcggtgtacgtcggcgaggagatgcggcggttcgtgatcccgacggagtacctcggccactgggcgttcgagcggctgctccgcgacgccgaggaggagttcggcttccgccaccagggcgccctccggatcccctgcgacgtcgccgccttcgaggccaccctccgcctcgtcgccgccggcaacggcaacgccaaggccaaggacgacgccgccgccatgtgctcctgctcctccgacaccgagatcttgtgcagatgatgatgatcaacaccatttcgccatttgtgtgtgcgtgtgtgttttcctctctctcctttcttgcg

SEQ ID NO: 26 - DNA, 405 bp, Oryza sativa
atggaggatcatcagggtggcggtgtaggcagggcgagcaacaagatcagggacatcgtgaggctgcagcagctgctcaagaggtggaagaagctggcgaccatggcgccgggggggaggagcggcgtgcccaaggggtcgttcgcggtgtacgtcggcgaggagatgcggcggttcgtgatcccgacggagtacctcggccactgggcgttcgagcggctgctccgcgacgccgaggaggagttcggcttccgccaccagggcgccctccggatcccctgcgacgtcgccgccttcgaggccaccctccgcctcgtcgccgccggcaacggcaacgccaaggccaaggacgacgccgccgccatgtgctcctgctcctccgacaccgagatcttgtgcagatga

SEQ ID NO: 27 - protein, 134 aa, Oryza sativa
MEDHQGGGVGRASNKIRDIVRLQQLLKRWKKLATMAPGGRSGVPKGSFAVYVGEEMRRFVIPTEYLGHWAFERLLRDAEEEFGFRHQGALRIPCDVAAFEATLRLVAAGNGNAKAKDDAAAMCSCSSDTEILCR

SEQ ID NO: 28 - DNA, 921 bp, Oryza sativa
ggagagagagagagagagaatgggcgaccgggcgtacgtgccggcatcgaagccggtgccggtggcggcggcgcgggcggcgaacggggtggcgaacggaggcggaggaggggttgggggtgggggagggggaggggcggcgcggccgccgcccatggtgccagggcgcgtgcccccgccgccgatgtacaggccgaagccgatgcaagcgccggcgaggcggaggcggagccggcgcgggtggtgctgcgcgtgctgcctgtggatgacgctggtggtggtggggctggtgttcctgggcgccatcgcggcgggggtgttctacgtggcgtaccacccgcagctccccaccttcgccgtcacgtccctccgcctcgccgcgctcaacgtgtccgactccgacgccgtcacctcccgcatcgagttcaccgtcaccgcccgcaaccccaacgacaagatcgccttcgcgtacggcgacatcgcggccgcgttcgccgcggacggcgccgacgtcggcgacggcacggtcccggggttcgtccaccccgccggcaacaccaccgtcatcaagggcgacgcctccgccgccgccgccaccgtggacccgctggtggcgaacggcctcagatccaggaagtcgcacgccatgtcggtggagatggactccaaggttgggttccagatcggccgcttcaagtccaagcgcatcaacgtccgcgtcctctgcgccggcttcaccgccgccctcgccaagaacaccccctccgctccaccgatcgtcgtcgccgccgccccgtcgccggtgaggtcggtcgtcaaggcctcctcctcctcctcgagcacgacggacgccaagtgtaagctccgggtcaagatctggatttggacattttgacggatttgacggtagagaagacttcc

SEQ ID NO: 29 - DNA, 876 bp, Oryza sativa
atgggcgaccgggcgtacgtgccggcatcgaagccggtgccggtggcggcggcgcgggcggcgaacggggtggcgaacggaggcggaggaggggttgggggtgggggagggggaggggcggcgcggccgccgcccatggtgccagggcgcgtgcccccgccgccgatgtacaggccgaagccgatgcaagcgccggcgaggcggaggcggagccggcgcgggtggtgctgcgcgtgctgcctgtggatgacgctggtggtggtggggctggtgttcctgggcgccatcgcggcgggggtgttctacgtggcgtaccacccgcagctccccaccttcgccgtcacgtccctccgcctcgccgcgctcaacgtgtccgactccgacgccgtcacctcccgcatcgagttcaccgtcaccgcccgcaaccccaacgacaagatcgccttcgcgtacggcgacatcgcggccgcgttcgccgcggacggcgccgacgtcggcgacggcacggtcccggggttcgtccaccccgccggcaacaccaccgtcatcaagggcgacgcctccgccgccgccgccaccgtggacccgctggtggcgaacggcctcagatccaggaagtcgcacgccatgtcggtggagatggactccaaggttgggttccagatcggccgcttcaagtccaagcgcatcaacgtccgcgtcctctgcgccggcttcaccgccgccctcgccaagaacaccccctccgctccaccgatcgtcgtcgccgccgccccgtcgccggtgaggtcggtcgtcaaggcctcctcctcctcctcgagcacgacggacgccaagtgtaagctccgggtcaagatctggatttggacattttga

SEQ ID NO: 30 - protein, 291 aa, Oryza sativa
MGDRAYVPASKPVPVAAARAANGVANGGGGGVGGGGGGGAARPPPMVPGRVPPPPMYRPKPMQAPARRRRSRRGWCCACCLWMTLVVVGLVFLGAIAAGVFYVAYHPQLPTFAVTSLRLAALNVSDSDAVTSRIEFTVTARNPNDKIAFAYGDIAAAFAADGADVGDGTVPGFVHPAGNTTVIKGDASAAAATVDPLVANGLRSRKSHAMSVEMDSKVGFQIGRFKSKRINVRVLCAGFTAALAKNTPSAPPIVVAAAPSPVRSVVKASSSSSSTTDAKCKLRVKIWIWTF

SEQ ID NO: 31 - DNA, 30 bp, Artificial Sequence
  Forward primer for cloning gDNA of  OsDN-DRT20 gene
gagctgctgtcttgtttgtttggctggatc

SEQ ID NO: 32 - DNA, 25 bp, Artificial Sequence
  Reverse primer for cloning gDNA of  OsDN-DRT20 gene
gaatgctcaatcgatcagacatgtg

SEQ ID NO: 33 - DNA, 31 bp, Artificial Sequence
  Forward primer for cloning cDNA of  OsEIN3-1 gene
ctgctgaggatgggaggtggtctggtgatgg

SEQ ID NO: 34 - DNA, 33 bp, Artificial Sequence
  Reverse primer for cloning cDNA of  OsEIN3-1 gene
ccgctgaggtcagtagtaccaattcgagccgtc

SEQ ID NO: 35 - DNA, 36 bp, Artificial Sequence
  Forward primer for cloning cDNA of OsCYP-1 gene
ctgctgagggacaaagataagtgaagtgagcaggcg

SEQ ID NO: 36 - DNA, 33 bp, Artificial Sequence
  Reverse primer for cloning cDNA of OsCYP-1 gene
ccgctgaggctgttcggttttcactcctgctcg

SEQ ID NO: 37 - DNA, 35 bp, Artificial Sequence
  Forward primer for cloning cDNA of OsNAC67-3 gene
ctgctgagggccacagagagagcagtagtagtagc

SEQ ID NO: 38 - DNA, 32 bp, Artificial Sequence
  Reverse primer for cloning cDNA of OsNAC67-3 gene
ccgctgaggtttggtcgtctagaatggcttgc

SEQ ID NO: 39 - DNA, 36 bp, Artificial Sequence
  Forward primer for cloning gDNA of OsDN-DTP21 gene
ctgctgaggcattgtgaccatccatccatcgatctc

SEQ ID NO: 40 - DNA, 37 bp, Artificial Sequence
  Reverse primer for cloning gDNA of OsDN-DTP21 gene
ccgctgagggctaggtgtgggagttgtagaggtggag

SEQ ID NO: 41 - DNA, 32 bp, Artificial Sequence
  Forward primer for cloning cDNA of OsSIP1 gene
ctgctgagggagtggagcgatctcgatggacc

SEQ ID NO: 42 - DNA, 32 bp, Artificial Sequence
  Reverse primer for cloning cDNA of OsSIP1 gene
ccgctgaggcgctagctgtacaatgttgttcc

SEQ ID NO: 43 - DNA, 25 bp, Artificial Sequence
  Forward primer for cloning gDNA of OsDC1D1 gene
gggtgaaaatatctggagaacaagc

SEQ ID NO: 44 - DNA, 24 bp, Artificial Sequence
  Reverse primer for cloning gDNA of OsDC1D1 gene
cgagcacatatatacggccttaag

SEQ ID NO: 45 - DNA, 24 bp, Artificial Sequence
  Forward primer for cloning cDNA of OsTNS1 gene
ccatcctcctccctaattactccc

SEQ ID NO: 46 - DNA, 27 bp, Artificial Sequence
  Reverse primer for cloning cDNA of OsTNS1 gene
gctcagatcttaccattcagcttgttc

SEQ ID NO: 47 - DNA, 33 bp, Artificial Sequence
  Forward primer for cloning cDNA of OsSAUR27 gene
ctgctgagggaacacctcactccaatcagcagc

SEQ ID NO: 48 - DNA, 36 bp, Artificial Sequence
  Reverse primer for cloning cDNA of OsSAUR27 gene
ccgctgaggcgcaagaaaggagagagaggaaaacac

SEQ ID NO: 49 - DNA, 28 bp, Artificial Sequence
  Forward primer for cloning cDNA of OsHIP1 gene
ggagagagagagagagagaatgggcgac

SEQ ID NO: 50 - DNA, 26 bp, Artificial Sequence
  Reverse primer for cloning cDNA of OsHIP1 gene
ggaagtcttctctaccgtcaaatccg

SEQ ID NO: 51 - DNA, 21 bp, Artificial Sequence
  Forward primer for real-time PCR analysis of OsDN-DRT20 gene
gatgaaccctatggatcgctc

SEQ ID NO: 52 - DNA, 23 bp, Artificial Sequence
  Reverse primer for real-time PCR analysis of OsDN-DRT20 gene
ccgaatcaggtggagatttatgg

SEQ ID NO: 53 - DNA, 19 bp, Artificial Sequence
  Forward primer for real-time PCR analysis of OsNAC67-3 gene
gctgtgccggatttacaac

SEQ ID NO: 54 - DNA, 18 bp, Artificial Sequence
  Reverse primer for real-time PCR analysis of OsNAC67-3 gene
caccatcggcttcctctg

SEQ ID NO: 55 - DNA, 18 bp, Artificial Sequence
  Forward primer for real-time PCR analysis of OsSIP1 gene
cgagctcgaaccggtacc

SEQ ID NO: 56 - DNA, 19 bp, Artificial Sequence
  Reverse primer for real-time PCR analysis of OsSIP1 gene
cggacagggtgagcaaatc

SEQ ID NO: 57 - DNA, 21 bp, Artificial Sequence
  Forward primer for real-time PCR analysis of OsTNS1 gene
aggaacatcaaggacatgctg

SEQ ID NO: 58 - DNA, 21 bp, Artificial Sequence
  Reverse primer for real-time PCR analysis of OsTNS1 gene
catcacagaagcaaagtcagc

SEQ ID NO: 59 - DNA, 19 bp, Artificial Sequence
  Forward primer for real-time PCR analysis of OsHIP1 gene
aacaccaccgtcatcaagg

SEQ ID NO: 60 - DNA, 20 bp, Artificial Sequence
  Reverse primer for real-time PCR analysis of OsHIP1 gene
cgatctggaacccaaccttg

SEQ ID NO: 61 - DNA, 1569 bp, Oryza sativa; identical to SEQ ID NO: 2

SEQ ID NO: 62 - protein, 522 aa, Oryza sativa; identical to SEQ ID NO: 3

SEQ ID NO: 63 - DNA, 1428 bp, Zea mays
atgcttcgctgtgccgtcgggctcgcaagcattctctttcaggttccaggctctgggtcgccggttgtgccgactccgcccccgcgactgctgcgagcatcgcgagttcgagacgagtttccttcaagcatagagatcagcgcgatggatgacttctctatgatcagtgaccacagcatgctactccaaggccacggcatgctcggtgccgaaggatgccttggtacccgaggttcttcaggcgttcttccgactggaggccgcaggaccgtcccagtcccagcgctccaactccaagatgaccgcaacaacaagggggaagacatgttcttctccgactggcctgagctggccagcttcgacgatctcgaggctagcctgagaaatttcgatcctacgtttgagattgggagcagttattttgatgacatgctatggccctcaaattgctcaccgggagctcagctagcacggaacggctactctgacgatattgatttctcaatcgatcaaaaagacagcaacagcactccgaaggtaaatacgacaaaaaccaagcaacagtccaggagaaacggagctagtggtggtagcagtggtacagcctcgaaccacgaagcacatgccagctcctcttccggtctttcggacgccgaactcttcctccacccgttcgatgatactacagcactagcgagccaaacgtgggaggaggacctacaggccattctttgttcgattccggaaacgcgagcagtcgtcccagcggcgtcaaccgccatgtgcgccgatggctcgtccacctgttgctcggggccagacatcgttgccgctcaccacgttcctcgctcggcgacggcgacgactaaagacgccgcctttagtgggtctccggacacgatcctggaggagatggctgagaacccgctggacatgtacttccctccactgccaacaacatccggacagtccggaacgatgatgatgagcgacaccacttgggcgccggaacatcggttccgagaagagttcgcgggcagctgcgctctggggtgcgcggagctacggttctgctcggaggacgtggcctccgcaggagtatttcataagcagcctggctcggcgacggcggtcgtcctggatgccgtgccagtgaaggatctttcgtttcagaagcttcagcatggcatgaaccagcttgatctggccaccagaggacgcatacgggattccctgtaccggttggccaacaggcttgaacaaacgcattgcgttgccagaacaagcggaagaatgggttcaaataggttcgaatcggacgggtggggcgaaacgcagacgacgagccccatggatcggttagtcgcgcagctccttctgcagaaaccctctcgccggaagactaccccgccgcaccgcgtgacgtag

SEQ ID NO: 64 - protein, 475 aa, Zea mays
MLRCAVGLASILFQVPGSGSPVVPTPPPRLLRASRVRDEFPSSIEISAMDDFSMISDHSMLLQGHGMLGAEGCLGTRGSSGVLPTGGRRTVPVPALQLQDDRNNKGEDMFFSDWPELASFDDLEASLRNFDPTFEIGSSYFDDMLWPSNCSPGAQLARNGYSDDIDFSIDQKDSNSTPKVNTTKTKQQSRRNGASGGSSGTASNHEAHASSSSGLSDAELFLHPFDDTTALASQTWEEDLQAILCSIPETRAVVPAASTAMCADGSSTCCSGPDIVAAHHVPRSATATTKDAAFSGSPDTILEEMAENPLDMYFPPLPTTSGQSGTMMMSDTTWAPEHRFREEFAGSCALGCAELRFCSEDVASAGVFHKQPGSATAVVLDAVPVKDLSFQKLQHGMNQLDLATRGRIRDSLYRLANRLEQTHCVARTSGRMGSNRFESDGWGETQTTSPMDRLVAQLLLQKPSRRKTTPPHRVT

SEQ ID NO: 65 - DNA, 1254 bp, Sorghum bicolor
atggatgatttttctactctcagtgaccacagtatgctacttcaaggccatgacatgttcagtgctgaaggatgcctaggtatccgcagtcctccaggcgttctttcgaccggaggcaagcccgtcgtcccagaactccaagatgcccacaacagcaagggggatgatatgttcttctccgactggcctgagctggtcgccttcgacgatctcgaggcaagcctgagaaatttcgatccaacgtttgagatagggagcaattatttcgaggacatactatggtcctcaaattgctcaccagaagctcagctagtacggaacagctactctgacgatattgatttctcaatcgatcgaaacgacagcaacactccgaaggtaaatacgacaaaaaccaagcaacagtccagcaggaacggagcaatcagtagtggtacagcctcgaattatgatgcacatgccagctcctcttccggtctttgggatgccgaactcttcctcccgtttgatgatacatcactcgccagccaaacgggtggctgggaagggctagaggctattctttgctcatcgagtgcggaaatgcgagtagtcccagcggcatcaagcaccatgtgcaccgatggttcgtctacttgttgctcagggccagacaccgttactgctcgtgatgctcctggttctgcgacgaaggctagagacccgtttaacggggctccggatacaatcctggaggagatggctgaaaatccactggacatgtattttcctccactggcaacatgtgaacgacagcctgagatgttgaagagcgacaccacttcagcgccgaagcatcggtttccagaagagtttgctgcaggcagctgcgctctggagtgtgcagagttacagttctgtgcggaggacatgagttctgcaggattacatgggcagcctggctcggcaatcgttctggacgccgtgccagtaaaggatctttcctttcagaagcttcagtatggcatgaatcagctcggtctggagaccaaaggacgcataagggattcgctataccggttggccaacaggcttgaacaaaagcatcgtgttgcttgttcaagtgaaggattgggatcatcgagttcagataggttcgaatcaggcagatggaccgagacgcagacgaaccccatggatcagtcagtagcacagctccttctgcagaaaccctcttaccggaagactgtcccgccgccgcaccgtgtgacatag

SEQ ID NO: 66 - protein, 417 aa, Sorghum bicolor
MDDFSTLSDHSMLLQGHDMFSAEGCLGIRSPPGVLSTGGKPVVPELQDAHNSKGDDMFFSDWPELVAFDDLEASLRNFDPTFEIGSNYFEDILWSSNCSPEAQLVRNSYSDDIDFSIDRNDSNTPKVNTTKTKQQSSRNGAISSGTASNYDAHASSSSGLWDAELFLPFDDTSLASQTGGWEGLEAILCSSSAEMRVVPAASSTMCTDGSSTCCSGPDTVTARDAPGSATKARDPFNGAPDTILEEMAENPLDMYFPPLATCERQPEMLKSDTTSAPKHRFPEEFAAGSCALECAELQFCAEDMSSAGLHGQPGSAIVLDAVPVKDLSFQKLQYGMNQLGLETKGRIRDSLYRLANRLEQKHRVACSSEGLGSSSSDRFESGRWTETQTNPMDQSVAQLLLQKPSYRKTVPPPHRVT

SEQ ID NO: 67 - DNA, 1455 bp, Glycine max
atgtctgatcattgtttgaagagcagcaaagtagattctagtagtagtgagctttgtgcagatgataccatcttaggagacaagtgtgtggtggaggatgatagcgtgtctcaatattcaatcaatcacatatctcaaactgacaatgaactcagctttcttgataatgatgggtggctcaatatcggaaactttgaagatgttgataggatgatgttaagctgtgacttgacatttggaatggagagcctcaataatgaagaggagttctgctggctccggtcttcaaatggaactgaaggatctgatgatgcattgaagtctgcattcaagttttcatctgctgaagcaagtctgttgaaaagcatatcagattataatatcgacacaaatgaaaacatggaagccatgaaatctggtaatagagatgacttgatggctaaactaaagatggaaggaaacctgttaaaaacatcagcaggaaagagaaaaaatggttacctgggacatggtgattttgatcttccttatgctcaagtggagcaatatgcaaatctgaagcaatcttttggagcctcttccagtggggtcacttcacaggatagcatccacaaacacagaccagacatggattctaattctttaggacatatacagatacaaactgacctaatggacccaggctattgtcatacttctaattacacttccctccttccaactttgtctggatctaggtctggacatgatggacatccatctccttcttttaaagaatcgtcatttgcacctaacatggagagttctaatgctcataagttggatgctgttgccttgaaaacaaaaaatgagagagaaaatttatatttttgccacgatgcacaactaataactcagaaggtaggccatcagtttgaaaatgagaatgaaggccatagtgaagttggagaaattagcataagattttcacaagaaatagactcatcaaatgtgcaggaaagctcatctatgagctctgcactggacgaagcctcacttgaaacaactagcttttgccaactgcaacagatcatggatcagttggatattagaaccaaactatgcataagggacagtctataccgcttggctaaaagtgctgaacaaagacataatgataccaatgcaagtggtcaaattggtgatgatgttgaagcctgcaaagcagtaatgatacaggactcaaacaggtgtacgggattcatgcatattgaaactgatacaaatcccgttgatcgaactgttgcacacttactgtttcacaggccttcagatccatcaatgttgccccataatgatcctttacctttcaagtccagttccatgttatgtggatcggtgatcaatccagcagtagtgactgagaaagaggtttgtcaggaagaatcttctactggattagaaaagtcgtcttaa

SEQ ID NO: 68 - protein, 484 aa, Glycine max
MSDHCLKSSKVDSSSSELCADDTILGDKCVVEDDSVSQYSINHISQTDNELSFLDNDGWLNIGNFEDVDRMMLSCDLTFGMESLNNEEEFCWLRSSNGTEGSDDALKSAFKFSSAEASLLKSISDYNIDTNENMEAMKSGNRDDLMAKLKMEGNLLKTSAGKRKNGYLGHGDFDLPYAQVEQYANLKQSFGASSSGVTSQDSIHKHRPDMDSNSLGHIQIQTDLMDPGYCHTSNYTSLLPTLSGSRSGHDGHPSPSFKESSFAPNMESSNAHKLDAVALKTKNERENLYFCHDAQLITQKVGHQFENENEGHSEVGEISIRFSQEIDSSNVQESSSMSSALDEASLETTSFCQLQQIMDQLDIRTKLCIRDSLYRLAKSAEQRHNDTNASGQIGDDVEACKAVMIQDSNRCTGFMHIETDTNPVDRTVAHLLFHRPSDPSMLPHNDPLPFKSSSMLCGSVINPAVVTEKEVCQEESSTGLEKSS

SEQ ID NO: 69 - DNA, 1932 bp, Oryza sativa
atgggaggtggtctggtgatggaccagggcatgatgttccccggcgtgcacaacttcgtggatctcctgcagcagaacggcggcgacaagaacctcggcttcggcgcgctcgtgccgcagacgtcgtcgggggagcagtgcgtgatgggggagggcgacctcgtggacccgccgccggagagcttcccggacgccggtgaggacgacagcgacgacgacgtggaggacatcgaggagctggagcgccgcatgtggcgcgaccgcatgaagctgaagcggctcaaggagctgcagctgagccggggcaaggaccccgcgggcggcgtcgtgggcgacccgtccaagccgcggcagtcgcaggagcaggcgcggcggaagaagatgtcgcgcgcgcaggacggcatcctcaagtacatgctcaagatgatggaggtgtgccgcgcgcaggggttcgtgtacgggatcatcccggagaagggcaagccggtgagcggcgcctccgacaacctccgcggctggtggaaggagaaggtccgcttcgaccgcaacggccccgccgccatcgccaagtaccaggccgacaacgccgtcccgggcttcgagagcgagctcgcctccggcaccgggagcccgcactcgctgcaggagctgcaggacaccaccctcgggtcgctgctctcggcgctcatgcagcactgcgaccctccgcagcggcggtacccgctcgagaagggcgtccctccgccgtggtggcccaccggcgacgaggagtggtggccggagctcggcatccccaaggaccagggcccgcctccgtacaagaagccccatgacctcaagaaggcctggaaggtcagcgtgctcaccgctgtcatcaagcacatgtcgccggacatcgagaagatccgccggctggtccggcagtccaagtgcctccaggacaagatgaccgccaaggagatctccacctggctggccgtcgtcaagcaggaagaggagctgtacctgaagctgaaccccggtgcccgccctccggcacctaccggcggcatcaccagcgccatatcgttcaacgccagctcaagtgagtacgacgtcgacgtcgtcgacgactgcaagggcgacgaggccggcaaccagaaggctgttgttgtcgccgacccgaccgcgttcaacctcggcgcggctatgctgaacgacaagttcctcatgccggcgtccatgaaggaggaggccaccgatgtcgagttcatccagaagaggagcgcgtctggcgcggagcctgagctgatgctgaacaaccgtgtctacacctgccacaatgtccagtgcccgcatagcgactatggatacgggttccttgaccggaacgcgcgcaacagccaccaatacacttgcaagtacaatgatccactccagcagagcacggagaacaagccatcgccaccggccatcttcccggcaacctacaacacgccgaaccaggctctgaacaatctggatttcggcctgcccatggatggccagaggtcaattacagagctgatgaacatgtacgacaacaacttcgtggccaacaagaaccttagcaacgacaatgccacgatcatggagaggcctaatgcagtcaacccaaggatacagattgaagaaggcttttttggacagggaagtggcatcggcggcagcaacggaggtgtgttcgaagatgtcaatggcatgatgcagcaaccgcagcagaccaccccggcacagcagcagttcttcatccgcgacgatactccattcggtaaccagatgggcgacatcaatggcgcatcggagttcaggttcggctctggtttcaacatgtcaggtgccgtcgaataccccggcgcaatgcagggccagcagaagaatgacggcgcatcggagtttgaggaattggaatga

SEQ ID NO: 70 - protein, 643 aa, Oryza sativa
MGGGLVMDQGMMFPGVHNFVDLLQQNGGDKNLGFGALVPQTSSGEQCVMGEGDLVDPPPESFPDAGEDDSDDDVEDIEELERRMWRDRMKLKRLKELQLSRGKDPAGGVVGDPSKPRQSQEQARRKKMSRAQDGILKYMLKMMEVCRAQGFVYGIIPEKGKPVSGASDNLRGWWKEKVRFDRNGPAAIAKYQADNAVPGFESELASGTGSPHSLQELQDTTLGSLLSALMQHCDPPQRRYPLEKGVPPPWWPTGDEEWWPELGIPKDQGPPPYKKPHDLKKAWKVSVLTAVIKHMSPDIEKIRRLVRQSKCLQDKMTAKEISTWLAVVKQEEELYLKLNPGARPPAPTGGITSAISFNASSSEYDVDVVDDCKGDEAGNQKAVVVADPTAFNLGAAMLNDKFLMPASMKEEATDVEFIQKRSASGAEPELMLNNRVYTCHNVQCPHSDYGYGFLDRNARNSHQYTCKYNDPLQQSTENKPSPPAIFPATYNTPNQALNNLDFGLPMDGQRSITELMNMYDNNFVANKNLSNDNATIMERPNAVNPRIQIEEGFFGQGSGIGGSNGGVFEDVNGMMQQPQQTTPAQQQFFIRDDTPFGNQMGDINGASEFRFGSGFNMSGAVEYPGAMQGQQKNDGASEFEELE

SEQ ID NO: 71 - DNA, 1929 bp, Zea mays
atgatgggaggcgggctgttggtggatcagagcgtggtgttccctggcgtccacaacttcgtggatctcctgcagcagaacggcgacaagaacctgggcttcgggtctctgatgccgcagacgtcctctggcgaccagtgcgtgatgggggagggcgatctcgtggacccgcctccggatagcttcccggacgccggggaggacgacagcgacgatgacgtcgaggacatcgaggagctggagcgccgcatgtggcgcgaccgtatgaagctgaagcggctcagggaactgcagcagacccgcggcaaggactcgttggctagcggtgcgggactggctgatggctcgtccaagccaaggcagtcgcaggagcaggcccggcgcaagaagatgtcgcgcgcgcaggacggcatcctcaagtacatgctcaagatgatggaggtgtgccgcgcgcaggggtttgtgtatgggatcattccggagaagggcaagccagtgagtggtgcctccgacaacctccgtgcgtggtggaaggagaaggtccgcttcgaccgcaacggaccggccgccattgccaagtaccaggccgacaacgccgtccctggcgccgagaacgagctcgcctcgggcgctgccagcccccattccttgcaggagctgcaggacactacgctgggctcactgctctcagcgcttatgcagcactgcgagcccccacagcggcgctacccgctcgagaagggcgttcctccaccgtggtggcctaccggcgacgaggagtggtggccggaactcggcattcccaaggaccagggcccacccccgtacaagaagcctcatgaccttaagaaggcctggaaggtgagcgtgctcaccgctgtcatcaagcacatgtcaccggacatagagaagatccgtcgcctggttcgccagtccaagtgcctccaggacaagatgactgccaaggagatctcaacctggctggcggtcgtcaagcaggaagaggagctgtaccagaagctgaacccgggcgcacgcccaccggcgtctactggtggcatcgcaagtgccatatccttcaacaccagctcgagcgagtatgatgtggacatcatcgatgagtgcaagggggacgaggccggtaaccagaggacggcagtcactgacccaaccgcgttcaaccttggtgccgctatcctaagcgacaagttcctcgtgccgacgccgatgaaggaggagaccgccgacgtggagttcatccagaagaggaacgcccccgctgcagccgagccagagctgatgctaaacaaccgattgtacacctgcaacaacgtccagtgcccgcgcagtgactacagctacggattcctggaccggaatgcccgcaacagccaccagtacacctgcaagcacaaggatccaacccctcagagcaccgagaacaagccgccgtcagcaccgccacagccacaagccttccagccggccttcagccaacccaaccaggcactgaacagtctggatttcagcctgcccatggacgggcagaggtccatcgccgagctgatgaacatgtacgacaacaacttcgtgccgaacaagaacccgagcagcgacagcgtcgccgtcatggagaggccaaacgcgatgccccagcagaggatccagatggacgagggtttcttcgtacagggcaacggagccttcgacgacgtcaacaacagcatgatgcagcagcagcagcagcaggcgccagtgcagcagcagcagcagttcttcatccgcgacgacacgccattcgtgagccagatgggcgacatcgccgccagcgcgccggagttcaggttcggtcctggtttcaacatgtctagcggcgtcgactacccaggcgcggcacagaggaacgacgggaccaattggttctactga

SEQ ID NO: 72 - protein, 642 aa, Zea mays
MMGGGLLVDQSVVFPGVHNFVDLLQQNGDKNLGFGSLMPQTSSGDQCVMGEGDLVDPPPDSFPDAGEDDSDDDVEDIEELERRMWRDRMKLKRLRELQQTRGKDSLASGAGLADGSSKPRQSQEQARRKKMSRAQDGILKYMLKMMEVCRAQGFVYGIIPEKGKPVSGASDNLRAWWKEKVRFDRNGPAAIAKYQADNAVPGAENELASGAASPHSLQELQDTTLGSLLSALMQHCEPPQRRYPLEKGVPPPWWPTGDEEWWPELGIPKDQGPPPYKKPHDLKKAWKVSVLTAVIKHMSPDIEKIRRLVRQSKCLQDKMTAKEISTWLAVVKQEEELYQKLNPGARPPASTGGIASAISFNTSSSEYDVDIIDECKGDEAGNQRTAVTDPTAFNLGAAILSDKFLVPTPMKEETADVEFIQKRNAPAAAEPELMLNNRLYTCNNVQCPRSDYSYGFLDRNARNSHQYTCKHKDPTPQSTENKPPSAPPQPQAFQPAFSQPNQALNSLDFSLPMDGQRSIAELMNMYDNNFVPNKNPSSDSVAVMERPNAMPQQRIQMDEGFFVQGNGAFDDVNNSMMQQQQQQAPVQQQQQFFIRDDTPFVSQMGDIAASAPEFRFGPGFNMSSGVDYPGAAQRNDGTNWFY

SEQ ID NO: 73 - DNA, 1932 bp, Sorghum bicolor
atgatgggaggcgggctgatgatggatcagagcgtggtgttccctggcgtccacaacttcgtggatctccttcagcaaaacggtgacaagaaccttggcttcgggtcgctgatgccgcagacgtcctccggcgaccagtgcgtgatgggggagggtgatcttgtggacccgccgccggagagcttcccggacgctggggaggacgacagcgatgatgatgttgaggacatcgaggagctggagcgccgcatgtggcgcgaccgcatgaagctgaagcggctcagggagctgcagcagagccgcggcaaggattcgatagctggcggtgggggcctggctgacggctcgtccaagccaaggcagtcgcaggagcaggcccgacgcaagaagatgtctcgcgcgcaggacggcatcctcaagtacatgctcaagatgatggaagtgtgccgcgcacaggggtttgtgtatgggatcattccggagaagggcaagccggtgagcggcgcctccgacaacctccgtgcgtggtggaaggagaaggtccgcttcgaccgcaacggcccggccgccatcgccaagtatcaggccgacaacgcagtccctggtgccgagaacgagctcacctcgggcgctgccagccctcattccttgcaggagctgcaggacactacgctgggctcattgctctcagcactcatgcagcactgcgaccccccacagcggcgctacccgctggagaagggcgttcctccaccatggtggcctactggcgacgaagagtggtggccggaacttggcatccccaaggaccagggcccacccccatacaagaagcctcatgaccttaagaaggcctggaaggtgagcgtgctcaccgctgtcatcaagcacatgtcaccagacatagagaagatccgtcgccttgttcgccagtccaagtgcctccaggacaagatgactgccaaggagatctcaacctggctggcggtcgtcaagcaggaagaggagctgtacctgaagctgcaccccggcgcactcccaccagcatctactggtggcatcgccagtgccatatccttcaacaccagctcaagcgagtatgatgtggacatcattgatgagtgtaagggggatgaggccggcaaccagaagacaggagtcactgacccaaccgcgttcaaccttggtgctgctatcctaagtgacaagttccttgtgcagacgcccatgaaggaggagactgcggacgtcgagttcatccagaagaggaacgcccccgctgctgctgagccagagctaatgctaaacaaccgagtgtacacctgcaacaacgtccagtgcccacacagtgactacagctatggattccttgaccggaatacccgcaacagccaccagtacacctgtaagtacaatgaaccaatccctcagagcactgagaacaagccgccgccagcaccgccacagtcacaagccttccagccggccttcaaccaacccaatcagtcactgaacaatctggatttcagcctgcccatggacgggcagaggtccatcgctgagctgatgaacatgtacgacaacaacttcatgacaaacaagaacatgagcagtgacagcgtcaccatcatggagaggcctaatgcgatgccccagaggatccagatggatgagggtttctttggacagggcaatggagtcttcgacgatgtcaatagcatgatgcagcaacaacagcaggcaccagtgcagcagcagcagcagcagcagcagcagcagttcttcatccgtgatgacacgccatttgtgagccagatgggcgacatcaccagcacatcggagttcaggttcggttctggtttcaacatgtctagcaccgttgattacccaggcgcggcgcagaagaacgatgggaccaattggttctactga

SEQ ID NO: 74 - protein, 643 aa, Sorghum bicolor
MMGGGLMMDQSVVFPGVHNFVDLLQQNGDKNLGFGSLMPQTSSGDQCVMGEGDLVDPPPESFPDAGEDDSDDDVEDIEELERRMWRDRMKLKRLRELQQSRGKDSIAGGGGLADGSSKPRQSQEQARRKKMSRAQDGILKYMLKMMEVCRAQGFVYGIIPEKGKPVSGASDNLRAWWKEKVRFDRNGPAAIAKYQADNAVPGAENELTSGAASPHSLQELQDTTLGSLLSALMQHCDPPQRRYPLEKGVPPPWWPTGDEEWWPELGIPKDQGPPPYKKPHDLKKAWKVSVLTAVIKHMSPDIEKIRRLVRQSKCLQDKMTAKEISTWLAVVKQEEELYLKLHPGALPPASTGGIASAISFNTSSSEYDVDIIDECKGDEAGNQKTGVTDPTAFNLGAAILSDKFLVQTPMKEETADVEFIQKRNAPAAAEPELMLNNRVYTCNNVQCPHSDYSYGFLDRNTRNSHQYTCKYNEPIPQSTENKPPPAPPQSQAFQPAFNQPNQSLNNLDFSLPMDGQRSIAELMNMYDNNFMTNKNMSSDSVTIMERPNAMPQRIQMDEGFFGQGNGVFDDVNSMMQQQQQAPVQQQQQQQQQQFFIRDDTPFVSQMGDITSTSEFRFGSGFNMSSTVDYPGAAQKNDGTNWFY

SEQ ID NO: 75 - DNA, 1887 bp, Arabidopsis thaliana
atgatgtttaatgagatgggaatgtgtggaaacatggatttcttctcttctggatcacttggtgaagttgatttctgtcctgttccacaagctgagcctgattccattgttgaagatgactatactgatgatgagattgatgttgatgaattggagaggaggatgtggagagacaaaatgcggcttaaacgtctcaaggagcaggataagggtaaagaaggtgttgatgctgctaaacagaggcagtctcaagagcaagctaggaggaagaaaatgtctagagctcaagatgggatcttgaagtatatgttgaagatgatggaagtttgtaaagctcaaggctttgtttatgggattattccggagaatgggaagcctgtgactggtgcttctgataatttaagggagtggtggaaagataaggttaggtttgatcgtaatggtcctgcggctattaccaagtatcaagcggagaataatatcccggggattcatgaaggtaataacccgattggaccgactcctcataccttgcaagagcttcaagacacgactcttggatcgcttttgtctgcgttgatgcaacactgtgatcctcctcagagacgttttcctttggagaaaggagttcctcctccgtggtggcctaatgggaaagaggattggtggcctcaacttggtttgcctaaagatcaaggtcctgcaccttacaagaagcctcatgatttgaagaaggcgtggaaagtcggcgttttgactgcggttatcaagcatatgtttcctgatattgctaagatccgtaagctcgtgaggcaatctaaatgtttgcaggataagatgactgctaaagagagtgctacctggcttgctattattaaccaagaagagtccttggctagagagctttatcccgagtcatgtccacctctttctctgtctggtggaagttgctcgcttctgatgaatgattgcagtcaatacgatgttgaaggtttcgagaaggagtctcactatgaagtggaagagctcaagccagaaaaagttatgaattcttcaaactttgggatggttgctaaaatgcatgactttcctgtcaaagaagaagtcccagcaggaaactcggaattcatgagaaagagaaagccaaacagagatctgaacactattatggacagaaccgttttcacctgcgagaatcttgggtgtgcgcacagcgaaatcagccggggatttctggataggaattcgagagacaaccatcaactggcatgtccacatcgagacagtcgcttaccgtatggagcagcaccatccaggtttcatgtcaatgaagttaagcctgtagttggatttcctcagccaaggccagtgaactcagtagcccaaccaattgacttaacgggtatagttcctgaagatggacagaagatgatctcagagctcatgtccatgtacgacagaaatgtccagagcaaccaaacctctatggtcatggaaaatcaaagcgtgtcactgcttcaacccacagtccataaccatcaagaacatctccagttcccaggaaacatggtggaaggaagtttctttgaagacttgaacatcccaaacagagcaaacaacaacaacagcagcaacaatcaaacgttttttcaagggaacaacaacaacaacaatgtgtttaagttcgacactgcagatcacaacaactttgaagctgcacataacaacaacaataacagtagcggcaacaggttccagcttgtgtttgattccacaccgttcgacatggcgtcattcgattacagagatgatatgtcgatgccaggagtagtaggaacgatggatggaatgcagcagaagcagcaagatgtatccatatggttctaa

SEQ ID NO: 76 - protein, 628 aa, Arabidopsis thaliana
MMFNEMGMCGNMDFFSSGSLGEVDFCPVPQAEPDSIVEDDYTDDEIDVDELERRMWRDKMRLKRLKEQDKGKEGVDAAKQRQSQEQARRKKMSRAQDGILKYMLKMMEVCKAQGFVYGIIPENGKPVTGASDNLREWWKDKVRFDRNGPAAITKYQAENNIPGIHEGNNPIGPTPHTLQELQDTTLGSLLSALMQHCDPPQRRFPLEKGVPPPWWPNGKEDWWPQLGLPKDQGPAPYKKPHDLKKAWKVGVLTAVIKHMFPDIAKIRKLVRQSKCLQDKMTAKESATWLAIINQEESLARELYPESCPPLSLSGGSCSLLMNDCSQYDVEGFEKESHYEVEELKPEKVMNSSNFGMVAKMHDFPVKEEVPAGNSEFMRKRKPNRDLNTIMDRTVFTCENLGCAHSEISRGFLDRNSRDNHQLACPHRDSRLPYGAAPSRFHVNEVKPVVGFPQPRPVNSVAQPIDLTGIVPEDGQKMISELMSMYDRNVQSNQTSMVMENQSVSLLQPTVHNHQEHLQFPGNMVEGSFFEDLNIPNRANNNNSSNNQTFFQGNNNNNNVFKFDTADHNNFEAAHNNNNNSSGNRFQLVFDSTPFDMASFDYRDDMSMPGVVGTMDGMQQKQQDVSIWF

SEQ ID NO: 77 - DNA, 1833 bp, Glycine max
atgatgatgtttgaagatatgggattctgtggcgatttggatatgttatgtggttctcttggggatggggatattgctgtgagacaaactgaaccggatcctgtagttgaggatgactacagtgatgaagaaattgatgtggatgaactcgagaagaggatgtggagggacaaaatgcgtctcaagcgattgaaagaacaaaccaagtccaaggaagggactgatgcagcaaagcaaaggcaatcccaagagcaggcaaggaggaaaaagatgtcaagagcccaagatggaatactgaagtacatgctgaagatgatggaggtttgcaaggcacaagggtttgtttatgggataattcctgagaaggggaagccagtgaccggagcatcagataatcttcgcgaatggtggaaagataaggtcaggtttgatcgaaatggtcctgctgccatagccaagtatcaagccgataatgcaattcctggaaagaatgatggatgcaattccattggtcctacaccacacaccttgcaagagttacaggacacaaccttgggttctctcttgtcagcacttatgcagcactgtgatcctcctcagaggaggttcccactagagaagggtgttcctccaccatggtggccaactgggaatgaagaatggtggcctcaaattggtctacctaaagatcaaggccctccaccttacaagaaaccacatgacttaaagaaggcgtggaaggttggtgttctcactgcagtcatcaagcatatgtcccctgatatcgccaaaattcgcaagcttgtgaggcagtccaaatgccttcaagacaaaatgacagcaaaggaaagtgcaacctggcttgccatcatcaaccaagaggaagccttggctagagagctttaccctgattattgccctccgttttcctctgctgtagctaatggatccatggtgatcaacgattgcagtgagtatgatgttgatggggctgaagaagagccgaacttcgatgttgaggaccggaagcccgaccatcttcatccatcaaaccttgggatggagagaatgatgggaaggatgccaattcagcaaccttctcatcccatgaagggagatgttgtcacaaacctagatttcatccggaagaggaagatttctagtgacttcaacatgatgatggatcagaaaatctacacatgcgagcatccccaatgcccttacagcgaagttcgccttggtttccatgataggtctgctagggacaatcatcaattgaattgtgcatatagaaacagttctgcagattatggtggtggtcccaatttccatgctactgaggttaagccagtcatattcccccagtcctttgttcaacccaacactacagctcagtctgcaagtttggttgcaccttcatttgatctaactggtcttggagttcctgaggatggccagaaaatgattagtgaccttatgacaatctatgatacaaatgttgtaggaaacaaaaacctaagctccaccaactgtgttactgctgaaaatcataacctttctcaggccagcttacaacgacaggacagttttttccctggtcaaggaatggtgttggaagggaacttgtttgcacgagaggaaggtcaatttgaccggttcaaggccaccatgaacatgaacactccttttgataccaaccacaacaacaataatatccatttgatgtttaattccccttgtgatttgtcatcctttgatttcaaggaggatatacaaggagtaggaatggattctcttcaaaaacagcaagaggtttcaatttggtaccagtga

SEQ ID NO: 78 - protein, 610 aa, Glycine max
MMMFEDMGFCGDLDMLCGSLGDGDIAVRQTEPDPVVEDDYSDEEIDVDELEKRMWRDKMRLKRLKEQTKSKEGTDAAKQRQSQEQARRKKMSRAQDGILKYMLKMMEVCKAQGFVYGIIPEKGKPVTGASDNLREWWKDKVRFDRNGPAAIAKYQADNAIPGKNDGCNSIGPTPHTLQELQDTTLGSLLSALMQHCDPPQRRFPLEKGVPPPWWPTGNEEWWPQIGLPKDQGPPPYKKPHDLKKAWKVGVLTAVIKHMSPDIAKIRKLVRQSKCLQDKMTAKESATWLAIINQEEALARELYPDYCPPFSSAVANGSMVINDCSEYDVDGAEEEPNFDVEDRKPDHLHPSNLGMERMMGRMPIQQPSHPMKGDVVTNLDFIRKRKISSDFNMMMDQKIYTCEHPQCPYSEVRLGFHDRSARDNHQLNCAYRNSSADYGGGPNFHATEVKPVIFPQSFVQPNTTAQSASLVAPSFDLTGLGVPEDGQKMISDLMTIYDTNVVGNKNLSSTNCVTAENHNLSQASLQRQDSFFPGQGMVLEGNLFAREEGQFDRFKATMNMNTPFDTNHNNNNIHLMFNSPCDLSSFDFKEDIQGVGMDSLQKQQEVSIWYQ

SEQ ID NO: 79 - DNA, 1503 bp, Oryza sativa
atggcagcctccttcgtcatcgtcatcgtcatctccttcttcatttctcttgcttttatgtgctatgtccactacacgagccggcagaggaggaaactccatggctacggccatgagaaagccgtcaggctgccgccgggctccatgggttggccttacatcggcgagacccttcagctctactcccaagaccccaacgtcttcttcgcctccaaacagaagaggtacggcgagatcttcaagacgcacattctgggttgcccgtgcgtgatgctggcgagcccggaggcggcgcggttcgtgctggtgacgcaggcgcacctgttcaagccgacgtacccgcggagcaaggagcggatgatcggcccgtcggcgctcttcttccaccagggcgactaccacctccgccttcgcaagctcgtccagggccctctcggccccgacgccctgcgcgcgctcgtgccggacgtcgaggccgccgtccgctccacgctcgcctcctgggacggcaacgtctccagcaccttccacgccatgaagaggctctcgttcgatgtcggcatcgtgaccatcttcggcgggcggctggacgagcggcggaaagcggagctgaggcagaactacgccatcgtggagaagggctacaactccttccccaacagcttccccgggacgctgtactacaaggcgatccaggcgaggcggcggctgcacggcgtgctgagcgacatcatgcgggagcggcgggcgcggggggagcccggcagcgacctcctcggctgcctcatgcagtcgcgggcgggcgacgacggcgcgctcctcaccgacgagcaggtcgccgacaacatcatcggcgtgctgttcgcggcgcaggacacgacggccagcgtgctcacctggatcgtcaagtacctccacgaccatcccaagctgctcgaggccgtcagggcggagcaggcggcgatccgcgccgccaacgacggcggccggctgccgctgacgtgggcgcagacgcggagcatggccctaacccacaaggtgattttggagagcttaaggatggccagcatcatctcgttcacgttcagggaggccgtggctgacgtggagtacaaagggttccttatccccaagggatggaaggtgatgccgctcttcaggaacatccatcacaacccagactacttccaggatccacagaagttcgacccttctagattcaaggtgtcgccgaggccgaacaccttcatgccatttgggaacggcgtgcacgcgtgccccgggaacgagctggccaagctcgagatgctcgtcctcatccaccacctggtcactggctacaggtgggagattgttggttccagcgacgaggttgagtacagcccattccctgtgcccaagcatggccttctcgcgaaattatggagggatgatagtgtcagtgtggaaacagatggttgccagaacggtgataatgacgacaatggcgtagcaatggtttga

SEQ ID NO: 80 - protein, 500 aa, Oryza sativa
MAASFVIVIVISFFISLAFMCYVHYTSRQRRKLHGYGHEKAVRLPPGSMGWPYIGETLQLYSQDPNVFFASKQKRYGEIFKTHILGCPCVMLASPEAARFVLVTQAHLFKPTYPRSKERMIGPSALFFHQGDYHLRLRKLVQGPLGPDALRALVPDVEAAVRSTLASWDGNVSSTFHAMKRLSFDVGIVTIFGGRLDERRKAELRQNYAIVEKGYNSFPNSFPGTLYYKAIQARRRLHGVLSDIMRERRARGEPGSDLLGCLMQSRAGDDGALLTDEQVADNIIGVLFAAQDTTASVLTWIVKYLHDHPKLLEAVRAEQAAIRAANDGGRLPLTWAQTRSMALTHKVILESLRMASIISFTFREAVADVEYKGFLIPKGWKVMPLFRNIHHNPDYFQDPQKFDPSRFKVSPRPNTFMPFGNGVHACPGNELAKLEMLVLIHHLVTGYRWEIVGSSDEVEYSPFPVPKHGLLAKLWRDDSVSVETDGCQNGDNDDNGVAMV

SEQ ID NO: 81 - DNA, 1587 bp, Zea mays
atgggcgcctttctgctcttcgtctgtctcctggcgccggtcgtcgtgctcgcctgcgccgtccgcggcaggaagcggcgggcgtcctccgcggcggcgggcggcaaggcgctgccgctgccgcccgggtcgatggggtggccgtacgtgggcgagacgttccagctctactcgtccaagaaccccaacgtgttcttcgcccggaagcagaaccggtacgggccaatcttcaagacgcacatcctgggctgcccctgcgtgatggtgtccagcccggaggcggcgcgcttcgtgctcgtcacgcaggcccacctcttcaagcccaccttcccggcgagcaaggagcgcatgctggggccgcaggccatcttcttccagcagggcgactaccacgcccacctccgccgcctcgtctcccgggctttctcccccgaggccatccgcggctccgtgccggccatcgaggccatcgcgctgcgctcgctcgagtcctgggacggccgcctcgtcaacaccttccaagagatgaagctgtacgcgctgaatgtggcattgctgtccatcttcggcgaggaagagatgcggtacatagaggagctgaagcagtgctacctgaccctggagaaggggtacaactcgatgcccgtgaacctgccgggcaccctgttccacaaggccatgaaggcccgcaagcgcctgggcgccgtcgtggcccacatcatcgaggcccggcgcgagggcgagcggcagcgcgggagcgacctcctggcctccttcctggacgaccgcgaggcgctcaccgacgcccagatcgccgacaacgtgatcggcgtcatcttcgccgcccgcgacaccaccgccagcgtgctcacctggatggtcaagttcctcggcgaccaccccgccgtcctcaaggccgtcatcgtaagttcttctctccctgccggctcatgcctggcaccagatcgactgcgtgcgcccgccgcgcggagactgacgggacgagctggtgttgtaggaggagcagcaggagatcgcgcggtccaaaggctcctccggcgagcccctgacgtgggcggacaccaggcggatgcgcacgacgagccgtgtgatccaggagacgatgcgggtggcgtccatcctgtccttcaccttccgggaggccgtggaggacgtggagtaccaagggtacctgatccccaagggctggaaggtgatgcccctgttccggaacatccaccacagccccgaccacttcccctgcccggagaagttcgacccctcccgattcgaggtcagactctctctgcctctgcttctgcttcgccatttgtgccatgtcagacaggtcctcatggctggcctctgtttcaggttgctcccaagcccaacacgttcctgccgttcggcaacgggacccactcgtgcccgggcaacgagctcgccaagctggagatgctcgtgctcttccaccacctcgccaccaagtacaggtggtccacctccaagtccgagagcggcgtgcagttcggccccttcgcgctgccgctcaacggcctgcccatgaccttcgtccgcaaggactga

SEQ ID NO: 82 - protein, 528 aa, Zea mays
MGAFLLFVCLLAPVVVLACAVRGRKRRASSAAAGGKALPLPPGSMGWPYVGETFQLYSSKNPNVFFARKQNRYGPIFKTHILGCPCVMVSSPEAARFVLVTQAHLFKPTFPASKERMLGPQAIFFQQGDYHAHLRRLVSRAFSPEAIRGSVPAIEAIALRSLESWDGRLVNTFQEMKLYALNVALLSIFGEEEMRYIEELKQCYLTLEKGYNSMPVNLPGTLFHKAMKARKRLGAVVAHIIEARREGERQRGSDLLASFLDDREALTDAQIADNVIGVIFAARDTTASVLTWMVKFLGDHPAVLKAVIVSSSLPAGSCLAPDRLRAPAARRLTGRAGVVGGAAGDRAVQRLLRRAPDVGGHQADAHDEPCDPGDDAGGVHPVLHLPGGRGGRGVPRVPDPQGLEGDAPVPEHPPQPRPLPLPGEVRPLPIRGQTLSASASASPFVPCQTGPHGWPLFQVAPKPNTFLPFGNGTHSCPGNELAKLEMLVLFHHLATKYRWSTSKSESGVQFGPFALPLNGLPMTFVRKD

SEQ ID NO: 83 - DNA, 1437 bp, Sorghum bicolor
atgggcgccttgttgctcttcgtctgcctcctggcgccgatcgtgctcttgtgcgccgccgtccgcggcagccggaagcggcggtccgcctcgccggcgtcgtcgtgcggcaaggcgctgcctctgccgccggggtcgatgggttggccgtacgtgggcgagacgttccagctctactcgtccaagaaccccaacgtgttcttcgcccggaagcagaaccggtacgggcccatcttcaagacccacatcctgggttgcccctgcgtgatggtgtccagccctgaggcggcgcgcttcgtgctcgtcacgcaggcgcacctcttcaagcccacgttcccggcgagcaaggagcgcatgctggggccacaggccatcttcttccagcagggcgactaccacacccacctccgccgcctcgtctcccgggctttctcccccgaggccatccgcggctccgtgccggccatcgaggccgtcgcgctgcgctcgctcgactcctgggacggacaactcgtcaacaccttccaagagatgaagctgtacgcgctgaatgtggcattgctgtccatcttcggcgaggaggagatgcggtacatcgaggagctgaagcagtgctacctgacgctggagaaagggtacaactcgatgccggtgaacctgccgggcaccctgttccacaaggccatgaaggcccggaagcggctgggcgccatcgtggcccacatcatcgaggcccggcgcgggcggcagcagcagcagcagcagcagcagcgcgggagggacctcctggcgtcgttcctggacgaccgcgaggcgctgacggacgcccagatcgcggacaacgtgatcggggtcatcttcgcggcccgcgacaccaccgccagcgtgctcacctggatggtcaagttcctcggcgacaaccccgcggtgctcaaggcggtcatcgaggagcagcaggagatcgcgcggtccaaggggtcctcctccgacgagcccctgacgtgggcggacacgaggcggatgcgcatgacgagccgtgtgatccaggagaccatgcgggtggcgtccatcctgtccttcaccttccgggaggccgtggaggacgtggagtaccaagggtacctgatccccaagggctggaaggtgatgcccctgttccggaacatccaccacagccccgaccacttcccatgcccggagaagttcgacccctcccgattcgaggttgctcccaagcccaacacgttcatgccgttcggcaacgggacccactcgtgcccgggcaacgagctcgccaagctggagatgctggtgctcttccaccacctcgccaccaagtacaggtggtccacctccaagtccgagagcggcgtgcagttcggccccttcgcgctgccgctcaacggcctgcccatgaccttccttcgcaaggactga

SEQ ID NO: 84 - protein, 478 aa, Sorghum bicolor
MGALLLFVCLLAPIVLLCAAVRGSRKRRSASPASSCGKALPLPPGSMGWPYVGETFQLYSSKNPNVFFARKQNRYGPIFKTHILGCPCVMVSSPEAARFVLVTQAHLFKPTFPASKERMLGPQAIFFQQGDYHTHLRRLVSRAFSPEAIRGSVPAIEAVALRSLDSWDGQLVNTFQEMKLYALNVALLSIFGEEEMRYIEELKQCYLTLEKGYNSMPVNLPGTLFHKAMKARKRLGAIVAHIIEARRGRQQQQQQQQRGRDLLASFLDDREALTDAQIADNVIGVIFAARDTTASVLTWMVKFLGDNPAVLKAVIEEQQEIARSKGSSSDEPLTWADTRRMRMTSRVIQETMRVASILSFTFREAVEDVEYQGYLIPKGWKVMPLFRNIHHSPDHFPCPEKFDPSRFEVAPKPNTFMPFGNGTHSCPGNELAKLEMLVLFHHLATKYRWSTSKSESGVQFGPFALPLNGLPMTFLRKD

SEQ ID NO: 85 - DNA, 1449 bp, Arabidopsis thaliana
atgcaaatctcatcttcatcgtcttcaaatttcttctcttctctttatgctgatgaaccggcactaatcacattaacaattgttgtagtagtagtagtgttactatttaaatggtggttgcactggaaagagcaaagactacggctacctcctggctccatggggttgccttacatcggagagacactccgcctctacacagaaaatcccaattccttcttcgccactcgccaaaacaagtacggggatatattcaagacgcacatattaggatgtccatgtgtgatgataagtagtccagaggcggctcgaatggtgttagtgagcaaagctcacttgttcaagccaacttatcctccaagcaaagagcgtatgattggaccagaggctcttttcttccaccaaggtccataccattctacccttaagcggctggtccagtcttctttcatgccttctgctctcagaccaaccgtctctcacatcgagctccttgtcctccaaaccctttcctcttggacgtcccaaaagtccatcaacaccctcgaatacatgaaacgatatgcattcgatgtggcgatcatgtcagcgttcggggacaaagaggagcccactacgattgatgttattaagcttctctatcaacgtctcgaaaggggttacaactccatgcctctcgacctaccgggcacactttttcataagtccatgaaggcaagaatagaattaagcgaggaactaaggaaagtaatagagaagagaagagagaatgggagagaagaaggaggactattgggagtacttctgggagcaaaggatcaaaaacgcaacggcttaagtgattcacagattgctgacaacatcatcggtgttatattcgccgccaccgacaccaccgcttctgtcttaacttggcttctcaagtacttacacgaccaccccaatctcctccaagaagtctccagggagcaattcagcattcgacagaaaataaaaaaagaaaaccgaagaatctcatgggaagatacaagaaaaatgccactgaccactagggtgatacaagagacactaagagcagcaagtgtactgtcctttacatttagagaagcagtacaagacgtcgaatatgatggctacttgatcccaaagggttggaaggttcttcctcttttccggcgaatccatcactcctccgaattcttccccgatcctgaaaaattcgatccttctagattcgaggtggcaccaaaaccttacacgtacatgccattcggaaatggagtgcactcatgtccaggaagtgagctggctaaacttgagatgcttatcctccttcaccacctcactacttccttcagatgggaagtgattggagatgaagaaggtatacagtatggtcctttccctgtacccaagaagggtttaccaataagagtaaccccgatttaa

SEQ ID NO: 86 - protein, 482 aa, Arabidopsis thaliana
MQISSSSSSNFFSSLYADEPALITLTIVVVVVVLLFKWWLHWKEQRLRLPPGSMGLPYIGETLRLYTENPNSFFATRQNKYGDIFKTHILGCPCVMISSPEAARMVLVSKAHLFKPTYPPSKERMIGPEALFFHQGPYHSTLKRLVQSSFMPSALRPTVSHIELLVLQTLSSWTSQKSINTLEYMKRYAFDVAIMSAFGDKEEPTTIDVIKLLYQRLERGYNSMPLDLPGTLFHKSMKARIELSEELRKVIEKRRENGREEGGLLGVLLGAKDQKRNGLSDSQIADNIIGVIFAATDTTASVLTWLLKYLHDHPNLLQEVSREQFSIRQKIKKENRRISWEDTRKMPLTTRVIQETLRAASVLSFTFREAVQDVEYDGYLIPKGWKVLPLFRRIHHSSEFFPDPEKFDPSRFEVAPKPYTYMPFGNGVHSCPGSELAKLEMLILLHHLTTSFRWEVIGDEEGIQYGPFPVPKKGLPIRVTPI

SEQ ID NO: 87 - DNA, 1440 bp, Glycine max
atgcaagctattattatttctttccttctcatcatcacatccctgcttttcagttctttctttcttcttcttttccctttcctccactgctggcaccaccacaagcacaaaaaattgcctcctggttccatgggttggccttatctaggagagaccctcaagctctacactcaaaacccaaattccttcttctctaaccgacaaaaacggtatggagatatattcaagacaaacatattggggtgtccttgtgtgatgatatcaagccctgaggccgctagaattgtgcttgtgactcaagcacatctcttcaagccaacataccctccaagcaaagagaagttgatagggccagaggctgtgttctttcaacaaggtgcctatcactccatgctcaagaggttggttcaagcctcttttttaccctccacaattaagcactcagtctctgaggtcgagcgaattgtcatcaaaatggtgccaacttggacctacaaaactatcaacaccttgcaagagatgaaaaagtatgcatttgaagtagctgcaatctcagcttttggggaaataaaggagcttgaaatggaagaaatcagggagctctatcgttgcttggagaagggatacaactcttatccattaaatgttcctggaacttcctattggaaggcaatgaaggcaaggaggcatttgaatgagagcataaggaggataatagagagaagaaaggaaagttcaaattatggtggggggctattgggagttctattgcaagctcgaggtgagaagaacaacaagtactatcagcagctcacagattctcaagttgctgataatctcattggtgtcatctttgctgcacatgacaccacagcaagtgctctaacatgggtcctcaagtacttgcacgacaacgccaatctattggaagctgtgacgaaagaacaagaaggaataaaaaacaaactagctatggaaaatcgtggactttcgtgggatgataccaggcagatgccgttcactagccgggtgatccaagaaacactgagaagtgcaagcattttgtcattcacattcagagaagcagtaacagatgttgagttggaaggttacactattccaaaaggttggaaggtccttcccctcttcagaagcattcatcattctgctgacttcttccctcagccagagaagtttgacccttcaagattcgaggtgccaccgagaccaaacacatacatgccttttggaaatggagtccactcttgtccaggcagtgagctggctaagcttgagcttcttgtcctccttcatcatcttaccctttcttacaggtggcaagttgtgggaaatgaagatggaattcaatatggtccttttccagtgcccaaacatgggttaccagtgaagataaccccgaggaacaagatatttacgtga

SEQ ID NO: 88 - protein, 479 aa, Glycine max
MQAIIISFLLIITSLLFSSFFLLLFPFLHCWHHHKHKKLPPGSMGWPYLGETLKLYTQNPNSFFSNRQKRYGDIFKTNILGCPCVMISSPEAARIVLVTQAHLFKPTYPPSKEKLIGPEAVFFQQGAYHSMLKRLVQASFLPSTIKHSVSEVERIVIKMVPTWTYKTINTLQEMKKYAFEVAAISAFGEIKELEMEEIRELYRCLEKGYNSYPLNVPGTSYWKAMKARRHLNESIRRIIERRKESSNYGGGLLGVLLQARGEKNNKYYQQLTDSQVADNLIGVIFAAHDTTASALTWVLKYLHDNANLLEAVTKEQEGIKNKLAMENRGLSWDDTRQMPFTSRVIQETLRSASILSFTFREAVTDVELEGYTIPKGWKVLPLFRSIHHSADFFPQPEKFDPSRFEVPPRPNTYMPFGNGVHSCPGSELAKLELLVLLHHLTLSYRWQVVGNEDGIQYGPFPVPKHGLPVKITPRNKIFT

SEQ ID NO: 89 - DNA, 969 bp, Oryza sativa
atgagcggcggcggcgaaggggcggcggcggcggagaggcaggagctgcagctgccgccggggttcaggttccacccgacggacgaggagctggtgatgcactacctctgccggcggtgcgccggcctccccatcgccgtccccatcatcgccgaggtcgacctctacaagttcgatccatggcatctcccaagaatggcgctgtacggcgagaaggagtggtacttcttctcccctcgggaccgcaagtacccgaacgggtcgcggccgaaccgcgccgccgggtccgggtactggaaggccaccggcgccgacaagccggtgggcacgccgaggccggtggccatcaagaaggcgctcgtcttctacgccggcaaggcgcccaagggcgacaagaccaactggatcatgcacgagtaccgcctcgccgacgtcgaccgctccgcccgcaagaagaacaccctccggctagatgattgggtcctgtgccgaatctacaacaagaaaggcggcgtggagaagccgagcggcggcggcggcggcgaacgttcgaatatgatgagccacggggagaccgcgtcggcgggctcgccgccggagcagaagccggccgtgctgccgccgccgccaccgccgtacgcggcggcggcgccgttctcggagctggcggcgttctacgacgtgcggccgtcggactcggtgccgcgggcgcacggcgcggactcgagctgctcggagcacgtgctgacgacgtcggcgtcgtccggcggcgtcgtcgagcggccggaggtgcagagccagcccaagatcgccgagtgggagcgcacgttcgccggcgccgccgccccggctggcgccgtcagcacggccggaccgattctgggccagctcgaccccgccgccgccgtcgccggcggcggcgacccgctcctccaggacatcctcatgtactggggcaagccgttctga

SEQ ID NO: 90 - protein, 322 aa, Oryza sativa
MSGGGEGAAAAERQELQLPPGFRFHPTDEELVMHYLCRRCAGLPIAVPIIAEVDLYKFDPWHLPRMALYGEKEWYFFSPRDRKYPNGSRPNRAAGSGYWKATGADKPVGTPRPVAIKKALVFYAGKAPKGDKTNWIMHEYRLADVDRSARKKNTLRLDDWVLCRIYNKKGGVEKPSGGGGGERSNMMSHGETASAGSPPEQKPAVLPPPPPPYAAAAPFSELAAFYDVRPSDSVPRAHGADSSCSEHVLTTSASSGGVVERPEVQSQPKIAEWERTFAGAAAPAGAVSTAGPILGQLDPAAAVAGGGDPLLQDILMYWGKPF

SEQ ID NO: 91 - DNA, 888 bp, Zea mays
atgagcggcgccggtccggatctgcagctgccaccggggttccggttccacccgacggacgaggagctggtgatgcactacctctgccgccgctgcgccggcctgcccatcgccgtccccatcatcgccgagatcgacctctacaagttcgacccatggcagctcccaaggatggcgctgtacggcgagaaggagtggtacttcttctccccgcgggaccgcaagtacccgaacgggtccaggcccaaccgcgccgccggggctgggtactggaaggccaccggcgctgacaagcccgtgggcacgcccaagccgctggccatcaagaaggcgctcgtcttctacgccggcaaggcgcccaagggcgagaagaccaactggatcatgcacgagtaccgcctcgccgacgtcgaccgctcggcgcgcaagaagaacagcctcaggttggatgactgggtcctgtgccgcatctacaacaagaagggcggcgggctggagaaggcggcggcgccggcggccggcggcgaccacaagcctgtgttcgccacggcggcggtgagctccccgccggagcagaagccgttcgtggcggcggcgggcgggctgcccccggcgttcccggagctggcggcgtactacgaccggccgtcggactcgatgccgcggctgcacgcggactactccagctgctcggagcaggtgctgtccccggagcagctggcgtgcgaccgggaggtgcagagccagcccaagatcagcgagtgggagcggaccttcgcctccgaccccgtgaaccccgcgggctccatgctcgaccccgtcgtcggccacgccggcggcgacccgctgctgcaggacatcctcatgtactggggcaagccgttctag

SEQ ID NO: 92 - protein, 295 aa, Zea mays
MSGAGPDLQLPPGFRFHPTDEELVMHYLCRRCAGLPIAVPIIAEIDLYKFDPWQLPRMALYGEKEWYFFSPRDRKYPNGSRPNRAAGAGYWKATGADKPVGTPKPLAIKKALVFYAGKAPKGEKTNWIMHEYRLADVDRSARKKNSLRLDDWVLCRIYNKKGGGLEKAAAPAAGGDHKPVFATAAVSSPPEQKPFVAAAGGLPPAFPELAAYYDRPSDSMPRLHADYSSCSEQVLSPEQLACDREVQSQPKISEWERTFASDPVNPAGSMLDPVVGHAGGDPLLQDILMYWGKPF

SEQ ID NO: 93 - DNA, 876 bp, Sorghum bicolor
atgagcggcggcggtcaggatctgcagctgccgccggggttccggttccacccgacggacgaggagctggtgatgcactacctctgccgccgctgcgccggcctgcccatcgccgtccccatcatcgccgagatcgacctctacaagttcgacccatggcagctccccaggatggcgctgtacggcgagaaggagtggtacttcttctccccgcgggaccgcaagtacccgaacgggtcgaggccgaaccgcgccgccgggtccggctactggaaggccaccggcgccgacaagcccgtgggcacgcccaagccgctcgccatcaagaaggcgctcgtcttctacgccggcaaggcgcccaagggcgagaagaccaactggatcatgcacgagtaccgcctcgccgacgtcgaccgctccgcccgcaagaagaacagcctcaggttggatgactgggtcctgtgccgcatctacaacaagaagggcgggctggagaagccgtcggcggtcgccggcggcgaccacaagccgatgttcgcagcggccgcggtgagctccccaccggagcagaagccgttcgtggcggcgccgggcgggcttcccccattcccggacctggcggcgtactacgaccggccgtcggactcgatgccgcggctgcacgcggactccagctgctcggagcaggtgctgtcgccggagcagctggcgtgcgaccgggaggtgcagagccagcccaagatcagcgagtgggagcgcaccttcgcctccgaccccgtcaaccccgccggctccatgctcgaccccgtcgtcggtggccacgccggcgacccgctgctgcaggacatcctcatgtactggggcaagccgttctag

SEQ ID NO: 94 - protein, 291 aa, Sorghum bicolor
MSGGGQDLQLPPGFRFHPTDEELVMHYLCRRCAGLPIAVPIIAEIDLYKFDPWQLPRMALYGEKEWYFFSPRDRKYPNGSRPNRAAGSGYWKATGADKPVGTPKPLAIKKALVFYAGKAPKGEKTNWIMHEYRLADVDRSARKKNSLRLDDWVLCRIYNKKGGLEKPSAVAGGDHKPMFAAAAVSSPPEQKPFVAAPGGLPPFPDLAAYYDRPSDSMPRLHADSSCSEQVLSPEQLACDREVQSQPKISEWERTFASDPVNPAGSMLDPVVGGHAGDPLLQDILMYWGKPF

SEQ ID NO: 95 - DNA, 762 bp, Arabidopsis thaliana
atgatgaaatctggggctgatttgcaatttccaccaggatttagatttcatcctacggatgaggagctagtcctcatgtatctctgtcgtaaatgcgcgtcgcagccgatccctgctccgattatcaccgaactcgatttgtaccgatatgatccttgggaccttcccgacatggctttgtacggtgaaaaggagtggtattttttctcaccaagagatcgaaagtatccaaacggttcaagacccaaccgtgcagctggtactggatattggaaagctaccggagctgataaaccaataggtcgtcctaaaccggttggtattaagaaggctctagtgttttactcgggaaaacctccaaatggagagaaaaccaattggattatgcacgaataccggctcgctgacgttgaccggtcggttcgtaagaaaaacagtctaagattggacgattgggtattgtgtcgtatatataacaagaaaggtgtcatcgagaagcgacgaagcgatatcgaggacgggttaaagcctgtgactgacacgtgtccaccggaatctgtggcgagattgatctccggctcggagcaagcggtgtcaccggaattcacgtgtagcaacggtcggttgagtaatgcccttgattttccgtttaattacgtagatgccatcgccgataacgagattgtgtcacggctattgggcgggaatcagatgtggtcgacgacgcttgatccacttgtggttaggcagggaactttctga

SEQ ID NO: 96 - protein, 253 aa, Arabidopsis thaliana
MMKSGADLQFPPGFRFHPTDEELVLMYLCRKCASQPIPAPIITELDLYRYDPWDLPDMALYGEKEWYFFSPRDRKYPNGSRPNRAAGTGYWKATGADKPIGRPKPVGIKKALVFYSGKPPNGEKTNWIMHEYRLADVDRSVRKKNSLRLDDWVLCRIYNKKGVIEKRRSDIEDGLKPVTDTCPPESVARLISGSEQAVSPEFTCSNGRLSNALDFPFNYVDAIADNEIVSRLLGGNQMWSTTLDPLVVRQGTF

SEQ ID NO: 97 - DNA, 903 bp, Glycine max
atggcatcggagcttcaattgcccccaggcttcagattccatccaacggaccaggagctggtgttgcactatctctgccgtaaatgcgcatcgcagcctatcgccgttcccatcatcgccgaaatcgacctctacaaatacgacccctgggacctacccggattggcttcctacggagagaaagagtggtacttcttttcaccacgggaccggaaatacccgaacggttccaggccgaaccgggcggcgggaaccggttactggaaggcaaccggggcggataagcccattggccaccccaaaccggttgggataaaaaaagctttggtgttttacgcagggaaagctccgaaaggggacaagagcaattggatcatgcacgagtatcgtctcgccgatgtagatcgctccgttcgcaaaaagaacagcctaaggttagatgattgggtgctttgccgtatttacaacaagaagggcacgatcgagaagttccaaccaagcagcgatgttgttgttagccgaaaaatggaatcatcggagatcgaagacaggaagccggagattctgaaaagcggaggaggttgtcttctgccgccggttccgccgccgcaagcgaaggcggcggtgaagaaggattacatgtacttcgacccgtcggattcaatcccgaagctgcacacggactcgagctgttcggagcacgtggtatcgccggaattcgcgagcgaggtgcagagcgagccaaagtggaaggagtgggagaaaagcctcgagtttccgtttaattacgtggatgccactctgaacaacagcaacagcttcacgacgcaattccagggcaataatcagatgatgtcgccgctgcaggacatgttcatgtactggcccaacaagcccttctga

SEQ ID NO: 98 - protein, 300 aa, Glycine max
MASELQLPPGFRFHPTDQELVLHYLCRKCASQPIAVPIIAEIDLYKYDPWDLPGLASYGEKEWYFFSPRDRKYPNGSRPNRAAGTGYWKATGADKPIGHPKPVGIKKALVFYAGKAPKGDKSNWIMHEYRLADVDRSVRKKNSLRLDDWVLCRIYNKKGTIEKFQPSSDVVVSRKMESSEIEDRKPEILKSGGGCLLPPVPPPQAKAAVKKDYMYFDPSDSIPKLHTDSSCSEHVVSPEFASEVQSEPKWKEWEKSLEFPFNYVDATLNNSNSFTTQFQGNNQMMSPLQDMFMYWPNKPF

SEQ ID NO: 99 - DNA, 903 bp, Oryza sativa
atgagggagctgtcgtgcttcggcgacagctcggtcggcatcgccgccgccgcggccggtgactccggcggcggcggcggcggcgcgctggatcgctcgctgcaggcggcgaccacgacggtgtacggtgcgtcgctgcactccgggaaggagctcctcatccgggtcacgtggacgcggagcgccgccggagccaccggcctcgccgtcgccttcgacgacgcgctctcgccgtcgtcgaggtgcgcccaccacgtgctgcacaagaagcgcgggagccggtccctcgccaccgccgccggcacggccgtgggcgtccactgggacaccgccgaggccacgtacgcgtcgggttcgtcccccgagcccaccggcgactactacctcgccgtcgtcgccgacgccgagctcgcgctgctcctcggcgagggcggcgcggcgcgggacctctcccgccggttcggcgacgacggcggtggcgccgtcgtcctcagccggcgagagcagctgcgcggcgcggcgacggcgcacaccacgcggtgcaggttccgggagggcggggcggagcacgaggtggcggtgcacgcgacccgcggcggcggcggcggcggtgagggggaggtgcgggtcagcatcgacgggaagagggtggctgaggtgaggagggtggggtgggggttccgcggcaaccgcgccgccgtgctcgccgacggcgaggtggtggacgtgatgtgggacgtgcacgactggtggttcggccgtggcggcggcggcggcggagctggagctggcgcgcagttcatggtgagggcgagggcggagaaggaggggaggctgtggatggccgaccagccgccggcgaggggtggcttcttcctgcacgtgcaatgctaccgccggtga

SEQ ID NO: 100 - protein, 300 aa, Oryza sativa
MRELSCFGDSSVGIAAAAAGDSGGGGGGALDRSLQAATTTVYGASLHSGKELLIRVTWTRSAAGATGLAVAFDDALSPSSRCAHHVLHKKRGSRSLATAAGTAVGVHWDTAEATYASGSSPEPTGDYYLAVVADAELALLLGEGGAARDLSRRFGDDGGGAVVLSRREQLRGAATAHTTRCRFREGGAEHEVAVHATRGGGGGGEGEVRVSIDGKRVAEVRRVGWGFRGNRAAVLADGEVVDVMWDVHDWWFGRGGGGGGAGAGAQFMVRARAEKEGRLWMADQPPARGGFFLHVQCYRR

SEQ ID NO: 101 - DNA, 1011 bp, Zea mays
atgaggtgtgaggaaaaggagggaaagcattggcgggcgggcgggcgggcgcctgtctcttgcggctgtggtgccgcccgagagcgcgacccattcgttcgcgagcctcacttccagttccagcacgaatcctcgtccaatccacgcctgctgctcgcgagccatcgccacagtgctggcctggccatgcctggagccgttgcgcttgcgcccacgttcgcggcagcgagcagctccgccgccgcggcctccacgctggctccgaacccgacaagccgaggcgacccgctaattattaggttgtgccgcaatgctcctgctagcgcaactccacttgttccacttgtagccacccgtcgctacgtgcgtggctgccgcggcgcggctctagtggccagtccccaccacgctccaaaccctcgcctccgattcgcgtctgcggcagaggggatggctgcggaagcgagcacggcgggtgcggcgtcggcagccgaggcgaagcccttcgccgtcctcttcgtgtgcctcgggaatatttgccggagtcctgcggctgaagctgtgtttcggaccctcgtaagcaagcgtgggcttgactccaagtttctcatagactctgctggtaccatcgggtatcatgagggtaataaggcagactcaaggatgagagcagcttcaaaaaagcgggggattgaggtcacatcaatatccaggcctatcaaaccctcggattttcgtgattttgatcttatccttgcaatggacaggcagaactatgaagatatattgaactcgtttgagagatggagacgcaaagagcccctccctgatagtgcacccaataaggttaagctgatgtgctcctactgcaaacaacatactgagtctgaagttccagatccttattatggaggtcctcagggatttgaaaaggtgttggacttattggaagatgcttgcgagtcgctgcttgatagtatcgtcgcaaacaatgcaagcatttctgggtga

SEQ ID NO: 102 - protein, 336 aa, Zea mays
MRCEEKEGKHWRAGGRAPVSCGCGAARERDPFVREPHFQFQHESSSNPRLLLASHRHSAGLAMPGAVALAPTFAAASSSAAAASTLAPNPTSRGDPLIIRLCRNAPASATPLVPLVATRRYVRGCRGAALVASPHHAPNPRLRFASAAEGMAAEASTAGAASAAEAKPFAVLFVCLGNICRSPAAEAVFRTLVSKRGLDSKFLIDSAGTIGYHEGNKADSRMRAASKKRGIEVTSISRPIKPSDFRDFDLILAMDRQNYEDILNSFERWRRKEPLPDSAPNKVKLMCSYCKQHTESEVPDPYYGGPQGFEKVLDLLEDACESLLDSIVANNASISG

SEQ ID NO: 103 - DNA, 1005 bp, Sorghum bicolor
tccccttgcctgccccgagcgaggaagggcacacgcaatgcgagatttctcctgtttcggcgacgccgccgtcacgctggccgccggggcagctggcggcggcgggggaggaggaggcgccgccgccgcgctcgaccgctcgctccaggcggccacggccagcgactacagggtcgcgctgtcgtcgcgcaaggagctccggatcaaggtcacctggacgcggggagtcgtcgcgggggccagcggcgcggtggctggcgcggcgggggggccgaccgggatcgcgctggccatcgacgacgggtcgtccctggcggcgcctcctccgctggcggctgtggcgctgatcggcacgcagcgccggacggccccggcgcccgcgcccgcgcagcacttcctgcagaagaagcgcgggacccggtccttcgtcaccgacgccggcacggcggtgtccatctactgggacacggcggaggccaagtactgccccccgggcgcggcggagccctcccgcgactaccacctcgccgtggtcgcggacggcgagctcgcgctgctgctcggcggcggcgtcggaggcgaggcggcgcgcgacgtccggcgccgctacgcgcccgcgccgcgccgcgcgctgctcagccgccgcgagcaggtccgcgggcccttctcctcctcctcctcctctgctcccccggcgcatcagctggtccacacgacgcgctgcaggttccgcgacgacggcgccgagcacgacgtcacggtcgcgtgccgcggggacgagtgggggtccagggacggcgaggtgtccgtcagcgtcgacggcaagaaggtggtggaggcgcgccgggtcaagtggaacttccgcggcaaccggaccgccgtgttgggcgacggcgccgttgtcgaggtcatgtgggacgtgcacgactggtggttcgccggcgtgcacggcggcggcggcggcggcggcgcgcagttcatggtcaaggcgcgcggggctgctga

SEQ ID NO: 104 - protein, 334 aa, Sorghum bicolor
SPCLPRARKGTRNARFLLFRRRRRHAGRRGSWRRRGRRRRRRRARPLAPGGHGQRLQGRAVVAQGAPDQGHLDAGSRRGGQRRGGWRGGGADRDRAGHRRRVVPGGASSAGGCGADRHAAPDGPGARARAALPAEEARDPVLRHRRRHGGVHLLGHGGGQVLPPGRGGALPRLPPRRGRGRRARAAARRRRRRRGGARRPAPLRARAAPRAAQPPRAGPRALLLLLLLCSPGASAGPHDALQVPRRRRRARRHGRVPRGRVGVQGRRGVRQRRRQEGGGGAPGQVELPRQPDRRVGRRRRCRGHVGRARLVVRRRARRRRRRRRAVHGQGARGC

SEQ ID NO: 105 - DNA, 870 bp, Arabidopsis thaliana
atgtttaatcaatccagctctgtctcgttaatctacgtagttgaaatcgccaaaacaccacaaaacgtagacgtcacttggtctaaaaccacctcctcacattctttaaccatcaaaatcgagaacgtcaaagacgagcaacagaatcatcatcaaccggtgaagatagatctttcaggttcttcgttttgggccaaaaagggtctcaagagcttagaagctaacggaactagagtcgacgtatactgggattttcgtcaagccaaattctcgaacttccctgaaccttcctctggcttctacgtctctctcgtatcccaaaacgcaaccgttttaacgatcggggatttaaggaacgaagctttaaagaggacgaagaagaacccttcagctacagaagctgccttggtctccaagcaagaacacgtccacgggaaacgcgttttctacacgcggacggcgtttggcggtggggagtcgaggcgggagaatgaggtggtgatcgaaacatctctgtcgggtcctagcgatccagagatgtggatcacggtggacggtgtgccggcgattaggatcatgaatttgaattggagatttagagggaatgaggttgtgactgtgagtgatggtgtttctttggagatcttttgggacgttcatgattggctgtttgaaccctctggttcgtctagtgggttgtttgttttcaagcctaaagctggatttgaatctaaatgtcttagttttaatggtggctatggtgatggtgaaggtgaggatcatgatgtggaagatgacgattcgtcgcccaagtattgtcatgtcctatatgccgtcaaagaactagaatttccatgtcaaaaaaattag

SEQ ID NO: 106 - protein, 289 aa, Arabidopsis thaliana
MFNQSSSVSLIYVVEIAKTPQNVDVTWSKTTSSHSLTIKIENVKDEQQNHHQPVKIDLSGSSFWAKKGLKSLEANGTRVDVYWDFRQAKFSNFPEPSSGFYVSLVSQNATVLTIGDLRNEALKRTKKNPSATEAALVSKQEHVHGKRVFYTRTAFGGGESRRENEVVIETSLSGPSDPEMWITVDGVPAIRIMNLNWRFRGNEVVTVSDGVSLEIFWDVHDWLFEPSGSSSGLFVFKPKAGFESKCLSFNGGYGDGEGEDHDVEDDDSSPKYCHVLYAVKELEFPCQKN

SEQ ID NO: 107 - DNA, 906 bp, Glycine max
atgaacatgtcagatatgatatcttgtttcaacgagaacgcagtgaatgtgtcacactcctcatgttctagctactcaaacaacgcttgcatatctccaagtgttacaccttcaactcaaaattcagtgtcttctgtctacaaaaccaccctctcaaaccaaaagcagcttctgatcacagtcacgtggtgcaagagccactccaaccaaggactcaacgtaaccttcggcgaagagaacaacaaccctttggcaccatctttcagactcaacaccaattcacgctttttcaggaaaaagaaaggaagcaaaatgttggaatccgaagactcaaaagttgaagtcttctgggacctctcgaaggccaagtatgacactggccctgaacctgttgaagggttttacgtggcgattctcgttgatgcagaaataggcctcattctcggtgaagatgtggccaagaagttcaaaacaagaacccttttgggcaatgtttcgctgttatcacggcgtgagcattgctcgggtaacgccgtttacgcaaccaaggctcagttttgtgacactggaacttggcatgacattttgatcagatgcagtggcgagaatgaaggactcaaagctcctgttttgtctgtttgcattgacaagaagacggtgattcgtgtgaagaggctgcagtggaatttcaggggcaaccaaacgattttcgtcgatgggttgcttgtggatttgctttgggatgttcataactggtttttcaaccctgcttctgggaatgctgtgttcatgttcaggaccaggagtggcttggatagcagattgtggttagaggagaagattgcacagaaagataaagatagagttgaattctccttgttgatctatgcctataagaacacatga

SEQ ID NO: 108 - protein, 301 aa, Glycine max
MNMSDMISCFNENAVNVSHSSCSSYSNNACISPSVTPSTQNSVSSVYKTTLSNQKQLLITVTWCKSHSNQGLNVTFGEENNNPLAPSFRLNTNSRFFRKKKGSKMLESEDSKVEVFWDLSKAKYDTGPEPVEGFYVAILVDAEIGLILGEDVAKKFKTRTLLGNVSLLSRREHCSGNAVYATKAQFCDTGTWHDILIRCSGENEGLKAPVLSVCIDKKTVIRVKRLQWNFRGNQTIFVDGLLVDLLWDVHNWFFNPASGNAVFMFRTRSGLDSRLWLEEKIAQKDKDRVEFSLLIYAYKNT

SEQ ID NO: 109 - DNA, 1335 bp, Oryza sativa
atggacccgtgcccgttcgtgcgggtgctggttggcaacctggcgctgagaatgccggtggcgccgccggcggcgggtgcgggggcgggggtccacccgtcgaccgcgccgtgctactgcaagatccggctcgggaggatgccgtggcaggtcgccgcggcgccgctggtggttgccgatggtggggagcaggcgccgtcgggggcgctggccgccgcgttccatctgtccaaggcggatttggagtggttcgcgcggaagccgtcgctgctgttctcgtcgtcgtcgtcgtctcgcgggccggcgacgctgaaggtggcggtgtacgccgggaggaaggggacgacgtgcggtgttagctctgggcggttgattgggaaggctaccattccggtggatctcaagggcgccgaggcgaaggccgcggtggtgcatagcggctggatctgtgttgggaagaagagcggcggcaagggcggctccgcggcggcggagctcagcctcaccgtgcgcgcggagcccgatccgaggttcgtgttcgagttcgacggcgagccggagtgcagcccgcaggtgctgcaggtgaggggaagcatgaagcagccgatgttcacctgcaagttcgggtgccgcagcaacagcgacctgcggagatcggtggttcagacggagcgggacgccgccgccgccgccgggaaggagcggaaggggtggtcggtgacggtgcacgacctgtcggggtcccccgtggcgctggcgtcgatggtgacgccgttcgtggcgtcgccggggacggaccgggtgagccgctccaacccgggcgcgtggctcatcctccgccccgccggcgacgggtcgtgggagccatggggccgcctcgagtgctggcgagagcgcggcggcgcgggcgcctccaacagcctcggctaccgcttcgacctcctcctcccgggcgtcgaccacgccgtccccttggcggagtcctccatcgccgcttccaagggcggcaagttcgccatcgacctcacctcgatgcagccccagagccggggcggcacgccggggtgcagcccgcggggcagcggcgacttcagccagtggccgctcgccagctacagctaccgcggcttcgtgatgtcctcctccgtccagggcgaggggcggtgcagcaagcccacggtggaggtcggcgtcccgcacgtcggctgcgccgaggacgcggccgcgttcgtggcgctcgccgccgcggtcgacctcagcatggacgcgtgcaggctgttctcccacaagctcaggaaggagctctcccacctgcgctccgacgtgctcaggtga

SEQ ID NO: 110 - protein, 444 aa, Oryza sativa
MDPCPFVRVLVGNLALRMPVAPPAAGAGAGVHPSTAPCYCKIRLGRMPWQVAAAPLVVADGGEQAPSGALAAAFHLSKADLEWFARKPSLLFSSSSSSRGPATLKVAVYAGRKGTTCGVSSGRLIGKATIPVDLKGAEAKAAVVHSGWICVGKKSGGKGGSAAAELSLTVRAEPDPRFVFEFDGEPECSPQVLQVRGSMKQPMFTCKFGCRSNSDLRRSVVQTERDAAAAAGKERKGWSVTVHDLSGSPVALASMVTPFVASPGTDRVSRSNPGAWLILRPAGDGSWEPWGRLECWRERGGAGASNSLGYRFDLLLPGVDHAVPLAESSIAASKGGKFAIDLTSMQPQSRGGTPGCSPRGSGDFSQWPLASYSYRGFVMSSSVQGEGRCSKPTVEVGVPHVGCAEDAAAFVALAAAVDLSMDACRLFSHKLRKELSHLRSDVLR

SEQ ID NO: 111 - DNA, 1314 bp, Zea mays
atggacccgtgcccgttcgtgcgggtgctggtgggcaacctcgcgctcagaatgccggtggcgccgcccgcctccggggccggcgcgggcgtccacccgtccacgtcggcgtgctactgcaagatccggctcgggaagatgccggtccagagcgtcccggcgccgctcgtggtcaccgacggcggcgagcagacgccggcgtccggggcactcgccgccgcgttccacctgtcgaaggctgacctggagtggttcgacgggaagccctcgctcttctcgtcgcggcgcggggcaggggacgctagcctgaaggtgtcggtctacgccggccggaaggggagtgcctgcggcgtcagctccgggcggctgctcgggaaggctacggtcccgctcgacctcaagggcgccgaggccaagcccgccgtgctgcacagcggctggatctccatcgggaagcgggccgggaagggcagcccggcggcggcggcggagctctgcctcaccgtgcgcgcggagccggacccgcggttcgtcttcgagttcgacggcgagccggagtgcagcccgcaggtgctgcaggtgcgcggcagcatgaggcagcccatgttcacatgcaagttcgggtgccgcagcaacagcgacctgcgcaggccggggatgcggcgtgagcgcgacgccaaggagcgcaaggggtggtcggtgacggtgcacgacctgaaggggtcccccgtggcgatggcgtccatggtgacgccgttcgtgccgtcgccgggcacggaccgcgtgagccggtccaacccgggcgcgtggctcatcctccggcccgcggccgacggcgcctgggagccctgggcgcgcctcgagtgctggcgcgaccgccgcggcgccggcgcgtccgacagcctgggctaccgcttcgacctcctcgtccccggcgtggaccacgccgccgtcgccctcgccgactcctccatcccctcgtccaagggcggcaagttcgccatcgacctgaccgccgcgcagccgctcagccggggcggcacgccggggtgcagcccgagaggcagcggcgacctgagcaagtggcccctggggaactaccgcggcttcgtcatgtccgccgcggtccagggcgagggccggtgcagcaagccgacggtggaggtcggggtggcgcacgtcgggtgcgccgaggacgcggcggccttcgtcgccctcgccgcggccgtggacctgagcatggacgcgtgcaggctcttctcccaccggctgaggaaggagctctcgcacccgcaggccgacctactccggtga

SEQ ID NO: 112 - protein, 437 aa, Zea mays
MDPCPFVRVLVGNLALRMPVAPPASGAGAGVHPSTSACYCKIRLGKMPVQSVPAPLVVTDGGEQTPASGALAAAFHLSKADLEWFDGKPSLFSSRRGAGDASLKVSVYAGRKGSACGVSSGRLLGKATVPLDLKGAEAKPAVLHSGWISIGKRAGKGSPAAAAELCLTVRAEPDPRFVFEFDGEPECSPQVLQVRGSMRQPMFTCKFGCRSNSDLRRPGMRRERDAKERKGWSVTVHDLKGSPVAMASMVTPFVPSPGTDRVSRSNPGAWLILRPAADGAWEPWARLECWRDRRGAGASDSLGYRFDLLVPGVDHAAVALADSSIPSSKGGKFAIDLTAAQPLSRGGTPGCSPRGSGDLSKWPLGNYRGFVMSAAVQGEGRCSKPTVEVGVAHVGCAEDAAAFVALAAAVDLSMDACRLFSHRLRKELSHPQADLLR

SEQ ID NO: 113 - DNA, 1413 bp, Sorghum bicolor
atggacccgtgcccgttcgtgcgggtgctggtcggcaacctggcgctaaagatgccggcgtcaacaaccgcgccacgcagcaccgccgcgtccggctccggggtgcacccgaccacggcgccatgctactgccgtatccggctcaacaagctcccctaccagacggcctcggcgccgctgctgccacccaccgaggaaggcccggcgtcgtgcacgggcgccttcgccgccgcgttccacgtctccaaggccgacctggaccgcgccgccgccaagcccgcgctcctcctcggcgcccgcctccgccgccgcaccgcgcgcctcaaggtcgccgtctacgccggccgcggcggcggcgcgtcctgcggcggaggcggaggcggggtcaactccggcaggctgatcgggaagctcgtcgtcccgcttgacctcggtgctgctatggcgaagcccgtcgtcttccacagcggatgggtcgccatcggcaagcgccgctccggcgggcgtggcaagaccgcggcgagggcgcagcttaacctcaccgtccgtgctgagccggacccgaggttcgtcttcgagttcgacggcgagcctgagtgtagcccgcaggtgcttcaggtgaaggggagcatgaagcagcccatgttcacgtgcaagttctcctgccgcagcaacagcgacctccgctcgcggtccgtgcagtctgatccgggcaccgcggggccgcgcaactggctggccaagttcggttccgaccgggagcgggcggggaaggagcggaaggggtggtcagtgacggtgcacgacctctcaggctcaccggtggcactcgcatcaatggtgacgccgttcgtagcgtcccgagggacagaccgcgtgagccgctccaacccaggcgggtggctgatcctccgcccggtcgacgggacctggacaccatggggccgtctggagtgctggcgcgagcgctccggcagcggcggagggggggacaccctggggtaccgcttcgagctagtcccgggccacacgaacgcgggcgtgtgcgtggcggagtcaggcctcccggcgtcccgcggcgggcggttcgccatcgacctgacggcggcgcagccgttcgggtcgcccgggtgcagcccgcgtgggagcggcgacttgggccactaccacggcggcggggtgtggccgttcggcacgttcaggggcttcgtgatgtcggcggctgtgcagggggaaggacggtgcagcaggccgacggtggaggtcggcgtgggccacgtcgggtgcgctgaggatgccgccgcgttcgtggctctggcggccgctgttgacctcagcatggacgcgtgccggctcttctcgtgtaagcttcgccgggagctgtcggcgtctcgcgctgagctggtccggtga

SEQ ID NO: 114 - protein, 470 aa, Sorghum bicolor
MDPCPFVRVLVGNLALKMPASTTAPRSTAASGSGVHPTTAPCYCRIRLNKLPYQTASAPLLPPTEEGPASCTGAFAAAFHVSKADLDRAAAKPALLLGARLRRRTARLKVAVYAGRGGGASCGGGGGGVNSGRLIGKLVVPLDLGAAMAKPVVFHSGWVAIGKRRSGGRGKTAARAQLNLTVRAEPDPRFVFEFDGEPECSPQVLQVKGSMKQPMFTCKFSCRSNSDLRSRSVQSDPGTAGPRNWLAKFGSDRERAGKERKGWSVTVHDLSGSPVALASMVTPFVASRGTDRVSRSNPGGWLILRPVDGTWTPWGRLECWRERSGSGGGGDTLGYRFELVPGHTNAGVCVAESGLPASRGGRFAIDLTAAQPFGSPGCSPRGSGDLGHYHGGGVWPFGTFRGFVMSAAVQGEGRCSRPTVEVGVGHVGCAEDAAAFVALAAAVDLSMDACRLFSCKLRRELSASRAELVR

SEQ ID NO: 115 - DNA, 1386 bp, Arabidopsis thaliana
atggatccttgtccattcatccgtcttacaatcgggaacctagctttgaaagttccgttagcggcgaagacaacgagctccgtcgtgcatccgtcgtcttctccttgtttttgtaaaatcaaactcaaaaacttcccgccgcaaaccgccgcaatcccgtacattcctttggagacgactcagtttccggagatccaaaccctagccgccacgtttcatctcagcagctccgatattcaacgcttagcttccagatctatatttacttctaagccttgtcttaaaattttgatctacactggaagagccggcgctgcttgcggcgtacactccggtcgtcttctggcgaaagtctccgtaccgttggatctatctggtacgcaatcgaaaccgtgcgtcttccacaacggatggatatcagtcggaaaaggagctggaaaatcgtcgtcgtctgctcagtttcacctgaatgtgaaggcggagcctgatcctagattcgtttttcagtttgacggcgagcctgaatgtagtcctcaagtcgttcagattcaaggcaatatccggcaaccagttttcacatgcaaattcagttgccggcacaccggtgatcgtactcagagatcaagatcattgccgactgagacaagtgtttcacggagctggctaaactcgttcgggagtgagagagaacgtcctgggaaagagcgtaaaggatggtccataacagtccatgacttgtccggttcaccagtggccatggcgtcaatcgtcactccattcgtggcatctcctggaaccgatcgtgtgagccggtcaaaccctgggtcatggcttatactgcgtcccggagactgtacctggagaccgtggggaagacttgaagcatggcgggaacgcggtggagccactgatggtctaggttacagattcgaactcatcccagacggatcaagcggtgcaggaatcgtgcttgcggaatcaaccataagttctcacagaggtgggaaattctcaatcgagttgggatcgtcgccttcttcatcgtcgccaacaagtgtggtgaaccgatcgagaagccgtagaggtgggagtagtggaagcggtggaggagcatcgccggcgaatagtccgagaggagggagcggagattacggttacggattgtggccgtggaacgtgtacaaagggtttgtgatgtcagcaagtgtggaaggtgaagggaaatgtagtaagccttgtgtagaggtgagtgtgcagcacgttagctgtatggaagatgcggctgcttacgtggcgctttctgcagccattgatcttagtatggatgcttgcaggctgtttaatcaacggatgaggaaagagctttgccatgagtcactgagctga

SEQ ID NO: 116 - protein, 461 aa, Arabidopsis thaliana
MDPCPFIRLTIGNLALKVPLAAKTTSSVVHPSSSPCFCKIKLKNFPPQTAAIPYIPLETTQFPEIQTLAATFHLSSSDIQRLASRSIFTSKPCLKILIYTGRAGAACGVHSGRLLAKVSVPLDLSGTQSKPCVFHNGWISVGKGAGKSSSSAQFHLNVKAEPDPRFVFQFDGEPECSPQVVQIQGNIRQPVFTCKFSCRHTGDRTQRSRSLPTETSVSRSWLNSFGSERERPGKERKGWSITVHDLSGSPVAMASIVTPFVASPGTDRVSRSNPGSWLILRPGDCTWRPWGRLEAWRERGGATDGLGYRFELIPDGSSGAGIVLAESTISSHRGGKFSIELGSSPSSSSPTSVVNRSRSRRGGSSGSGGGASPANSPRGGSGDYGYGLWPWNVYKGFVMSASVEGEGKCSKPCVEVSVQHVSCMEDAAAYVALSAAIDLSMDACRLFNQRMRKELCHESLS

SEQ ID NO: 117 - DNA, 1374 bp, Glycine max
atggatccttgccctttctccagactcaccgttcgcaacctcgccctcaaaattcccgtcgcttccaaacccgcgcgctccgttgttcatccttcttcttctccctgtttctgcaaaatccagctcaagaattttcctcttcaatccgccgtcgttcccttcattcctccggattccctcttccctgactccctggtccatcctatcgctgctactttccacctcagcaagtccgatctcgacaagctcgccggcaaatccatcttctccgccaagctctgcctcaaaatctctatctacaccggccgtcgcggctccacctgcggcgtcagctccgggagactcctcggcagagtttccgttcccttggatctcaccggaacggtagccaaaaccacagtgttccacaatggatggattaggataggaaaagacgccaaaggctcttccgctcagttccatttgaatgttaaagccgaacccgatcctcgattcgtcttccagttcgacggcgaacctgaatgcagtcctcaggttttccagatccaaggcaacatttcacaacctgtcttcacctgcaagttcagtttcagaaacaacggcgaccgaaatcaccgttccaggtcgttacagtcggaaccgggaggttctagaagttggttgagttcgttcggaagcgagcgcgagcgaccggggaaggaacgcaagggatggtccataacggttcacgatctttccggttcaccggtggccgcagcttctatggtcacgcctttcgtcgcttcgcccggttcggaccgggtgagctgctccaaccctggttcgtggctaattcttcgcccgagcgacggcacgtggaagccatgggggaggctcgaggcgtggcgcgagcgcggcggctccgacggcctcggctaccgcttcgagctcataccggacaccaacggcggcatgagcgccgccggtatagtgcttgcggaatccacgctgagctccaacaaaggagggaagttcgtcatcgatttgagttgccgcaacgccgttaacggtagcggaaatggtggatctaatggccgtgcgacgccggggagcgcgacttcaccggcgtgcagcccgaggagtagtggagattatggatacggtctctggccttattgtatgtatagaggttttgtgatgtcggcgagcgtggagggtgaggggaggtgcagcaagcctactgtggaggtgagcgtgccgcacgtgaattgcacggaggatgcggcggcgtttgtggctttagcggctgccgttgatctgagcgtggatgcgtgcaggcttttctctcaacggctgaggaaggagctgtgccagcagctggatttgcttggctga

SEQ ID NO: 118 - protein, 457 aa, Glycine max
MDPCPFSRLTVRNLALKIPVASKPARSVVHPSSSPCFCKIQLKNFPLQSAVVPFIPPDSLFPDSLVHPIAATFHLSKSDLDKLAGKSIFSAKLCLKISIYTGRRGSTCGVSSGRLLGRVSVPLDLTGTVAKTTVFHNGWIRIGKDAKGSSAQFHLNVKAEPDPRFVFQFDGEPECSPQVFQIQGNISQPVFTCKFSFRNNGDRNHRSRSLQSEPGGSRSWLSSFGSERERPGKERKGWSITVHDLSGSPVAAASMVTPFVASPGSDRVSCSNPGSWLILRPSDGTWKPWGRLEAWRERGGSDGLGYRFELIPDTNGGMSAAGIVLAESTLSSNKGGKFVIDLSCRNAVNGSGNGGSNGRATPGSATSPACSPRSSGDYGYGLWPYCMYRGFVMSASVEGEGRCSKPTVEVSVPHVNCTEDAAAFVALAAAVDLSVDACRLFSQRLRKELCQQLDLLG

SEQ ID NO: 119 - DNA, 699 bp, Oryza sativa
atgtctcttgaggtcaggcaccctgcccgcccggggtgcatgctgacgcttcacggcgacgccgacgcgatggccttccagtgcaccggctgcatggaaaccggcaaaggcccaaggtacacctccggcgaccacgtcctccacacgtactgcgccctcgcgacacccacgctgcagcacccgctggtggagggtatcatggagctccggctcgtcgcccccaccggcggcgacgccgtccgctgcgacgcctgctacgacgcggtgcgagggttccactaccacagctccacgagtggcgtggacctgcacccaggttgcgccaagatgccgaggtccatcacgctgcgggggggcaccatcttcgatctccggacggaggtgtctcaccggtgcaccagctgcaaggcgatggaggggttctaccgcccatggttctaccgctccgaaaacaaccctgaccaacgcatgtacctgcacgtcaagtgcatcaaggagatccaggacgccggcgacgacgacgaggtgaggatgatggtccgcctacaagagcgtgctggccggaacgttaggctagagaggcgcgtatgcaaaacgcttgtgatcatggtgcgcatcgtcttcaggctgctcatcggggacccgacaccgatactcacagaaggagtgaacgccatcgtctccatggcgatgcagtag

SEQ ID NO: 120 - protein, 232 aa, Oryza sativa
MSLEVRHPARPGCMLTLHGDADAMAFQCTGCMETGKGPRYTSGDHVLHTYCALATPTLQHPLVEGIMELRLVAPTGGDAVRCDACYDAVRGFHYHSSTSGVDLHPGCAKMPRSITLRGGTIFDLRTEVSHRCTSCKAMEGFYRPWFYRSENNPDQRMYLHVKCIKEIQDAGDDDEVRMMVRLQERAGRNVRLERRVCKTLVIMVRIVFRLLIGDPTPILTEGVNAIVSMAMQ

SEQ ID NO: 121 - DNA, 804 bp, Sorghum bicolor
atgacgaagctgttcgaggatcccccgccggagattgcccacaccgctcacccggcgcacaagctcaagctggtcacaagcgacgacgcgcacgcggcgcccttcaagtgcgacggctgcaacgagcccggcaacgggccaaggtacacctgcgacgactgcggcagcagccacagccgaagattcgacctccacacacgctgcgcccttgcggagtcgcggaaggacaccatcgagcacccactgttccgcaaccgcgtcttcaagttccggcagcagcctccgccgcccgtcaatggaacgatctgcgacgcctgcggcgagcccgcgcacgggttcgtctaccattgctccgagaagaacaagggcggcggaggcctagacctccacccgtgctgcgcgaccctgccggagcgcatctgcaaggacggccacgccttggtgctccgcccgagcacgtcacggcggtgctgcatctgcggccaccgcgacgacggccggtactgggcgtaccgcttcgaaggcgaggatggcgtagatgacatgcacgtggcgtgcttgaagaagacggcttaccagatctgggaaacggcttacgagaaccagtaccatagcggcggcgcccagaaccttcacgtcggtctcaccgacatcgacggcctgctgcagatctgcaagaacagccagaccagcggcgggttggaccaattcatcaggatcgctggcagtgttgccagcatcatcatcgcgatcatctttgcaaatccagccgccttgatatctgcaattcctaaaaagtactaa

SEQ ID NO: 122 - protein, 267 aa, Sorghum bicolor
MTKLFEDPPPEIAHTAHPAHKLKLVTSDDAHAAPFKCDGCNEPGNGPRYTCDDCGSSHSRRFDLHTRCALAESRKDTIEHPLFRNRVFKFRQQPPPPVNGTICDACGEPAHGFVYHCSEKNKGGGGLDLHPCCATLPERICKDGHALVLRPSTSRRCCICGHRDDGRYWAYRFEGEDGVDDMHVACLKKTAYQIWETAYENQYHSGGAQNLHVGLTDIDGLLQICKNSQTSGGLDQFIRIAGSVASIIIAIIFANPAALISAIPKKY

SEQ ID NO: 123 - DNA, 1566 bp, Oryza sativa
atggcggccaccacccacgccgcctccctctccttcctcctctctcacccccaccccacctcccccaaccctaaccctaaccctaacctccccctccgccgcgccccccaccgcgtccgctgcgccaccgacgccgccgccaccaggcaccggcgcgcggccgacgagaacatccgggaggaggcggcgaggcaccgcgccccgaaccacaacttctccgcgtggtacgcgcccttcccgcccgcccccaacggcgaccccgacgagcgctactccctggacgagatcgtctaccgctccagctcgggcggcctcctcgacgtgcgccacgacatggacgcgctcgcccgcttcccgggctcctactggcgcgacctcttcgactcccgcgtcggccgcaccacctggcccttcggctccggcgtctggtccaagaaggagttcgtcctccccgagatcgaccccgaccacatcgtctccctcttcgagggcaactccaacctcttctgggcggagcgcctcggccgcgaccacctcgccgggatgaacgacctctgggtcaagcactgcggcatctcccacaccggatcgttcaaggatctcggcatgacggtgctcgtcagccaggtgaaccgcctccgccgcgcgccgctctcccgccccatcgccggagtcgggtgcgcctccacgggggacacctccgccgcgctctcggcctactgcgccgccgcggggatcccggccattgtgttcctccccgccaaccgcatctcgctcgagcagctcatccagcccattgccaatggcgccaccgtgctctcgctcgacacggacttcgacggctgcatgcggctcatcagggaggtgactgccgagctgccgatttaccttgcgaattcattgaattcccttcggctcgaggggcagaagactgctgctattgagattcttcagcagttcgattgggaggtgccggattgggtcattgttcccggaggcaatcttgggaacatatatgccttctacaagggattcgagatgtgccgcgtccttgggctcgtcgatcgtgtgccgcggcttgtctgcgcgcaggctgccaatgcgaacccgctgtaccggtactacaagtcggggtggactgagttcacgccgcaggtggctgagccgacatttgcatcggcaattcagattggtgacccggtatctgtcgatcgcgcggtggttgcgctcaaggcaactgatggcatcgtcgaggaggccacggaggaggaactcatgaacgcaatgtcgctcgctgatcgcactggcatgtttgcttgcccgcatactggggttgccctcgcagcactgttcaagctccgtgaccagcgcatcatcgggccaaatgaccgcacggtagtcgtcagcacagctcatggtctgaagttctcacagtccaagatcgactaccatgacagcaagatcgaagacatggcctgcaagtatgcgaatcccccggtcagcgtgaaggctgacttcggtgccgtcatggatgtcctcaagaagaggctcaagggtaagctctga

SEQ ID NO: 124 - protein, 521 aa, Oryza sativa
MAATTHAASLSFLLSHPHPTSPNPNPNPNLPLRRAPHRVRCATDAAATRHRRAADENIREEAARHRAPNHNFSAWYAPFPPAPNGDPDERYSLDEIVYRSSSGGLLDVRHDMDALARFPGSYWRDLFDSRVGRTTWPFGSGVWSKKEFVLPEIDPDHIVSLFEGNSNLFWAERLGRDHLAGMNDLWVKHCGISHTGSFKDLGMTVLVSQVNRLRRAPLSRPIAGVGCASTGDTSAALSAYCAAAGIPAIVFLPANRISLEQLIQPIANGATVLSLDTDFDGCMRLIREVTAELPIYLANSLNSLRLEGQKTAAIEILQQFDWEVPDWVIVPGGNLGNIYAFYKGFEMCRVLGLVDRVPRLVCAQAANANPLYRYYKSGWTEFTPQVAEPTFASAIQIGDPVSVDRAVVALKATDGIVEEATEEELMNAMSLADRTGMFACPHTGVALAALFKLRDQRIIGPNDRTVVVSTAHGLKFSQSKIDYHDSKIEDMACKYANPPVSVKADFGAVMDVLKKRLKGKL

SEQ ID NO: 125 - DNA, 1584 bp, Zea mays
atggcgaccttcaccgcggcctcctccctctccctcctcttctcccacccgcactcccactcccgccaaccatccgcccaggggcccaccgccagctcccacctccacctgcatccgcgcgccagccgcgcgcgctgcgcctcttccgacacgacggccacgaagcaccgccgcccagcggaggagaacatccgcgaggaggcggcgcggctccgaggcccggcccagggtttctctgcgtggtacgagcccttcccgccggcgcccggcggcgacccgaacgagcgctactcgctggacgaggtcgtctaccgctccagctcggggggcctcctcgacgtgcgccacgacatggaggcgctggcccgctacccggggtcctactggcgtgacctcttcgactcccgcgtcggccgcaccgcctggccctacggctcgggcgtctggtccaagaaggagttcgtgctccccgagatcgactccgaccacatcgtctccctcttcgagggcaactccaacctcttctgggcggagcgcctcggccgcgagcacctcggcgggatgaacgacctctgggtcaagcactgtggcatctcccacacgggctccttcaaggacctcggcatgacggtgctcgtcagccaggtgaaccgcctccgccgcgcgccgctctcgcgccccatcgccggtgtcggctgcgcgtccacgggagacacctccgccgcgctctcggcctactgcgcagccgcgggaatccccgccatcgtgttcctgccagcggaccgcatctcgctgcagcagctcatccagccgatcgccaacggcgccaccgtgctctctctagacactgattttgatggctgcatgcggctcattcgcgaggtcactgcagagctgccaatctaccttgccaattcgctcaacccgctccgccttgaggggcagaagacagcggccatcgagatattgcagcagttcaattggcaggtgccagattgggtcattgttccaggaggcaatcttgggaatatctatgcattctacaaggggtttgagatgtgccgcgttcttggacttgttgatcgcgtgccacggcttgtctgcgcacaggctgcaaatgcaaatccattgtaccggtactacaagtcaggttggactgagtttgagccacaaactgccgagactacatttgcatctgcgatacagattggtgatcctgtatctgttgaccgtgcggtggtcgcgctgaaggccactgacggtattgtggaggaggctacagaggaggagctaatggatgcaacggcgcttgctgaccgcactgggatgtttgcttgcccacatactggggttgcacttgctgctttgtttaagcttcagggtcagcgtataattggccctaatgaccgcactgtggttgttagcacagctcatgggctgaagttcacgcagtcaaagattgactaccatgacaaaaacatcaaagacatggtttgccagtatgctaatccaccgatcagtgtgaaggctgactttggttctgtgatggatgttctccagaaaaatctcaatggtaagatataa

SEQ ID NO: 126 - protein, 527 aa, Zea mays
MATFTAASSLSLLFSHPHSHSRQPSAQGPTASSHLHLHPRASRARCASSDTTATKHRRPAEENIREEAARLRGPAQGFSAWYEPFPPAPGGDPNERYSLDEVVYRSSSGGLLDVRHDMEALARYPGSYWRDLFDSRVGRTAWPYGSGVWSKKEFVLPEIDSDHIVSLFEGNSNLFWAERLGREHLGGMNDLWVKHCGISHTGSFKDLGMTVLVSQVNRLRRAPLSRPIAGVGCASTGDTSAALSAYCAAAGIPAIVFLPADRISLQQLIQPIANGATVLSLDTDFDGCMRLIREVTAELPIYLANSLNPLRLEGQKTAAIEILQQFNWQVPDWVIVPGGNLGNIYAFYKGFEMCRVLGLVDRVPRLVCAQAANANPLYRYYKSGWTEFEPQTAETTFASAIQIGDPVSVDRAVVALKATDGIVEEATEEELMDATALADRTGMFACPHTGVALAALFKLQGQRIIGPNDRTVVVSTAHGLKFTQSKIDYHDKNIKDMVCQYANPPISVKADFGSVMDVLQKNLNGKI

SEQ ID NO: 127 - DNA, 1593 bp, Sorghum bicolor
atggcgaccttcaccgcggcctcctccctctccctcctcttctcccaccccaactcccactcccgccaaccatccgtgcgcggggggcccgccgccggctcccacctccgcctgcctccccgcgccagccccagccgcgcgcgctgcgcctcctccgacacgacggccacgaagcaccgccgcccagcggaggagaacatccgcgaggaggcggcgcggctccggggccccgcgcagggcttctcggcgtggtacgagcccttcccgccggcgcccggcggcgaccccgacgagcgctactcgctggacgaggtcgtctaccgctccagctcggggggcctcctcgacgtgcgccacgacatggaggcgctggcgcgctacccgggctcctactggcgcgacctcttcgactcccgcgtcggccgcaccgcctggccctacggctcgggcgtctggtccaagaaggagttcgtgctccccgagatcgactccgaccacatcgtctccctcttcgagggcaactccaacctcttctgggcggagcgcctcggccgcgagcacctcggcgggatgaacgacctctgggtcaagcactgcggcatctcccacacgggctccttcaaggacctcggcatgaccgtgctcgtcagtcaggtgaaccgcctccgccgcgcgccgctctcgcgccccatcaacggtgtcggctgtgcgtccacgggagacacctccgccgcgctctcggcctactgcgcggccgcgggaatccccgccatcgtgttcctgccagcggaccgcatctcgctgcagcagctcatccagccaatcgccaacggcgccaccgtgctctctctagacactgattttgatggctgcatgcgactcattcgcgaggtgactgcagagctgccaatctaccttgccaattcactcaactcgcttcgcctcgaggggcagaagacagcggccatcgagatattgcagcagttcaattggcaggtgccggattgggtcattgttccaggaggcaatcttgggaatatctatgcattctacaaggggtttgagatgtgccgcgttcttggccttgttgatcgtgtgccacggcttgtctgtgcacaggctgcaaatgcaaatccgttgtaccggtactacaagtcaggctggactgagtttcagccacaagttgctgaaactacatatgcatctgcaatacagattggtgatcctgtatctgttgaccgtgcggtggtcgcgctgaaggctaccaatggtattgtggaggaggctacagaggaggagctaatggatgcgacggctcttgctgaccgcactgggatgtttgcttgcccacatactggggttgcacttgctgctttgtttaagctccgggatcagcgtataattgggcctaatgaccgcactgtggttgttagcacagctcatgggctgaagttcacgcagtcaaagatcgactaccatgacaaaaacatcaaggacatggtttgccagtatgctaatccaccgatcagtgtgaaggctgactttggttctgtgatggatgttctccagaaaaatctcaatggtaagatataa

SEQ ID NO: 128 - protein, 530 aa, Sorghum bicolor
MATFTAASSLSLLFSHPNSHSRQPSVRGGPAAGSHLRLPPRASPSRARCASSDTTATKHRRPAEENIREEAARLRGPAQGFSAWYEPFPPAPGGDPDERYSLDEVVYRSSSGGLLDVRHDMEALARYPGSYWRDLFDSRVGRTAWPYGSGVWSKKEFVLPEIDSDHIVSLFEGNSNLFWAERLGREHLGGMNDLWVKHCGISHTGSFKDLGMTVLVSQVNRLRRAPLSRPINGVGCASTGDTSAALSAYCAAAGIPAIVFLPADRISLQQLIQPIANGATVLSLDTDFDGCMRLIREVTAELPIYLANSLNSLRLEGQKTAAIEILQQFNWQVPDWVIVPGGNLGNIYAFYKGFEMCRVLGLVDRVPRLVCAQAANANPLYRYYKSGWTEFQPQVAETTYASAIQIGDPVSVDRAVVALKATNGIVEEATEEELMDATALADRTGMFACPHTGVALAALFKLRDQRIIGPNDRTVVVSTAHGLKFTQSKIDYHDKNIKDMVCQYANPPISVKADFGSVMDVLQKNLNGKI

SEQ ID NO: 129 - DNA, 1581 bp, Arabidopsis thaliana
atggcttcgtcttgtctcttcaatgcctctgtatcgtccttaaaccctaaacaagaccccatccgccgccaccggtcaacctctctcctccgccaccgccccgtcgtcatctcctgtaccgccgatggcaacaacatcaaagccccgatcgagacagcggtcaagcctcctcaccgtaccgaggataacattcgagatgaggctcgtcgtaatcgttccaacgccgtgaatccattttcagctaaatacgttccgtttaatgcagctcctggatccacggagtcttactctctcgacgagatcgtgtaccgtagccgctccggtggtttgcttgatgtcgaacacgatatggaggctttgaagcgattcgatggcgcgtattggcgtgatctcttcgattcgcgtgttggtaaaagcacatggccttatggatcgggtgtttggtcgaagaaagagtgggttcttcctgagatcgatgacgacgacatcgtttcagcttttgaaggaaactcgaatctgttctgggcagagagatttggtaagcagtttctaggtatgaatgatctgtgggtgaaacactgtgggattagtcatacaggaagtttcaaggatcttggaatgactgttttggttagtcaagttaatcgtctgagaaagatgaaacgacctgtggttggtgtcggatgtgcttccaccggagatacttctgctgctctatctgcttactgcgcctccgctggaatcccatcgattgtgtttttaccggcgaacaagatctctatggctcagctggttcagccgatagctaatggtgcgtttgttttgagtattgacactgattttgatgggtgtatgaagctgattagagagataactgcggaattgccgatttatttggcgaattcgttgaatagtttgaggttagaagggcagaaaactgcagctattgagattttgcagcagtttgattggcaagttcctgattgggtgattgttcctggaggtaacctaggaaacatctatgccttttacaaagggtttaagatgtgtcaagaactgggacttgtcgataggatcccgaggatggtctgtgcacaagcagctaatgctaatcctctttacttgcactacaagtctggttggaaggacttcaagcccatgactgcaagtaccactttcgcctctgcgattcagatcggtgaccctgtctccatcgatagagctgtgtacgctctcaagaagtgcaatggtattgtagaagaagccacagaggaggagctgatggatgcgatggctcaagcggattcgacaggaatgtttatctgtcctcatacaggtgttgctctaactgctctgttcaagctgaggaatcaaggagtgattgcaccgactgatcgaactgtggtagtgagtactgctcatgggttgaagtttactcagtctaagatagattatcactccaatgccatccctgacatggcttgcagattctccaatcctcctgttgatgtgaaagcagatttcggagctgtcatggatgttctcaagagttacttaggaagtaatacacttacgtcataa

SEQ ID NO: 130 - protein, 526 aa, Arabidopsis thaliana
MASSCLFNASVSSLNPKQDPIRRHRSTSLLRHRPVVISCTADGNNIKAPIETAVKPPHRTEDNIRDEARRNRSNAVNPFSAKYVPFNAAPGSTESYSLDEIVYRSRSGGLLDVEHDMEALKRFDGAYWRDLFDSRVGKSTWPYGSGVWSKKEWVLPEIDDDDIVSAFEGNSNLFWAERFGKQFLGMNDLWVKHCGISHTGSFKDLGMTVLVSQVNRLRKMKRPVVGVGCASTGDTSAALSAYCASAGIPSIVFLPANKISMAQLVQPIANGAFVLSIDTDFDGCMKLIREITAELPIYLANSLNSLRLEGQKTAAIEILQQFDWQVPDWVIVPGGNLGNIYAFYKGFKMCQELGLVDRIPRMVCAQAANANPLYLHYKSGWKDFKPMTASTTFASAIQIGDPVSIDRAVYALKKCNGIVEEATEEELMDAMAQADSTGMFICPHTGVALTALFKLRNQGVIAPTDRTVVVSTAHGLKFTQSKIDYHSNAIPDMACRFSNPPVDVKADFGAVMDVLKSYLGSNTLTS

SEQ ID NO: 131 - DNA, 1560 bp, Glycine max
atggcttcctcttctctgtttcagtctctccctttctctctccaaacctctaaaccctacgcgcctcccaaacccgccgcccacttcgttgtccgcgcccaatcccccctcactcagaacaacaactcctcctccaagcatcgccgccccgccgacgagaacatccgcgacgaggcccgccgcatcaatgcgccccacgaccaccacctcttctcggccaagtacgtccccttcaacgccgactcctcctcctcctcctccacggagtcctactcgctcgacgagatcgtctaccgctcccaatccggcggcctcctggacgtccagcacgacatggatgccctcaagcgtttcgacggcgagtactggcgcaacctcttcgactcgcgcgtgggcaaaaccacctggccttacggctccggcgtctggagcaaaaaagaatgggtcctccccgagatccacgacgacgatatcgtctccgccttcgagggtaactccaacctcttctgggccgagcgtttcggcaaacagttcctcggcatgaacgatttgtgggtcaaacactgcggaatcagccacaccggcagcttcaaggatctcggcatgaccgtcctcgtcagccaggtcaatcgcttgagaaaaatgaaccgccccgtcgtcggtgttggttgcgcctccaccggtgacacatcggccgctttatccgcctattgcgcttccgctgccattccttccattgtgtttttgcctgctaataaaatctctcttgcccaacttgttcagcctattgccaatggagcctttgtgttgagtatcgacactgattttgatggttgcatgcagttgatcagagaggtcactgctgagttgcctatttatttggctaactctctcaacagtttgaggttggaagggcagaagactgctgctattgagattctgcagcagtttgattggcaggttcctgattgggtcattgtgcctggaggcaaccttggcaacatttatgccttttacaaagggtttaagatgtgtcaagagcttgggcttgtggataagattccaaggcttgtttgtgctcaggctgccaatgctgatcctttgtatttgtactttaaatccgggtggaaggagtttaagcctgtgaagtcgagcactacatttgcctctgccattcaaattggtgatcctgtttccattgacagggcggttcacgcgctaaagagttgcgatgggattgtggaggaggccacggaggaggagttgatggatgctacagcgcaggcggattctactgggatgtttatttgcccccacaccggggttgctttaactgcattgtttaagctcaggaacagcggggttattaaggccactgataggactgtggtggttagcactgctcatggcttgaagttcactcagtccaagattgattaccattctaaggacatcaaggacatggcttgccgctatgctaacccgcccatgcaagtgaaggcagactttggctcggttatggatgttttgaagacgtatttgcagagtaaggctcattag

SEQ ID NO: 132 - protein, 519 aa, Glycine max
MASSSLFQSLPFSLQTSKPYAPPKPAAHFVVRAQSPLTQNNNSSSKHRRPADENIRDEARRINAPHDHHLFSAKYVPFNADSSSSSSTESYSLDEIVYRSQSGGLLDVQHDMDALKRFDGEYWRNLFDSRVGKTTWPYGSGVWSKKEWVLPEIHDDDIVSAFEGNSNLFWAERFGKQFLGMNDLWVKHCGISHTGSFKDLGMTVLVSQVNRLRKMNRPVVGVGCASTGDTSAALSAYCASAAIPSIVFLPANKISLAQLVQPIANGAFVLSIDTDFDGCMQLIREVTAELPIYLANSLNSLRLEGQKTAAIEILQQFDWQVPDWVIVPGGNLGNIYAFYKGFKMCQELGLVDKIPRLVCAQAANADPLYLYFKSGWKEFKPVKSSTTFASAIQIGDPVSIDRAVHALKSCDGIVEEATEEELMDATAQADSTGMFICPHTGVALTALFKLRNSGVIKATDRTVVVSTAHGLKFTQSKIDYHSKDIKDMACRYANPPMQVKADFGSVMDVLKTYLQSKAH

SEQ ID NO: 133 - DNA, 393 bp, Oryza sativa
atgggagagcaaggaggcagggcaagcagcaacaagatcagggacattgtgaggctgcaccagcttctcaagaggtggaagagggctgcacttgcaccaaaggccggcaagaacaacaatggcggcggtgcatcggtcccgaaagggttcttcgcggtgtgcgtcggggaggagatgaggaggtttgtcatccccacagagtatcttggccactgggcatttgagcagctactcaggaaggcagaggaggagtttgggttccagcatgagggagctctgaggattccatgtgatgttgaggtgtttgagggtatcttgaggctggttggcaggaaggatgagaaggcagcaatgtgctactcttcttcagagcatgagatcttgtgcagatga

SEQ ID NO: 134 - protein, 130 aa, Oryza sativa
MGEQGGRASSNKIRDIVRLHQLLKRWKRAALAPKAGKNNNGGGASVPKGFFAVCVGEEMRRFVIPTEYLGHWAFEQLLRKAEEEFGFQHEGALRIPCDVEVFEGILRLVGRKDEKAAMCYSSSEHEILCR

SEQ ID NO: 135 - DNA, 399 bp, Zea mays
atgggggagcaaggcaggccaagcagcaacaggatcagagacatcgtgaggctgcgacagcttctcaagaagtggaagcagattgcgctctcaccgaaagccggcaagagcggcggcggcggcggcagccacggtgtcccgaaggggttcttcacggtgtgcgtcggcaaggagatggagaggttcgtgatccccacggagtacctgggccactgggcgttcgaggagctcctgaaggaggcggaggaggagttcgggttccagcacgagggggctctcaggatcccctgcgacgtgaaggcgttcgagggcatcctgaggctggtgggcaggaaggatgcggcggctgcggatcgctactgttcttcgcagcatgggatgatgatcttgtgcagatga

SEQ ID NO: 136 - protein, 132 aa, Zea mays
MGEQGRPSSNRIRDIVRLRQLLKKWKQIALSPKAGKSGGGGGSHGVPKGFFTVCVGKEMERFVIPTEYLGHWAFEELLKEAEEEFGFQHEGALRIPCDVKAFEGILRLVGRKDAAAADRYCSSQHGMMILCR

SEQ ID NO: 137 - DNA, 375 bp, Sorghum bicolor
atgggggagcaaggcaggtccagcagcaacaagatcagagacattgtgaggctgcaacaacttctgaagaagtggaagcggcttgcactctcgccaaaagccggcaagagcagcagcaaccatggtgttccaaagggtttctttgcggtgtgcgttggcatggagatgaagaggtttgtgatccccacggagtacctaggccactgggcatttgaggagctcctgaaggaggcagaggaggaatttggattccagcatgagggagctctgagaatcccctgtgatgtgaaggtgtttgagggcatcctcaggctggtgggcaggaaggaggcagtttgctacagtccttcacagcctgggatcttatgcagataa

SEQ ID NO: 138 - protein, 124 aa, Sorghum bicolor
MGEQGRSSSNKIRDIVRLQQLLKKWKRLALSPKAGKSSSNHGVPKGFFAVCVGMEMKRFVIPTEYLGHWAFEELLKEAEEEFGFQHEGALRIPCDVKVFEGILRLVGRKEAVCYSPSQPGILCR

SEQ ID NO: 139 - DNA, 570 bp, Arabidopsis thaliana
atggaggccaagaagtcaaacaaaatcagagagatcgttaagcttcaacagatcctcaagaaatggcgaaaagttgcacacgcatcaaaacaagccaacaacaacaagatcgacaacgtagatgacagcaacaacaacatcagcatcaacatcaacaacaatggaagtggaagtggaagtggaagcaagagcatcaagtttctgaagagaacactatccttcacagacacaacagctattcctaaaggctacttagctgtctcggtggggaaggaggagaaaagatacaagataccaacagagtaccttagccaccaagctttccatgtgctgttgcgtgaagcagaagaagagtttgggtttgaacaagctggtatcttgaggattccttgtgaagttgctgtgttcgagagcattttgaagataatggaggacaacaagagtgatgcgtacctgaccactcaagagtgcagattcaatgccacaagtgaggaagtgatgagttatcgtcatccttcggattgcccgaggacaccatctcaccaacctcacagcccaatgtgcagatag

SEQ ID NO: 140 - protein, 189 aa, Arabidopsis thaliana
MEAKKSNKIREIVKLQQILKKWRKVAHASKQANNNKIDNVDDSNNNISININNNGSGSGSGSKSIKFLKRTLSFTDTTAIPKGYLAVSVGKEEKRYKIPTEYLSHQAFHVLLREAEEEFGFEQAGILRIPCEVAVFESILKIMEDNKSDAYLTTQECRFNATSEEVMSYRHPSDCPRTPSHQPHSPMCR

SEQ ID NO: 141 - DNA, 534 bp, Glycine max
atgtcttctatggatctaaagaaatctaacaagatcagagaaattgttaggcttcaacagatcctcaagaaatggagaaagttagccaactcatcaaaaaccactatggttaccaccaccgctaccgccactgtcacctcttccgccagcaagagcatgaagtatcttaagagaacactttccctatcagaacgtgaaggagggtcaagcaatgtagtccccaaagggtacctagctgtttgtgttggtgaagagctcaagaggttcactataccaactgaatatttaggtcatcaagcctttcagattctcctcagagaagcagaagaagaatttggctttcaacaaaccggagttctgaggattccttgtgaagtggctgtttttgagagcatcttgaagatggtggaaggaaaggaggacaagttttcctcccaagaatgtagactcagcattgaagaaatgatgatgggttaccgctccgaaaaccaacttgcttattctcaccatcctcaaagtccactgtgcagatag

SEQ ID NO: 142 - protein, 177 aa, Glycine max
MSSMDLKKSNKIREIVRLQQILKKWRKLANSSKTTMVTTTATATVTSSASKSMKYLKRTLSLSEREGGSSNVVPKGYLAVCVGEELKRFTIPTEYLGHQAFQILLREAEEEFGFQQTGVLRIPCEVAVFESILKMVEGKEDKFSSQECRLSIEEMMMGYRSENQLAYSHHPQSPLCR

SEQ ID NO: 143 - DNA, 882 bp, Oryza sativa
atggccgaccgcgtctacccggccgcgaagcccaacccaccgccggcaatggcgaacgcgggcggcggcggcgcgacggcgtcgttcccggcgcccaagtcgcagatgtaccagcggccaatctaccggccgcaggcggcggcggcgaagcggcggcgcgggcgttcctgccgatgcagcttctgctgctgcttctgctgggcgctgctggtcgtcatcctcctggcgctcgtcgccgccgtcgccggcggcgcgttctacctgctctaccgcccgcaccgccccagcttcaccgtctcgtccgtcaagctcaccgcgctcaacctctcgtcgtcgcccacctcgccgtcgctcaccgactccatccagctcaccgtcaccgccaagaaccccaacaagaaggtcgtctacctctacgacgacttctccttctccgcctccaccgccgccaacgccgtcccgctcggcgccgccacgtcgccgggcttcacccacgacgccggcaacaccaccgtcttcaccgccaccatcgccgccaacgccgtcgccgtcgacccggccgccgccgcctccgacatcaagaagtccggcgccttctccgtcgccgtcgacgccgagacgcgcgccggcgtcagggtgggcagcctcaagaccaagaagatcggcatccaggtgcactgcgagggcatcaaggtgacgccgccgccgcccgccgccctgccgcgccccaaggcggtgaaggggaagaacggcaccgtgctggctccggcgccggcgccggcggactccgacacggcggcgaccaccgccgcgacggtgagcaccgcggcgcactcgtgcaaggtcagagtccgtgtcaagatctggaagtggaccttttag

SEQ ID NO: 144 - protein, 293 aa, Oryza sativa
MADRVYPAAKPNPPPAMANAGGGGATASFPAPKSQMYQRPIYRPQAAAAKRRRGRSCRCSFCCCFCWALLVVILLALVAAVAGGAFYLLYRPHRPSFTVSSVKLTALNLSSSPTSPSLTDSIQLTVTAKNPNKKVVYLYDDFSFSASTAANAVPLGAATSPGFTHDAGNTTVFTATIAANAVAVDPAAAASDIKKSGAFSVAVDAETRAGVRVGSLKTKKIGIQVHCEGIKVTPPPPAALPRPKAVKGKNGTVLAPAPAPADSDTAATTAATVSTAAHSCKVRVRVKIWKWTF

SEQ ID NO: 145 - DNA, 936 bp, Zea mays
atgggcgaccgggcgtacgcgccggccgtgaagccggttcccgtgcgggccaccaacggcaccgcgaacggcggcggcgtggggcctccgcggcccgcgccgccgtccatggtgcccggcgggcgcgtgccccctccgccgatgtacaggcggaggcccgcgcagtcgcgtcctccggcgcggcgtgccgggcggagcgcccgcgggtggtgctgcgcgtgctgcctgtggctgacgctggtgctggtggggctggcgttcctgggcgccatcgcggcgggggtgttctacgtggtgtaccggccgcggccgcccagcttcgcggtgacgtcggtgcggctggcggcgctgaacgtgtcggactcggacgcgctcacctcccgcgtggagttcacggtgacggcgcggaacccgaacgacaagatcgccttcgactacggcgacatggcggtgtccttcgcctcgggcggcgcggacgtgggcgacgccgtggtcccggggttcctccacccggcgggcaacacgacggtcatccgcgccgccgcgtccaccgccgcgtccaccatcgaccccgtccaggcggcggcgctcagatccaggaagtcccacgtgatgtcggcgcagatggacgccaaggtcgggttccagatcgggcggtccaagtccaagagcatcaacgtccgcgtcagctgcgcgggggtctccgttgggctcgccaagccggctccggctccggctgcggccgcgcccgcgcccgcgcccgcgccggacgcggagccggccccggcccgcggccgtgggcgtgggcggtcgccgcggtcggtcgtacggacgtcctcctcctcctcctcctccggcggcggtggcggcgggaagttgacgccgacggacgcaaagtgtaaggtccgcatcaagatctggatttggtcgttttga

SEQ ID NO: 146 - protein, 311 aa, Zea mays
MGDRAYAPAVKPVPVRATNGTANGGGVGPPRPAPPSMVPGGRVPPPPMYRRRPAQSRPPARRAGRSARGWCCACCLWLTLVLVGLAFLGAIAAGVFYVVYRPRPPSFAVTSVRLAALNVSDSDALTSRVEFTVTARNPNDKIAFDYGDMAVSFASGGADVGDAVVPGFLHPAGNTTVIRAAASTAASTIDPVQAAALRSRKSHVMSAQMDAKVGFQIGRSKSKSINVRVSCAGVSVGLAKPAPAPAAAAPAPAPAPDAEPAPARGRGRGRSPRSVVRTSSSSSSSGGGGGGKLTPTDAKCKVRIKIWIWSF

SEQ ID NO: 147 - DNA, 939 bp, Sorghum bicolor
atgggcgaccgggcgtacgcgccggccgcgaagccggttcccgtgcgcgccaccaacggcaccgcgaacggcggcggcggcggtcccccgcgtcccgcgccgccgtccatgctgcccggcggtcgcgtgccccctccgccgatgtaccgtccgaagcccgcgcagtcgcgccctccggcgcgccgcccccgccggagcgcccgcgggtggtgctgcgcgtgctgcctgtggctgacgctggtgctggtgggcctggtgttcctgggcgccatcgcggcgggggtgttctacgtggtgtaccgcccgcgcccgcccagcttcgcggtgacgtcgctgcgcctggcggcgctgaacgtgtcggactcggacgcgctcacctcccgcatcgagttcacggtgacggcgcggaaccccaacgacaagatcgccttccgctacggcgacatcgcggcgtccttcgcctccgacgacggcgccgacgtgggcgacggcgtggtcccgggcttcctccacccggcgggcaacaccaccgtcgtccgcgccgcggcctccaccgcgtcgtccaccatcgaccccgtccaggcggcggcgctcagatccagaaagtcccacgtcatggccgcgcagatggacgccaaggtcggcttccagatcgggcggttcaagtccaagagcatcaacgtgcgcgtcacctgcgcgggggtctccgtggggctcgccaagccgcctcccgccgccgcgcccgcgcccgcgccggacgcggagccgaccgtcgtggtcgccgcggcgccggcgcccgcccgaggccgtgggcgtgggcggtcgccgcggtcggtcgtacggacgtcgtcctccagcgccagcggcggcggagggaagatgacgccgacggacgcaaagtgtaaggtccgcatcaagatctggatttggtcgttttga

SEQ ID NO: 148 - protein, 312 aa, Sorghum bicolor
MGDRAYAPAAKPVPVRATNGTANGGGGGPPRPAPPSMLPGGRVPPPPMYRPKPAQSRPPARRPRRSARGWCCACCLWLTLVLVGLVFLGAIAAGVFYVVYRPRPPSFAVTSLRLAALNVSDSDALTSRIEFTVTARNPNDKIAFRYGDIAASFASDDGADVGDGVVPGFLHPAGNTTVVRAAASTASSTIDPVQAAALRSRKSHVMAAQMDAKVGFQIGRFKSKSINVRVTCAGVSVGLAKPPPAAAPAPAPDAEPTVVVAAAPAPARGRGRGRSPRSVVRTSSSSASGGGGKMTPTDAKCKVRIKIWIWSF

SEQ ID NO: 149 - DNA, 795 bp, Arabidopsis thaliana
atgacagacgacagagtttaccctgcatcaaaacctcccgccatcgtcggtggcggtgccccaaccaccaatccaactttcccggcgaacaaagctcagctctacaacgcaaatcgtcccgcttaccgtccaccagctggtcgtcgtcgtactagccatacccgtggatgttgctgccgttgctgttgctggacgatattcgtaatcatcctcttactcctcatcgtcgccgccgcatcagccgtcgtatacctaatctaccgtcctcaacgacctagcttcaccgtctctgaactcaaaatctccactctcaacttcacatccgccgttcgcctcaccaccgccatttccctctccgtcatcgccagaaaccctaacaaaaacgttggattcatctacgacgtcaccgacatcacactctacaaagcatccaccggaggagatgatgacgtagtcattggtaaaggaacgatcgcggcgttttctcacgggaagaagaacacgactacgcttagaagtacgatcggaagtcctccggatgaactcgatgagatctcggcgggtaagctgaaaggagatctgaaggcgaagaaagcagtggcgattaagattgttttgaactcgaaggtgaaagtgaagatgggagctctaaaaactcctaaatcaggaattagggttacttgtgaagggattaaagtggtggctccgacgggaaagaaggcgacgacggctacgacttccgccgctaagtgtaaggttgatccaagatttaagatctggaaaattactttctaa

SEQ ID NO: 150 - protein, 264 aa, Arabidopsis thaliana
MTDDRVYPASKPPAIVGGGAPTTNPTFPANKAQLYNANRPAYRPPAGRRRTSHTRGCCCRCCCWTIFVIILLLLIVAAASAVVYLIYRPQRPSFTVSELKISTLNFTSAVRLTTAISLSVIARNPNKNVGFIYDVTDITLYKASTGGDDDVVIGKGTIAAFSHGKKNTTTLRSTIGSPPDELDEISAGKLKGDLKAKKAVAIKIVLNSKVKVKMGALKTPKSGIRVTCEGIKVVAPTGKKATTATTSAAKCKVDPRFKIWKITF

SEQ ID NO: 151 - DNA, 765 bp, Glycine max
atgactgatagggttcacccttcggccaaaaccaccgccaacgccggccccaagccgacattccccgctacgaaatcccagctttccggcgccaaccgccccacctaccgcccccaaccgcagcaccaccgccgccgccgtagtcgcggatgtgcctccaccctctgctgctggctcctcctgatcctcctcttcctcctcctcctcgtcggtgccgccggcaccgtcctctactttctctaccgtccccaacgacccacattctccgtcacctccctaaaactctcttccttcaacctcaccactccctccaccatcaacgccaagtttgacctcactctctcaacaactaaccctaacgacaaaatcatcttctcctacgaccctacctccgtatcccttctctacggcgacaccgccgtcgccagcaccaccatcccctccttcctccaccgccaaaggaacaccaccgtgctccaggcttatgttactagcactgaggaagtggtggatagtgacgccgcgatggagctgaagaggagcatgaagaggaagagtcagctggtggcgctgaaggtggagctggagaccaaggtggaggcccagatgggcgtgttccagacgcctcgagtcgggatcaaggttctgtgcgacggcgtcgccgtatctctccccgacgatgagaaaccggcgacggcgtcggctgagaatacggcgtgccaggtggatgtgaggtttaaggtctggaaatggaccgttggatga

SEQ ID NO: 152 - protein, 254 aa, Glycine max
MTDRVHPSAKTTANAGPKPTFPATKSQLSGANRPTYRPQPQHHRRRRSRGCASTLCCWLLLILLFLLLLVGAAGTVLYFLYRPQRPTFSVTSLKLSSFNLTTPSTINAKFDLTLSTTNPNDKIIFSYDPTSVSLLYGDTAVASTTIPSFLHRQRNTTVLQAYVTSTEEVVDSDAAMELKRSMKRKSQLVALKVELETKVEAQMGVFQTPRVGIKVLCDGVAVSLPDDEKPATASAENTACQVDVRFKVWKWTVG